(19) United States
(12) Patent Application Publication
(10) Pub. No.: US 2022/0187282 A1
(43) Pub. Date: Jun. 16, 2022

(54) CARDIOMYOCYTE PROLIFERATION

(71) Applicant: The Council of the Queensland Institute of Medical Research, Herston (AU)

(72) Inventors: James HUDSON, Herston (AU); Richard MILLS, Herston (AU); Enzo PORRELLO, Brisbane (AU); Gregory QUAIFE-RYAN, Herston (AU)

(21) Appl. No.: 17/440,081

(22) PCT Filed: Mar. 18, 2019
(86) PCT No.: PCT/AU2019/050238

§ 371 Date: Sep. 16, 2021

(51) Int. Cl. G01N 33/50; C12N 5/077

(52) U.S. Cl. CPC G01N 33/5061; C12N 5/0657

(57) ABSTRACT

Provided herein are in vitro and in vivo methods of inducing cardiomyocyte proliferation by contacting cardiomyocytes with or administering to a subject an effective amount of an agent capable of activating sterol biosynthesis, such as mevalonate biosynthesis. Methods and compositions for regenerating a cardiac tissue in a subject that include administering thereto a therapeutically effective amount of an agent capable of activating sterol biosynthesis in a cardiomyocyte are also provided herein.

### TECHNICAL FIELD

THIS INVENTION relates to cardiomyocytes. More particularly, this invention relates to a method and composition that promotes cardiomyocyte proliferation, which may be used for treating or repairing cardiac damage in a subject in need thereof.

### BACKGROUND

Induction of adult cardiomyocyte proliferation has emerged as a key strategy to promote endogenous heart regeneration following cardiac injury. Recent studies have provided evidence for near complete restoration of cardiac function following stimulation of adult cardiomyocyte proliferation in mouse models of ischemic heart disease and heart failure (Bassat et al., 2017; D'Uva et al., 2015; Leach et al., 2017; Mohamed et al., 2018; Nakada et al., 2017). These studies have stimulated considerable interest in the development of therapeutic modalities to enhance cardiomyocyte proliferation and promote endogenous regeneration of the human heart following damage. However, translation of these important biological discoveries into the clinic will likely require drug development, validation of mouse findings in a relevant human model system and avoidance of potential side-effects of pro-proliferative agents on cardiac contractility and rhythm.

### SUMMARY

The invention is broadly directed to a method or composition that promotes or stimulates cardiomyocyte proliferation in vitro or in vivo. The invention is also broadly directed to a method of treating or repairing cardiac damage in a subject by stimulating said cardiomyocyte proliferation therein.

A first aspect of the invention provides a method of inducing cardiomyocyte proliferation in vitro, the method including the step of contacting one or a plurality of cardiomyocytes with an effective amount of an agent capable of at least partly activating sterol biosynthesis therein to thereby induce cardiomyocyte proliferation.

A second aspect of the invention provides a method of inducing cardiomyocyte proliferation in a subject, the method including the step of administering to the subject an effective amount of an agent capable of at least partly activating sterol biosynthesis in a cardiomyocyte to thereby induce cardiomyocyte proliferation in the subject.

A third aspect of the invention provides a method of regenerating a cardiac tissue in a subject in need thereof, the method including the step of administering to the subject a therapeutically effective amount of an agent capable of at least partly activating sterol biosynthesis in a cardiomyocyte to thereby treat or repair the cardiac damage in the subject.

Suitably, the agent is capable of promoting or inducing cardiomyocyte proliferation in the subject.

In certain embodiments, the subject has or is at risk of developing a cardiac disease, disorder or condition selected from the group consisting of a myocardial infarction, a congestive heart failure, tachyarrhythmia, familial hypertrophic cardiomyopathy, ischemic heart disease, idiopathic dilated cardiomyopathy, congenital heart disease and myocarditis.

With respect to the second and third aspects, administering the agent suitably comprises oral administration, intravenous injection, topical administration, myocardial injection, an implantable device and any combination thereof.

Referring to the aforementioned aspects, the agent suitably is or comprises a p38α inhibitor, a MST1 inhibitor, a TGF-beta receptor inhibitor and/or a BMP receptor inhibitor.

For the aforementioned aspects, activating sterol biosynthesis suitably comprises, at least in part, increasing the expression and/or activity of one or more proteins and/or enzymes of, or associated with, sterol biosynthesis. Preferably, the one or more proteins and/or enzymes are selected from the group consisting of squalene monooxygenase (SQLE), Hydroxymethylglutaryl(HMG)-CoA synthase (HMGCS1), Lanosterol 14 alpha-demethylase (CYP51A1), HMG-CoA reductase (HMGCR), Hydroxymethylglutaryl(HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase, a farnesyltransferase, Sterol regulatory element-binding protein 1 (SREBP1), Sterol regulatory element-binding protein 2 (SREBP2) and any combination thereof.

With respect to the above aspects, the agent is suitably further capable of at least partly modulating the expression and/or activity of a cell cycle protein. To this end, the cell cycle protein is preferably selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

In regard to the method of the aforementioned aspects, the agent suitably maintains, at least in part, contractile function of proliferated cardiomyocytes.

In a fourth aspect, the invention provides a composition for use in regenerating a cardiac tissue in a subject, the composition comprising a therapeutically effective amount of an agent capable of activating sterol biosynthesis and optionally a pharmaceutically acceptable carrier, diluent or excipient.

Suitably, the composition of the present aspect is for use in the method of first, second and third aspects.

In a fifth aspect, the invention provides a method of screening, designing, engineering or otherwise producing an agent for inducing cardiomyocyte proliferation, said method including steps of:

(a) contacting one or a plurality of cardiomyocytes with a candidate molecule; and

(b) determining whether the candidate molecule is capable of at least partly activating sterol biosynthesis to thereby induce cardiomyocyte proliferation.

Suitably, step (b) of the present method comprises determining whether the candidate molecule activates and/or increases the expression of one or more proteins and/or enzymes of, or associated with, sterol biosynthesis. Preferably, the one or more proteins and/or enzymes are selected from the group of squalene monooxygenase (SQLE), hydroxymethylglutaryl(HMG)-CoA synthase (HMGCS1), Lanosterol 14 alpha-demethylase (CYP51A1), HMG-CoA reductase (HMGCR), Hydroxymethylglutaryl (HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase, a farnesyltransferase, Sterol regulatory element-binding protein 1 (SREBP1), Sterol regulatory element-binding protein 2 (SREBP2) and any combination thereof.

In one embodiment, the present method includes the further step of determining whether the candidate molecule is capable of at least partly modulating the expression and/or activity of a cell cycle protein. In this regard, the cell cycle protein is suitably selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

Suitably, the one or plurality of cardiomyocytes are or comprise a cardiac organoid.

In a sixth aspect, the invention provides an agent for inducing cardiomyocyte proliferation screened, designed, engineered or otherwise produced according to the method of the fifth aspect.

Suitably, the agent of this aspect is for use according to the method of the first, second and third aspects.

Referring to the aforementioned aspects, activating sterol biosynthesis suitably comprises activating mevalonate biosynthesis and/or isoprenoid biosynthesis.

### BRIEF DESCRIPTION OF THE FIGURES

FIG. 1: Schematic outline of drug development strategy.
A 5,000 compound library was screened for pro-proliferative effects in iPSC-derived cardiomyocytes in 2D using EdU. The hits were eliminated if they also induced proliferation in fibroblasts.
Hit compounds were then screened in immature hCOs using Ki-67. The hits were eliminated if they decreased force or increased relaxation time.
Hit compounds were then screened in mature cell cycle arrested hCO using Ki-67.
Proliferation was confirmed by quantifying cardiomyocyte specific Ki-67 and pH3 immunostaining, counting cardiomyocyte number, analysing hCO size (without increased cardiomyocyte size), and excluding binucleation following treatment.

FIG. 2: Screening for proliferative activators that do not impact contractile force or relaxation time.

A. Screening protocol in immature hCO.
B. Ki-67 intensity in hCO treated with positive control CHIR99021 for 2 days. n=9 experiments.
C. Force of contraction in hCO treated with positive control CHIR99021 for 2 days. n=8 experiments.
D. Heat-map of hCO Ki-67 intensity after treatment with small molecules for 2 days. Molecules screened at 3 different concentrations with n=2-6 per concentration. E. Heat-map of hCO contractile function after treatment with small molecules for 2 days. Molecules screened at 3 different concentrations with n=2-6 per concentration.
F. Correlation of maximum effect on proliferation in 3D hCO versus 2D screening.
G. Correlation of maximum Ki-67 effect versus force of contraction in hCO.
H. Correlation of maximum Ki-67 effect versus 50% relaxation time (Tr) in hCO.
Triangles indicate hit compounds that reduce function, green triangles indicate compounds with GSK3 inhibition activity and purple triangles indicate compounds activating adenosine receptor 2A. Data is presented as mean±s.e.m. **, ****, denote P<0.01 and P<0.0001 respectively, using Mann-Whitney test B, C

FIG. 3: Follow-up of hits in secondary screen in mature hCO.
A. Schematic overview of protocol for hit validation.
B. Force of contraction following treatment of compounds for 2 days. n=6-10, 2 experiments.
C. Ki-67⁺ cardiomyocytes (α-actinin) following treatment with hits for 2 days. n=6-10, 2 experiments.
D. Mitosis (pH3) of cardiomyocytes (α-actinin) following 2 days of treatment of hits at optimal concentrations. n=17-19, 4 experiments for DMSO, compound 3 and compound 65 and n=6, 2 experiments compound 63.
E. Cardiomyocyte number in the mature hCOs following treatment with 3 μM compound 3 or 1 μM compound 65. n=11-13 from 2 experiments.
F. The size of the cardiomyocyte (α-actinin) area increases in hCO culture for 7 days with 3 μM compound 3 or 1 μM compound 65. n=12-16 from 3 experiments.
G. Structure of compound 3.
H. Structure of compound 65.
   Scale bars=20 μm. Data is presented as mean±s.e.m. *, **, ***, ****, denote P<0.05, P<0.01, P<0.001 and P<0.0001 respectively, using one-way ANOVA with Dunnett's post-test relative to DMSO in B, C, D, F or using two-way ANOVA E.

FIG. 4: Proliferation is activated by distinct processes for compound 3, 63 and 65.
A. Protocol. Mature hCO were stimulated with DMSO or compounds 3, 63 or 65 (at concentrations of 3, 1 or 0.3 μM, respectively) for 2 days.
B. Principal coordinate analysis of batch corrected RNA-sequencing data. n=10-20 hCO per condition from 3-4 experiments (number in brackets).
C. RNA-sequencing gene ontologies for compound 3.
D. RNA-sequencing gene ontologies compound 65.
E. Overlap of up-regulated proteins in RNA-sequencing and proteomics.
F. Key cell cycle genes changed in response to stimulation with the different compounds to activate proliferation leading to changes in the proteome. All hit compounds upregulated gene ontologies associated with G1/S transition and DNA replication, but only compounds 3 and 65 induced G2/M transition consistent with the induction of pH3 positive cardiomyocytes in FIG. 3D.
G. Each compound activated distinct “cell cycle” proteins in the proteome.
   Significantly regulated for RNA-seq analyses were FDR <0.10 or p LIMMA <0.05 for proteomics. For bubble plots size correlates to p-value (see legend in the top corner) and gene ontologies are represented in x and y in a principle component analysis showing relatedness.

FIG. 5: The core cell cycle program is correlated with activation of a cell cycle network and the mevalonate pathway.

A. Proteins upregulated in all proliferative conditions. *Note: aurora kinase B (AURKB) was also added to the cell cycle network as it was consistently up-regulated, but failed to reach significance for some treatments.
B. Expression of “proliferation signature” proteins (plus HMGCS1) in different conditions.
C. Cholesterol pathway proteins that increase with the strongest inducers, compounds 51 and 65.
D. Cholesterol genes HMGCR, HMGCS1, CYP51A1 (Cyp51 in the mouse) and SQLE expression during both mouse and human maturation in vivo. RNA-sequencing data extracted from data generated in previous studies (Kuppusamy et al., 2015; Mills et al., 2017a; Quaife-Ryan et al., 2017). Mouse data is for purified cardiomyocytes and all are significantly regulated (FDR <0.05). Human data for whole heart or hPSC-CM differentiation cultures and are significantly regulated from 20 day old hPSC-CM (adj p<0.05, using 2-way ANOVA with Tukey's post-test).
E. Cholesterol proteins Hmgcr, Hmgcs1, Cyp51 and Sqle showing a trend or statistically regulated (*) increases during heart regeneration in mice following delivery of constitutively active YAP1(S127A). Data from (Lin et al., 2014). MI—myocardial infarction.
F. Schematic showing that the cell cycle network is correlated with co-activation of the mevalonate pathway for full cell cycle progression.

FIG. 6: Mevalonate metabolic products are required for proliferation in hPSC-CM and mature hCO.
A. Experiments in 2D proliferative hPSC-CM (panels C-E).
B. Representative image from 2D experiments used for image cytometry and analysis.
C. Cardiomyocyte proliferation after 3 days of simvastatin treatment. n=12.
D. Cardiomyocyte number after 3 days of simvastatin treatment. n=12.
E. Cardiomyocyte size after 3 days of simvastatin treatment. n=12.
F. The mevalonate pathway. Red displays enzymes inhibited or metabolites added in G.
G. Cardiomyocyte proliferation after 24 hours. Mevalonate (Mev), geranyl-geranyl pyrophosphate (GGPP) and farnesyl pyrophosphate (FPP). n=12, 3 experiments.
H. Experiment in mature hCO (panels I,J).
I. Ki-67 intensity in mature hCO treated with 1 μM compound 65, 3 μM compound 3 or 10 μM compound 6.28 is abolished by 10 μM simvastatin. n=11-14, 2 experiments (compound 65) and n=8-22, 3 experiments (compound 3 and compound 6.28)
J. Simvastatin abolishes 3 μM compound 3 induced Ki-67⁺ cardiomyocytes in hCO. n=5-6.
   Scale bars=20 μm except for the full image in panel B which is 200 μm. Data is presented as mean±s.e.m. *, **, ***, ****, denote P<0.05, P<0.01, P<0.001, P<0.0001, respectively, using t-test for C-E,I and one-way ANOVA with Tukey's post-test in G, J.

FIG. 7: The mevalonate pathway controls proliferation in vivo in mice.
A. Schematic overview of neonatal mouse experiments (panels B-D).
B. Heartsize. n=7.
C. Cardiomyocyte (MLC2v) proliferation (BrdU). n=7.
D. Cardiomyocyte cross-sectional area. n=140 from 7 hearts.
E. Schematic overview of adult mouse experiments (panels F-I).
F. Striated cardiomyocyte following fix-dissociation method.
G. Representative proliferation (BrdU⁺) and cardiomyocyte (MLC2v) staining.
H. DNA synthesis (BrdU⁺) in adult cardiomyocytes (MLC2v) in vivo. n=5-6 mice.
I. DNA synthesis (BrdU⁺) in adult cardiomyocytes (MLC2v) in vivo. n=4-6 mice (data from H used).
   Scale bars=20 μm. Data is presented as mean±s.e.m. *, ***, denote P<0.05, P<0.001, respectively, using t-test for B,D and Kruskal-Wallis test with Dunn's post-test for H,I.

FIG. 8: Additional analysis of cell cycle induction with compound 3 and compound 65 (related to FIG. 3).

A. Bi-nucleation analysis following treatment with 3 μM compound 3 or 1 μM compound 65 after 4 days. Numbers displayed represent cardiomyocytes quantified.
B. Ki-67 intensity performed after 2 days of compound treatment followed by 5 days with no compound. n=17-18, 3 experiments.
Scale bars=20 μM. Data is presented as mean±s.e.m.

FIG. 9: Volcano plots of the RNA-sequencing and proteomics data from mature hCO treated with compounds for 2 days (related to FIGS. 4 and 5).

A. RNA-sequencing volcano plots for mature hCO treated with 3 μM compound 3, 0.3 μM compound 63, or 1 μM compound 65.
B. Proteomics volcano plots for mature hCO treated with 3 μM compound 3, 0.3 μM compound 63, or 1 μM compound 65.
C. Proteomics volcano plots for mature hCO treated with 10 μM compound 6.28, 5 CHIR99021, 1 μM compound 51, or 5 μM CHIR99021 plus 1 μM compound 51.

FIG. 10: IPA™ analysis of hCO RNA sequencing data (related to FIG. 4).

A. Compound 3 regulates a MAPK14 (p38α) driven network.
B. Compound 65 regulates a TGFBR and BMPR driven network converging on SMAD2/3.

FIG. 11: Compound 51 and XMU-MP-1 are MST1 inhibitors that can activate proliferation in mature hCO in combination with GSK3 inhibition (Related to FIG. 5).

A. Compound 6.28 inhibits both GSK3 and MST1, leading to activation of β-catenin and YAP1, respectively.
B. Chemical structure of compound 51.
C. Ki-67+ cardiomyocytes (α-actinin) in hCO. n=11 from 2 experiments.
D. Ki-67 intensity treated with 5 μM CHIR99021 and/or 1 μM compound 51 for 1 day. n=9-11, 2 experiments.
E. Ki-67 intensity following CHIR99021 and compound 51 or 1 μM XMU-XP-1 treatment for after 2 days. n=4-5.
F. Force of contraction with different GSK3 or MST1 inhibitors. n=4-29 hCO.
G. Force does not recovery following treatment with 5 μM of CHIR99021 for 2 days. n=8, 2 experiments.
H. Force does not recovery following treatment with 10 μM of compound 6.28 for 2 days. n=9, 2 experiments.
I. Conditions and numbers of hCO for the proteomic assessment of GSK3i and MSTli effects and synergy.
Scale bars=20 μm Data is presented as mean±s.e.m. **, ***, ****, denotes P<0.01, P<0.001, P<0.0001 respectively, using t-test in C, one-way ANOVA with Dunnett's post-test relative to DMSO in D,F and day 0 in G,H or Tukey's post-test in E.

FIG. 12: The different compounds activate different cell cycle proteins (Related to FIG. 5).

Note the lack/lower level of induction in many cell cycle proteins with compound 63 treatment in comparison to compound 3 and compound 65 in the lower half of the heat-map.

FIG. 13: Automated image cytometry of 2D hPSC-CM cultures for proliferation and cardiomyocyte size for high throughput analysis (Related to FIG. 6).

A. Image cytometry quality control outputs. DNA staining overlaid with red dots on each nuclei to ensure each nuclei has been counted once. Ki-67 staining overlaid with red dots on each nuclei to ensure each Ki-67 nuclei has been counted. Red nuclei dots overlaid on α-actinin staining to ensure no non-myocytes are counted as cardiomyocytes. α-actinin thresholding to ensure that it matches the α-actinin staining. Cytometry plot on the right is the intensity of Ki-67 positive cells (y-axis) versus intensity of α-actinin positive cells on a log 2 scale. Gates a drawn to ensure and quality control is checked and then this is applied to the image batch in an automated manner. Numbers in the gates are percentages.
B. Cardiomyocyte number following treatment of cardiomyocytes cultured in 2D following just 1 day of treatment with simvastatin +/− other metabolites. n=12 from 3 experiments.
C. Cardiomyocyte size following treatment of cardiomyocytes cultured in 2D following just 1 day of treatment with simvastatin +/− other metabolites. n=12 from 3 experiments.
D. Treatment of mature hCO with 3 μM compound 3 decreases cardiomyocyte size with no additional effects caused by 10 μM simvastatin after 2 days. n=5-6.
E. Treatment of mature hCO with 1 μM compound 65 combined with 10 μM simvastatin leads to increased cardiomyocyte size after 2 days. n=11-12, 2 experiments.
Data is presented as mean±s.e.m. ** denotes P<0.01, using Mann-Whitney F, one-way ANOVA with Dunnett's post-test relative to CTRL for C or Tukey's post-test D,E. Mev—mevalonate, GGPP—geranylgeranyl pyrophosphate, FPP—farsenyl pyrophosphate.

FIG. 14: Activation of YAP/TAZ following treatment of hCO with different compounds (Related to FIG. 5 and Discussion).

A. Activation of classical (CTGF and AXL) or cell cycle orientated (AURKB) YAP/TAZ target genes under different conditions from the hCO proteomics data.
B. Immunostaining of YAP in hCO following treatment with the different compounds. Data is presented as mean±s.e.m. * indicates p LIMMA <0.05 for compound 3, 63 and 65 and FDR <0.10 for compound 51, CHIR99021, compound 51+CHIR99021 and compound 6.28.

FIG. 15: The mevalonate pathway regulates cardiomyocyte proliferation.

a) The mevalonate pathway and associated enzymes. Note that the key regulated enzymes in that are critical for the core regeneration pathway (HMGCS1, MVD) and shuttling of metabolites out of the pathway (CYP51A, SQLE).
b) Addition of mevalonate directly controls the cell cycle
c) NAP1L4 can be farnesylated
d) Compound 3 and 65 increase NAP1L1 Cys-388 farnesylation. n=5.
e) Only compound 65 increases NAP1L4 Cys-383 farnesylation. n=5.
Scale bars=20 μm. Data is presented as mean±s.e.m. *, **, ***, **** denote P<0.05, P<0.01, P<0.001, and P<0.0001 respectively, using on-way ANOVA with Tukey's post-test in b and Dunnett's post-test relative to DMSO in f,h,i.

### DETAILED DESCRIPTION

The present invention has arisen from work that utilized human pluripotent stem cell (PSC)-cardiac organoids to screen for pro-proliferative compounds thereof. One aspect of this work was the discovery of a proliferation signature that was associated with activation or upregulation of sterol biosynthesis and, more particularly, the mevalonate pathway. The present invention is therefore directed to a composition and/or method for facilitating proliferation of mature or adult cardiomyocytes in vitro as well as in vivo in a subject.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

As used herein, except where the context requires otherwise, the term “comprise” and variations of the term, such as “comprising”, “comprises” and “comprised”, are not intended to exclude further additives, components, integers or steps.

It will be appreciated that the indefinite articles “a” and “an” are not to be read as singular indefinite articles or as otherwise excluding more than one or more than a single subject to which the indefinite article refers. For example, “a” cell includes one cell, one or more cells and a plurality of cells.

As used herein, the term “about” qualifies a stated value to encompass a range of values above or below the states value. Preferably, in this context the range may be 2, 5 or 10% above or below the stated value. By way of example only, “about 100 μM” may be 90-110 μM, 95-105 μM or 98-102 μM.

For the purposes of this invention, by “isolated” is meant material that has been removed from its natural state or otherwise been subjected to human manipulation. Isolated material (e.g., cells) may be substantially or essentially free from components that normally accompany it in its natural state, or may be manipulated so as to be in an artificial state together with components that normally accompany it in its natural state.

By “enriched” or “purified” is meant having a higher incidence, representation or frequency in a particular state (e.g., an enriched or purified state) compared to a previous state prior to enrichment or purification.

In certain aspects, the invention is broadly directed to a method and/or composition suitable for inducing or stimulating the proliferation of adult or mature cardiac cells, such as cardiomyocytes, in vitro or in vivo.

An aspect of the invention relates to a method of promoting, facilitating or inducing cardiomyocyte proliferation in vitro, the method including the step of contacting one or a plurality of cardiomyocytes with an effective amount of an agent capable of at least partly activating sterol biosynthesis therein to thereby induce cardiomyocyte proliferation.

A further aspect of the invention provides one or more cardiomyocytes or cardiac tissues or organoids comprising same, produced by the above method.

A related aspect provides a method of promoting, facilitating or inducing cardiomyocyte proliferation in a subject, the method including the step of administering to the subject an effective amount of an agent capable of at least partly activating sterol biosynthesis in a cardiomyocyte to thereby induce cardiomyocyte proliferation in the subject.

A further related aspect provides a method of treating or repairing cardiac damage or regenerating a cardiac tissue in a subject in need thereof, the method including the step of administering to the subject a therapeutically effective amount of an agent capable of at least partly activating sterol biosynthesis in cardiomyocytes to thereby treat or repair the cardiac damage in the subject, wherein the agent is preferably capable of promoting or inducing cardiomyocyte proliferation in the subject.

As used herein “cardiomyocytes” are cardiac muscle cells also known as myocardiocytes or cardiac myocytes, that make up cardiac muscle such as found in the atria and ventricles of the heart. Each myocardial cell contains myofibrils, which are the fundamental contractile units of cardiac muscle cells. Cardiomyocytes typically contain one or two nuclei, although they may have as many as four and a relatively high mitochondrial density, facilitating production of adenosine triphosphate (ATP) for muscle contraction. Myocardial infarction causes the death of cardiomyocytes. In adults, the heart's limited capacity to regenerate these lost cardiomyocytes leads to compromised cardiac function and high morbidity and mortality. In this regard, adult mammalian cardiomyocytes are considered terminally differentiated and generally incapable of proliferation. To this end, the present invention provides methods of cardiac regeneration through cardiomyocyte proliferation.

The term refers to cardiomyocytes of any species including mammalian (e.g., human) at any stage of development. According to a specific embodiment, the cardiomyocyte is a neonatal cardiomyocyte (e.g., for humans, up 6 months after birth). According to a specific embodiment, the cardiomyocyte is an adult cardiomyocyte (e.g., for human at least 16-18 years after birth). For some embodiments, the cardiomyocytes are of a subject having a cardiac disease, disorder or condition, as described hereinafter. In other embodiments, the cardiomyocytes are from, such as derived, isolated or purified, a healthy donor subject.

It will be appreciated that the cardiomyocytes may be naturally occurring, such as derived from a biopsy or post-mortem sample or alternatively may have been ex-vivo differentiated into cardiomyocytes (e.g., from pluripotent stem cells e.g., embryonic stem cells (hESCs) and induced pluripotent stem cells (iPSCs)). Methods of differentiating stem cells into cardiomyocytes are well known in the art.

Accordingly, in certain embodiments, the cardiomyocytes have been differentiated from progenitor cells. The progenitor cells may be, or comprise, human embryonic stem cells or induced pluripotent stem cells. To this end, it will be appreciated that the method of the first mentioned aspect may be used to generate or grow iPSC-derived or hESC-derived cardiomyocytes in vitro. Such cardiomyocytes may be useful for a range of in vitro and in vivo applications as are known in the art, such as those hereinafter described.

It is contemplated that cardiomyocytes produced by the first mentioned aspect may be suitable for producing cardiac cell suspensions, monolayers or “2D cultures”. In other particular embodiments, the cardiomyocytes may be suitable for producing cardiac muscle tissue in three dimensional (3D) structures such as EHT or cardiac “organoids”. Organoids may be used for producing engineered or artificial cardiac tissue. For example, cardiac organoids may be incorporated within a scaffold, such as a decellularised human heart, polyester fleece or biodegradable polymer scaffold, to thereby produce a cardiac 3D structure. Also contemplated are “bioprinted” 3D cardiac structures.

It will also be appreciated that the aforementioned method may provide potential sources of purified, differentiated cardiomyocytes for cellular therapy of the heart. In a particular embodiment, iPSC lines derived, obtained or originating from a patient with a genetic cardiac defect or disease may be used for repair of genetic mutation(s) in vitro. Such cardiomyocytes (or EHT or organoids thereof), could be administered to a patient for autologous cellular therapy. In some embodiments, cardiomyocytes (or EHT or organoids thereof) produced according to the invention may be administered directly to the heart in the form of a tissue patch, mat, plug, bolus or other implantable form.

It will also be appreciated that the cardiomyocytes and/or cardiac organoids described herein may provide potential sources of purified, differentiated cardiomyocytes for cardiac disease modelling and cardiac biology, such as modelling, investigating or predicting the effects of modulating gene expression (e.g gene “knock out”, “knock-down” or overexpression).

Further, it is envisaged that the cardiomyocytes and/or cardiac organoids described herein may be useful in monitoring the effect of one or more molecules thereon, such as for toxicity screening or for in vitro drug safety testing.

As used herein the term “promoting, facilitating or inducing cardiomyocyte proliferation” refers to an increase in cardiomyocyte proliferation which is statistically significant (as compared to untreated cells of the same origin and developmental stage) and is a result of contacting the cardiomyocytes with the agent described herein.

As used herein the phrase “cardiac regeneration” refers to the ability to trigger regeneration of heart muscle (e.g., in a pathologic state (traumatic, chronic or acute)). In other words, cardiac regeneration much depends on the induction of proliferation of cardiomyocytes.

Accordingly, this cardiac regeneration may be used to treat or repair existing cardiac damage in a subject. In this regard, the subject may have or is at risk of developing a cardiac disease, disorder or condition selected from the group consisting of a myocardial infarction, a congestive heart failure, tachyarrhythmia, familial hypertrophic cardiomyopathy, ischemic heart disease, idiopathic dilated cardiomyopathy, congenital heart disease (e.g., hypoplastic heart disease) and myocarditis.

Methods of treating cardiac damage may be prophylactic, preventative or therapeutic and suitable for treatment of cardiac damage in mammals, particularly humans. As used herein, “treating”, “treat” or “treatment” refers to a therapeutic intervention, course of action or protocol that at least ameliorates a symptom of cardiac damage after the cardiac damage and/or its symptoms (e.g., cardiomyocyte loss, fibrosis) have at least started to develop. As used herein, “preventing”, “prevent” or “prevention” refers to therapeutic intervention, course of action or protocol initiated prior to the onset of cardiac damage and/or a symptom of cardiac damage (e.g., cardiomyocyte loss, fibrosis) so as to prevent, inhibit or delay or development or progression of the cardiac damage or the symptom thereof. Those of skill in the art will understand that various methodologies and assays can be used to assess the development of a symptom or pathology of cardiac damage, and similarly, various methodologies and assays may be used to assess the reduction, remission or regression of a symptom or pathology of cardiac damage.

As used herein, the term “agent” refers to a substance which can be of a biological nature (e.g., a proteinaceous substance, such as a polypeptide/peptide or an antibody, a nucleic acid molecule, such as a polynucleotide or an oligonucleotide, or a chemical, such as a small molecule). Given its role, the agent may be referred to as an activator of sterol biosynthesis or sterol biosynthesis activator.

The skilled person would appreciate that the sterol biosynthesis pathway, also known as the cholesterol biosynthesis pathway, refers to that biological pathway involved in the synthesis of sterols (i.e., steroid alcohols, such as cholesterol) which typically are components of cell membranes in plants, animals and fungi.

The carbon skeleton of a sterol molecule is initially derived from acetyl-CoA, with the exception to the presence of the C24 methyl group in the ergosterol side chain. The first reactions in the sterol biosynthetic pathway involve condensation of two acetyl-CoA units to form acetoacetyl-CoA, followed by the addition of a third unit to form 3-hydroxy-3-methylglutaryl-CoA (HMG-CoA), which is then reduced by NADPH to give mevalonic acid. These initial steps of the sterol biosynthesis pathway constitute the mevalonate pathway.

As such, the term “mevalonate pathway” or “mevalonate biosynthesis” is used herein to refer to that portion of the sterol biosynthetic pathway that converts acetyl-CoA to isopentenyl pyrophosphate. The mevalonate pathway comprises enzymes that catalyze the following steps: (a) condensing two molecules of acetyl-CoA to acetoacetyl-CoA; (b) condensing acetoacetyl-CoA with acetyl-CoA to form HMG-CoA; (c) converting HMG-CoA to mevalonate; (d) phosphorylating mevalonate to mevalonate 5-phosphate; (e) converting mevalonate 5-phosphate to mevalonate 5-pyrophosphate; and (f) converting mevalonate 5-pyrophosphate to isopentenyl pyrophosphate. As would be appreciated by the skilled person, these mevalonate pathway enzymes may include acetoacetyl-CoA thiolase, HMG-CoA synthase, HMG-CoA reductase, mevalonate-5-kinase, phosphomevalonate kinase and mevalonate pyrophosphate decarboxylase. In certain embodiments, the isopentenyl pyrophosphate isomerase, which converts isopentenyl pyrophosphate (IPP) into dimethylallyl pyrophosphate (DMAPP), is also referred to as a mevalonate pathway enzyme.

After the mevalonate pathway, the next steps constitute the isoprenoid pathway. Isoprenoids are the most diverse and abundant compounds present in nature, and are essential components of all organisms due to a variety of roles in different biological processes. First, mevalonate is converted to isopentenyl diphosphate (IPP) by two phosphorylation reactions followed by one decarboxylation. Subsequently, isomerization of IPP by isopentenyl diphosphate isomerase produces dimethylallyl diphosphate (DMAPP). After that, longer isoprenoids are formed by a consecutive condensation of IPP with DMAPP and geranyl diphosphate (GPP) to produce the 15-carbon isoprenoid compound known as farnesyl diphosphate (FPP) in two reactions catalyzed by the enzyme farnesyl diphosphate synthase (FPPS). All these reactions together constitute the isoprenoid pathway.

After the isoprenoid pathway, the next two reactions comprise the first committed step in sterol biosynthesis. These are catalyzed by the enzyme squalene synthase, which promotes a head-to-head condensation of two molecules of farnesyl diphosphate to produce squalene. In the first reaction, presqualene pyrophosphate (PPP) is produced by the loss of an inorganic pyrophosphate. This is converted to squalene in the second reaction in presence of NADPH, an essential cofactor required to drive this conversion.

After production of squalene, sterol biosynthesis continues with the synthesis of 2,3-oxidosqualene (or squalene epoxide) in a reaction catalyzed by the enzyme squalene epoxidase (or squalene monooxygenase). 2,2-oxidosqualene cyclase then cyclizes the intermediate 2,3-oxidosqualene to lanosterol, the initial precursor of all steroid structures formed by mammals, fungi, and trypanosomatids. Several sequential transformations by a number of enzymes then occur to form cholesterol in mammals.

Upregulation or activation of sterol biosynthesis, inclusive of the mevalonate pathway and the isoprenoid pathway, can be effected at the activity or expression level of one or more of the components (e.g., enzymes) thereof at the genomic level, at the transcript level or at the protein level.

In particular embodiments, activation or upregulation of sterol biosynthesis, inclusive of mevalonate biosynthesis and isoprenoid biosynthesis, by the agent described herein comprises, at least in part, modulating, and more particularly increasing, the expression and/or activity of one or more proteins or enzymes of, or associated with sterol biosynthesis, inclusive of the mevalonate pathway and the isoprenoid pathways, such as those described above.

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., phosphoproteins).

Also provided are protein “variants” such as naturally occurring (eg allelic variants) and orthologs. Preferably, protein variants share at least 70% or 75%, preferably at least 80% or 85% or more preferably at least 90%, 91%, 92%, 93%, 94%, 95%, 96%, 97%, 98% or 99% sequence identity with an amino acid sequence disclosed herein.

Also provided are protein fragments, inclusive of peptide fragments that comprise less than 100% of an entire amino acid sequence. In particular embodiments, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375 and 400 contiguous amino acids of said protein.

A “peptide” is a protein having no more than fifty (50) amino acids.

A “polypeptide” is a protein having more than fifty (50) amino acids.

As would be appreciated by the skilled artisan, the expression level of one or more proteins described herein, inclusive of enzymes, may include one or more phosphorylated forms of said proteins (i.e., a phosphoprotein).

In specific embodiments, the one or more proteins and/or enzymes are selected from the group consisting of squalene monooxygenase (SQLE), Hydroxymethylglutaryl(HMG)-CoA synthase (HMGCS1), Lanosterol 14 alpha-demethylase (CYP51A1), HMG-CoA reductase (HMGCR), Hydroxymethylglutaryl(HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase (e.g., geranylgeranyltransferase I and rab geranylgeranyltransferase), a farnesyltransferase, Sterol regulatory element-binding protein 1 (SREBP1), Sterol regulatory element-binding protein 2 (SREBP2) and any combination thereof.

In this regard, the protein of or associated with sterol biosynthesis can be or comprise a transcription factor that at least partly controls cholesterol homeostasis by stimulating transcription of sterol biosynthesis regulated or related genes (e.g., Sterol regulatory element-binding protein 1 (SREBP-1) also known as sterol regulatory element binding transcription factor 1 (SREBF1) and Sterol regulatory element-binding protein 2 (SREBP-2) also known as sterol regulatory element binding transcription factor 2 (SREBF2)).

In some embodiments, activation or upregulation of sterol biosynthesis, inclusive of mevalonate biosynthesis and isoprenoid biosynthesis, by the agent described herein comprises, at least in part, modulating, and more particularly increasing, the prenylation, such as the farnesylation and/or geranylgeranylation, of one or more proteins or peptides, such as a cell cycle protein as hereinbefore described.

By way of example, prenylation of GTP-binding proteins, which regulate F-actin formation and cell cycle protein stability (e.g., YAP1/TAZ stability) may result in activation of pro-proliferative pathways. Metabolism through Coenzyme Q may also be important in this regard as it is derived directly from geranylgeranyl pyrophosphate. Autophagy may also be controlled by prenylation of one or more cell cycle proteins (Miettinen and Bjorklund, 2015).

Prenylation (also referred to as isoprenylation or lipidation) is a post-translational modification of proteins by which hydrophobic molecules, such as an isoprenyl group (e.g., farnesyl group, geranylgeranyl group), are post-translationally added to a protein or chemical compound typically by a prenyltransferase (e.g., a farnesyl transferase, a geranylgeranyl transferase).

Geranylgeranylation is a form of prenylation. The term “geranylgeranylation” refers to the attachment of a 20-carbon lipophilic geranylgeranyl isoprene unit to a cysteine amino acid residue typically located at the C-terminus of a protein. The geranyl-geranyl group is typically attached through a thioether bond to a cysteine residue.

Farnesylation is a further type of prenylation. The term “farnesylation” refers to the addition of a farnesyl group to peptides or proteins typically bearing a CaaX or CxxM motif (i.e., a four-amino acid sequence at the carboxyl terminus of the peptide or protein). The farnesyl group is generally a 15-carbon isoprenoid lipid.

In particular embodiments, the agent of the invention is co-administered with an isoprenoid, such as mevalonate, geranylgeranyl, farnesyl (and/or one or more derivatives or precursors thereof) and any combination thereof.

In particular embodiments, the agent is administered to elicit an upregulation in activity of sterol biosynthesis, such as the mevalonate pathway and/or the isoprenoid pathway, in a transient manner until appearance of hypertrophic, regenerative or hyperplastic effects of increasing cardiomyocyte proliferation.

As used herein, the terms “therapeutically effective amount” or “effective amount” describe a quantity of a specified agent (e.g., an agent capable of at least partly activating a mevalonate pathway in cardiomyocytes) sufficient to achieve a desired effect in a subject being treated with that agent. For example, this can be the amount of a composition comprising the agent capable of at least partly activating a mevalonate pathway in cardiomyocytes that is necessary to inducing cardiomyocyte proliferation, treat or repair cardiac damage and/or regenerate a cardiac tissue in the subject. In some embodiments, a “therapeutically effective amount” is sufficient to reduce or eliminate a symptom of cardiac damage or a cardiac disease, disorder or condition.

Ideally, a therapeutically effective amount of an agent is an amount sufficient to induce the desired result without causing a substantial cytotoxic effect in the subject. The effective amount of the agent capable of activating a mevalonate pathway will be dependent on the subject being treated, the type and severity of any associated disease, disorder and/or condition (e.g., the severity of cardiac damage), and the manner of administration of the therapeutic composition.

Suitably, the agent described herein is administered to a subject as a pharmaceutical composition comprising a pharmaceutically-acceptable carrier, diluent or excipient. In this regard, any dosage form and route of administration, such as those provided therein, may be employed for providing a subject with the composition of the invention.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In specific embodiments, one may administer the agent or pharmaceutical composition in a local rather than systemic manner, for example, via injection of the pharmaceutical composition directly into a tissue region of a patient. For example, by direct intraventricular or intracardiac injections (e.g., into the right or left ventricular cavity, into the common coronary artery). Also contemplated is administration of the composition directly to the myocardium (e.g., either during open heart surgery or endomyocardial catheters guided by imaging, such as ultrasound). Additionally, it is envisaged that the agents as described herein can be immobilized to an implant or implantable device (e.g., stent, mesh, synthetic graft) where they can be slowly released (or sustained released) therefrom.

Suitably, an agent which upregulates or activates sterol biosynthesis, inclusive of the mevalonate pathway and the isoprenoid pathway, such as an enzyme thereof, modulates a signalling effector upstream or downstream of the sterol biosynthesis pathway. In particular embodiments, the signalling effector is selected from the group consisting of p38α, MST1, a TGF-beta receptor, a BMP receptor and any combination thereof. To this end, the agent can be or comprises a p38α inhibitor, a MST1 inhibitor, a TGF-beta receptor inhibitor and/or a BMP receptor inhibitor.

In alternative embodiments, the agent is not a p38α inhibitor (e.g., agent has an IC50 in relation to the kinase activity of p38α that is greater than about 250 nm, 500 nm or 1000 nm) or a MST1 inhibitor (e.g., agent has an IC50 in relation to the kinase activity of MST1 that is greater than about 250 nm, 500 nm or 1000 nm).

p38 mitogen-activated protein kinases are a class of mitogen-activated protein kinases that are responsive to stress stimuli, such as cytokines, ultraviolet irradiation, heat shock, and osmotic shock, and are involved in cell differentiation, apoptosis and autophagy. Four p38 MAP kinases, p38-α (MAPK14), -β (MAPK11), -γ (MAPK12/ERK6), and -δ (MAPK13/SAPK4), have been identified. For the present invention, the p38α inhibitor is suitably specific or selective to the alpha isoform of p38 and preferably has little or no off target effect on the remaining beta, gamma and delta isoforms of p38. In alternative embodiments, the p38α inhibitor is or comprises a dual inhibitor of p38α and p38β.

Suitably, a p38α inhibitor inhibits p38α in vitro with an IC₅₀ of less than 1 μm, 0.5 μm or 0.25 μm as determined by, for example, an assay, such as a kinase assay, described herein.

Suitable inhibitors of p38α include, but are not limited to, BIRB 796 (Doramapimod), Skepinone-L, LY2228820, TAK-715, VX-745, VX-702, PH-797804, SB239063 and SB203580.

Mammalian STE20-like kinase 1 (MST1) is a component of the “Hippo” signalling pathway, and has been implicated in regulating the cell cycle, apoptosis and cellular responses to oxidative stress (see, e.g., Choi, J., et al., Plos One 4(11):e8011, 1 (2009)). MST1 is also known as serine/threonine kinase 4 (STK4) and kinase responsive to stress 2 (KRS2). These terms may be used interchangeably, and include as well variants, isoforms, species homologs of human or murine MST1, and analogues having at least one common epitope with human or murine MST1. The term also includes all the physiologically relevant post-translational chemical modifications forms, for example, glycosylation, phosphorylation or acetylation, etc., provided that the functionality of the protein is maintained. An MST1 inhibitor may include, for example, a small molecule, an antibody or an siRNA.

The MST1 inhibitor may be any type of compound. For example, the compound may be a small organic molecule or a biological compound such as an antibody or an enzyme. To this end, a person skilled in the art may be able to determine whether a compound is capable of inhibiting MST1 activity and/or expression by any means known in the art.

In particular embodiments, the MST1 inhibitor is also capable of or configured to inhibit the closely related MST2 kinase (i.e., Serine/threonine-protein kinase 3; STK3). Accordingly, the agent of the invention may be a dual MST1/MST2 inhibitor.

Suitably, an MST1 inhibitor inhibits MST1 in vitro with an IC₅₀ of less than 1 μm, 0.5 μm or 0.25 μm as determined by, for example, an assay, such as a kinase assay, described herein.

The term “TGF-beta receptor” or “TGFβR” is used herein to encompass all three sub-types of the TGFβR family (i.e., TGFβR-1, TGFβR-2, TGFβR-3). The TGFβ receptors are characterized by serine/threonine kinase activity and exist in several different isoforms that can be homo- or heterodimeric.

As used herein, the term “TGF-β signalling pathway” is used to describe the downstream signalling events attributed to TGF-β and TGF-β like ligands. For example, in one signalling pathway a TGF-β ligand binds to and activates a Type II TGF-β receptor. The Type II TGF-β receptor recruits and forms a heterodimer with a Type I TG-β receptor. The resulting heterodimer permits phosphorylation of the Type I receptor, which in turn phosphorylates and activates a member of the SMAD family of proteins (e.g., Smad 2, Smad 3). A signalling cascade is triggered, which is well known to those of skill in the art, and ultimately leads to control of the expression of mediators involved in cell growth, cell differentiation, tumorigenesis, apoptosis, and cellular homeostasis, among others. Other TGF-β signalling pathways or components thereof are also contemplated for manipulation according to the methods described herein.

The term “inhibitor of the TGF-β signalling pathway” as used herein, refers to inhibition of at least one of the proteins involved in the signal transduction pathway for TGF-β. It is contemplated herein that an inhibitor of the TGF-β signalling pathway can be, for example, a TGF-β receptor inhibitor (e.g., a small molecule, an antibody, an siRNA), a TGF-βsequestrant (e.g., an antibody, a binding protein), an inhibitor of receptor phosphorylation, an inhibitor of a SMAD protein, or a combination of such agents.

Suitably, a TGF-β receptor inhibitor inhibits a TGF-β receptor in vitro with an IC₅₀ of less than 1 μm, 0.5 μM or 0.25 μm as determined by, for example, an assay, such as a kinase assay, described herein.

The term “bone morphogenic protein (BMP) receptor” as used herein refers to a group of receptors that bind cytokines forming part of the transforming growth factor-B (TGF-β) superfamily. In this regard, BMP ligands bind to a complex of the BMP receptor type II and a BMP receptor type I (Ia or Ib). This leads to the phosphorylation of the type I receptor that subsequently phosphorylates the BMP-specific Smads (Smad1, Smad5, and Smad8), allowing these receptor-associated Smads to form a complex with Smad4 and move into the nucleus where the Smad complex binds a DNA binding protein and acts as a transcriptional enhancer.

Similar to the above, it is contemplated herein that an inhibitor of the BMP signalling pathway can be, for example, a BMP receptor inhibitor (e.g., a small molecule, such as LDN-193189, an antibody, an siRNA), a BMP sequestrant (e.g., an antibody, a binding protein), an inhibitor of BMP receptor phosphorylation, an inhibitor of a SMAD protein, or a combination of such agents.

Suitably, a BMP receptor inhibitor inhibits a BMP receptor in vitro with an IC₅₀ of less than 1 μm, 0.5 μM or 0.25 μm as determined by, for example, an assay, such as a kinase assay, described herein.

In embodiments relating to antibody inhibitors, the antibody may be polyclonal or monoclonal, native or recombinant. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative of the protein product of p38α, MST1, a TGF-beta receptor and/or a BMP receptor. For example, the antibodies may be polyclonal antibodies. Such antibodies may be prepared for example by injecting an isolated protein, fragment, variant or derivative of the protein in question into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated p38α, MST1, a TGF-beta receptor and/or a BMP receptor protein products and/or fragments, variants and/or derivatives thereof.

In particular embodiments, the agent is further capable of at least partly modulating (i.e., increasing or decreasing) the expression and/or activity of a cell cycle protein.

The term “cell cycle protein” refers to a protein whose expression and/or activity level closely tracks the progression of the cell through the cell-cycle (see, e.g., Whitfield et al., Mol. Biol. Cell (2002) 13:1977-2000). More specifically, cell cycle proteins (and their corresponding cell cycle genes) show periodic increases and decreases in expression that coincide with certain phases of the cell cycle (e.g., STK15 and PLK show peak expression at G2/M). Certain cell cycle proteins have clear, recognized cell-cycle related function, such as DNA synthesis or repair, chromosome condensation, or cell-division. Some cell cycle proteins, however, have expression levels that track the cell-cycle without having an obvious, direct role in the cell-cycle and, as such, need not have a recognized role in the cell-cycle. Exemplary cell cycle proteins and their encoding genes are listed in International Application No. PCT/US2010/020397 (pub. no. WO/2010/080933) (see, e.g., Table 1 in WO/2010/080933). International Application No. PCT/US2010/020397 (pub. no. WO/2010/080933 (see also corresponding U.S. application Ser. No. 13/177,887)) and International Application No. PCT/US2011/043228 (pub no. WO/2012/006447 (see also related U.S. application Ser. No. 13/178,380)) and their contents are hereby incorporated by reference in their entirety.

According to specific embodiments, the cell cycle protein is selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

It will be appreciated that determining the expression of a cell cycle protein may include determining one or both of the nucleic acid levels thereof, such as by nucleic acid amplification and/or nucleic acid hybridization, and/or the protein levels thereof.

The terms “determining”, “measuring”, “evaluating”, “assessing” and “assaying” are used interchangeably herein and may include any form of measurement known in the art, such as those described herein.

Determining, assessing, evaluating, assaying or measuring nucleic acids of a cell cycle protein, such as RNA, mRNA and cDNA, may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a cell or tissue source. In other embodiments, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

Determining, assessing, evaluating, assaying or measuring protein levels of a cell cycle protein may be performed by any technique known in the art that is capable of detecting cell- or tissue-expressed proteins whether on the cell surface or intracellularly expressed, or proteins that are isolated, extracted or otherwise obtained from the cell of tissue source. These techniques include antibody-based detection that uses one or more antibodies which bind the protein, electrophoresis, isoelectric focusing, protein sequencing, chromatographic techniques and mass spectroscopy and combinations of these, although without limitation thereto. Antibody-based detection may include flow cytometry using fluorescently-labelled antibodies that bind a cell cycle protein, ELISA, immunoblotting, immunoprecipitation, in situ hybridization, immunohistochemistry and immunocytochemistry, although without limitation thereto. Suitable techniques may be adapted for high throughput and/or rapid analysis such as using protein arrays such as a TissueMicroArray™ (TMA), MSD MultiArrays™ and multiwell ELISA, although without limitation thereto.

As would be understood by the skilled person, the expression level of the one or more proteins and/or enzymes of, or associated with, sterol biosynthesis and/or the cell cycle proteins is deemed to be “altered” or “modulated” when the amount or expression level of the respective enzyme or protein is increased or up regulated or decreased or down regulated, as defined herein.

By “enhanced”, “increased” or “up regulated” as used herein to describe the expression level of an enzyme or a protein, refers to the increase in and/or amount or level thereof, including variants, in a biological sample when compared to a control or reference sample or a further biological sample from a subject. The expression level thereof may be relative or absolute. In some embodiments, the expression level of the enzyme or protein is increased if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% higher than the level of expression of the corresponding enzyme or protein in a control sample or further biological sample from a subject.

The terms, “reduced” and “down regulated”, as used herein to describe the expression level of an enzyme or a protein, refer to a reduction in and/or amount or level thereof, including variants, in a biological sample when compared to a control or reference sample or further biological sample from a subject. The expression level thereof may be relative or absolute. In some embodiments, the expression level of the enzyme or protein is reduced or down regulated if its level of expression is more than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level of expression of the corresponding enzyme or protein in a control sample or further biological sample from a subject.

The term “control sample” typically refers to a biological sample from a healthy or non-diseased individual. In one embodiment, the control sample may be from a subject known to be free of a cardiac disease, disorder or condition. The control sample may be a pooled, average or an individual sample. An internal control is a marker from the same biological sample being tested.

Suitably, the agent has little or no glycogen synthase kinase (GSK) inhibitory activity or is not a GSK inhibitor. The term “glycogen synthase kinase (GSK) inhibitor” as used herein refers to an agent that inhibits a GSK. It will be appreciated that GSK is a protein kinase that includes GSK 1, GSK 2 and GSK 3, inclusive of isoforms thereof, such as GSK-3α, GSK-3β and/or GSK-3β2. Preferably, the agent of the invention demonstrates minimal or no GSK-3 inhibitory activity.

In certain embodiments, the agent has an IC50 in relation to the kinase activity of GSK, and in particular that of GSK-3, of greater than about 200 nM (e.g., greater than about 200, 250, 300, 350, 400, 450, 500, 550, 600, 650, 700, 750, 800, 850, 900, 900, 950, 1000 nM and any range therein), preferably greater than about 500 nM and more preferably greater than about 1 μM.

Suitably, the agent maintains, at least in part, contractile function of proliferated cardiomyocytes. This may be relative to the contractile function of a normal or standard healthy cardiomyocyte or a cardiomyocyte from which the proliferated cardiomyocytes are derived from.

Determining the contractile function of proliferated or proliferative cardiomyocytes that, for example, comprise a portion of regenerated cardiac tissue may include any known in the art. By way of example, this may comprise determining the expression of one or more sarcomere proteins and/or gap junction proteins, such as those described herein. Additionally, methods to directly or indirectly assess the force of contraction of the proliferated cardiomyocytes may be utilized to determine their contractile function, such as per those methods described in the below Example or via organ baths containing force transducers (see, e.g., Zimmermann et al. Circulation Research, 2002).

Contractile function may be further ascertained by detecting responsiveness to pharmacological agents such as beta-adrenergic agonists (e.g., isoprenaline), adrenergic beta-antagonists (e.g., esmolol), cholinergic agonists (e.g., carbochol), and the like.

Alternatively or additionally, validating the contractile nature of the cardiomyocytes can be achieved by detecting electrical activity and/or calcium transients of the cells. Electrical activity and calcium transients can be measured by various methods, including extracellular recording, intracellular recording (e.g., patch clamping), and use of voltage-sensitive dyes. Such methods are well known to those skilled in the art.

Suitably, the cardiomyocytes of the first mentioned aspect and the subject of the second and third mentioned aspects are not to be co-treated with or co-administered an inhibitor of sterol biosynthesis, such as an inhibitor of the isoprenoid and/or mevalonate pathways. By way of example, known inhibitors of sterol biosynthesis include statins (i.e., HMG-CoA reductase inhibitors e.g., simvastatin, rosuvastatin, cerivastatin, fluvastatin, atorvastatin etc) and bisphosphonates (i.e., farnesyl pyrophosphate synthase inhibitors e.g., pamidronate, zoledronate). Additionally, squalene synthase inhibitors are also known in the art, such as zaragozic acid A.

In a further aspect, the invention provides a composition for use in regenerating a cardiac tissue in a subject, the composition comprising a therapeutically effective amount of an agent that activates or upregulates sterol biosynthesis in a cardiomyocyte, such as mevalonate biosynthesis and/or isoprenoid biosynthesis, and optionally a pharmaceutically-acceptable carrier, diluent or excipient.

Suitably, the present composition is for use in the methods of the aforementioned aspects. In this regard, it will be appreciated that the present composition may be used for the treatment or repair of cardiac damage in a subject.

Suitably, the agent is that hereinbefore described.

In yet a further aspect, the inventions provides a kit for use in promoting, facilitating or inducing cardiomyocyte proliferation in vitro, the kit comprising an agent capable of least partly activating sterol biosynthesis in a cardiomyocyte to thereby induce proliferation thereof.

Suitably, the present kit is for use in the method of the first mentioned aspect.

Suitably, the agent is that hereinbefore described.

In a related aspect, the invention relates to use of an agent capable of at least partly activating sterol biosynthesis, such as mevalonate biosynthesis and/or isoprenoid biosynthesis, in a cardiomyocyte, in the manufacture of a medicament for regenerating a cardiac tissue in a subject.

Suitably, the agent is capable of inducing cardiomyocyte proliferation.

In another aspect, the invention provides a method of screening, designing, engineering or otherwise producing an agent for inducing cardiomyocyte proliferation, said method including steps of:

(a) contacting one or a plurality of cardiomyocytes with a candidate molecule; and

(b) determining whether the candidate molecule is capable of at least partly activating a mevalonate pathway to thereby induce cardiomyocyte proliferation.

It will be appreciated that this aspect of the invention provides a method or system for identifying, assaying or screening candidate molecules that may modulate cardiomyocyte proliferation. Candidate molecules may be present in combinatorial libraries, natural product libraries, synthetic chemical libraries, phage display libraries, lead compound libraries and any other libraries or collections of molecules suitable for screening.

Methods of determining cardiomyocyte proliferation are well known in the art, and include, but are not limited to, manual cell counting to assess cardiomyocyte numbers (e.g., via imaging), MTT assay, genetic reporters (e.g., the FUCCI system or MADM; see, e.g., Mohamed et al., Cell 2018) and a thymidine incorporation assay. In some embodiments, the presence of proliferative cardiomyocytes is validated by confirming expression of at least one cardiomyocyte-specific marker produced by the cell. For example, the cardiomyocytes express cardiac transcription factors, sarcomere proteins, and gap junction proteins. Suitable cardiomyocyte-specific proteins include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

Nucleic acid marker expression may be detected or measured by any technique known in the art including nucleic acid sequence amplification (e.g. polymerase chain reaction) and nucleic acid hybridization (e.g. microarrays, Northern hybridization, in situ hybridization), although without limitation thereto. Protein marker expression may be detected or measured by any technique known in the art including flow cytometry, immunohistochemistry, immunoblotting, protein arrays, protein profiling (e.g 2D gel electrophoresis), although without limitation thereto. Preferably, protein markers are detected by an antibody or antibody fragment (which may be polyclonal or monoclonal) that binds the protein marker. Suitably, the antibody is labelled, such as with a radioactive label, a fluorophore (e.g Alexa dyes), digoxogenin or an enzyme (e.g alkaline phosphatase, horseradish peroxidase), although without limitation thereto.

In certain embodiments, step (b) comprises determining whether the candidate molecule activates and/or increases the expression of one or more proteins and/or enzymes of, or associated with, sterol biosynthesis. The one or more proteins and/or enzymes of, or associated with, sterol biosynthesis and methods of determining their activity and/or expression can be any as are well known in the art such as that hereinbefore described. In this regard, the one or more proteins and/or enzymes are preferably selected from the group of squalene monooxygenase (SQLE), Hydroxymethylglutaryl(HMG)-CoA synthase (HMGCS1), Lanosterol 14 alpha-demethylase (CYP51A1), HMG-CoA reductase (HMGCR), Hydroxymethylglutaryl(HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase (e.g., geranylgeranyltransferase I and rab geranylgeranyltransferase), a farnesyltransferase, sterol regulatory element binding protein 1 (SREBP1), sterol regulatory element binding protein 2 (SREBP2) and any combination thereof.

According to specific embodiments, the present method includes the further step of determining whether the candidate molecule is capable of at least partly modulating the expression and/or activity of a cell cycle protein. Again, the cell cycle protein and methods of determining their activity and/or expression can be any as are well known in the art such as that hereinbefore described. In certain embodiments, the cell cycle protein is selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

Suitably, the method of the present aspect includes the further step of determining whether the candidate molecule has little or no GSK inhibitory activity. It will be appreciated that this may be achieved by any means in the art, such as dot blots and kinase assays that measure the direct kinase activity of GSK (e.g., measures ADP formed from a kinase reaction). Preferably, the agent of the invention demonstrates minimal or no GSK-3 inhibitory activity.

It will be appreciated that the step of contacting the one or plurality of cardiomyocytes with a candidate molecule may be performed under suitable conditions, such as 2D or 3D culture, as are known in the art. In one preferred embodiment, the one or plurality of cardiomyocytes form a cardiac organoid, such as that described in International Application No. PCT/AU2017/050905, which is incorporated herein in its entirety.

In yet another aspect, the invention provides an agent for inducing cardiomyocyte proliferation screened, designed, engineered or otherwise produced according to the method of the aforementioned aspect.

Suitably, the agent is for use according to any of those methods hereinbefore described.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). Preferably, the subject is a human.

All computer programs, algorithms, patent and scientific literature referred to herein is incorporated herein by reference.

The following non-limiting examples illustrate the methods and composition of the invention. These examples should not be construed as limiting: the examples are included for the purposes of illustration only.

### EXAMPLES

### Introduction

Ninety percent of drug candidates fail to progress from Phase I trials to clinical approval (Horvath et al., 2016; Mills et al., 2018), with the majority of targets identified using current cell culture or small animal models failing to translate due to lack of efficacy (Cook et al., 2014; Mills et al., 2018). This is particularly pertinent to the cardiovascular field, where there are fewer drugs in clinical development compared with other fields of research, despite cardiovascular disease remaining the leading cause of death worldwide (Fordyce et al., 2015).

Animal models, primarily mice, are extensively used to study heart disease and offer valuable mechanistic insights. However, their extrapolation to human cardiac disease and drug safety has been poor due to considerable species differences in many functional and biological properties (Matsa et al., 2014). Human pluripotent stem cells (hPSCs) can potentially bridge this translational gap by providing an unlimited source of human cardiomyocytes for biomedical and pharmaceutical research (Matsa et al., 2014). However, hPSC-derived cardiomyocytes (hPSC-CMs) in traditional 2D culture lack functional maturation (Yang et al., 2014), which hampers their capacity to accurately predict human biology and pathophysiology in some instances (Mills et al., 2018). A potential solution to this problem are multi-cellular 3D human organoids, which provide a more accurate model (Horvath et al., 2016; Jabs et al., 2017; Mills et al., 2018; Moffat et al., 2017). In support of this notion, 3D culture systems are able to predict pharmacogenomic interactions in cancer that are undetectable in 2D assays (Jabs et al., 2017). Additionally, recent studies have shown that organoid models can predict patient outcomes in stage 1/11 clinical trials for metastatic gastrointestinal cancer (Vlachogiannis et al., 2018).

Several studies have demonstrated that 3D culture (Giacomelli et al., 2017) and 3D culture under mechanical loading enhances the maturation of hPSC-CM and facilitates modelling of disease phenotypes and drug responses (Mannhardt et al., 2016; Mills et al., 2017a; Shadrin et al., 2017; Tiburcy et al., 2017; Voges et al., 2017). Recently described is the generation of multi-cellular human cardiac organoids (hCO) derived from hPSCs, which more closely resemble native cardiac tissue architecture including highly organized cardiomyocytes and stromal cells, an endothelial capillary network and an epicardial cell layer (Mills et al., 2017a). When cultured under conditions recapitulating the postnatal metabolic environment, hCO maturation is further enhanced and there is a metabolic switch from glycolysis to fatty acid oxidation, expression of adult sarcomere isoforms, t-tubules, adult-like electrophysiological properties, extracellular matrix remodelling and cardiomyocyte cell cycle arrest (Mills et al., 2017a).

In contrast to other engineered heart tissue constructs, hCO are cultured in a 96-well format, require minimal tissue handling and allow for real-time analysis of cardiac contractile parameters, thus enabling high-content screening of mature hPSC-CM. We previously used this system to define underlying mechanisms controlling human cardiomyocyte cell cycle arrest and for predictive drug toxicology, including identification of compounds that were previously withdrawn from clinical use due to arrhythmogenic side-effects (Mills et al., 2017a).

Herein, we demonstrate hCO platforms can be used in drug discovery pipelines to identify the most efficacious activators of cardiomyocyte proliferation.

### Experimental Model and Subject Details

### Mice

Ethical approval for mouse experiments was obtained from The University of Queensland's Animal Ethics Committee (SBMS/101/13/NHMRC and SBMS/AIBN/138/16/NHMRC/NHF).

For neonatal experiments (P1), timed pregnant CD1 female mice were housed under standard conditions with 12-hour light/dark cycles and ad libitum access to food and water. For adult experiments (8 week old), CD-1 male mice were housed under standard conditions, with 12-hour light/dark cycles and ad libitum access to food and water.

### Human Pluripotent Stem Cells

Ethical approval for the use of human embryonic stem cells (hESCs) was obtained from The University of Queensland's Medical Research and QIMR Berghofer's Ethics Committee (2014000801 and P2385) and was carried out in accordance with the National Health and Medical Research Council of Australia (NHMRC) regulations. Female HES3 (WiCell) cells were maintained as TypLE (ThermoFisher Scientific) passaged cultures using mTeSR-1 (Stem Cell Technologies)/Matrigel (Millipore). Karyotyping and DNA fingerprinting were performed as a quality control.

### Method Details

### Cardiac Differentiation

Cardiac cells were produced using recently developed protocols where cardiomyocytes and stromal cells are produced in the same differentiation culture (Hudson et al., 2012; Mills et al., 2017a; Mills et al., 2017b; Voges et al., 2017); multi-cellular cultures are critical for function (Hudson et al., 2011; Tiburcy et al., 2017). hESCs were seeded at 2␣104 cells/cm2 in Matrigel-coated flasks and cultured for 4 days using mTeSR-1. They were then differentiated into cardiac mesoderm using RPMI B27-medium (RPMI1640 GlutaMAX+ 2% B27 supplement without insulin, 200 μM L-ascorbic acid 2-phosphate sesquimagnesium salt hydrate (Sigma) and 1% Penicillin/Streptomycin (all ThermoFisher Scientific unless otherwise indicated)) containing 5 ng/mL BMP-4 (RnD Systems), 9 ng/mL Activin A (RnD Systems), 5 ng/mL FGF-2 (RnD Systems) and 1 μM CHIR99021 (Stem Cell Technologies) with daily medium exchange for 3 days. Subsequently, they were specified into a hPSC-CM/stromal cell mixture using RPM B27—containing 5 μM IWP-4 (Stem Cell Technologies) followed by another 7 days of RPMI B27+(RPMI1640 GlutaMAX+2% B27 supplement with insulin, 200 μM L-ascorbic acid 2-phosphate sesquimagnesium salt hydrate and 1% Penicillin/Streptomycin) with medium exchange every 2-3 days. The differentiated cells were then cultured in RPMI B27+ until digestion at 15 days using 0.2% collagenase type I (Sigma) in 20% fetal bovine serum (FBS) in PBS (with Ca2+ and Mg2+) for 60 min at 37° C., followed by 0.25% trypsin-EDTA for 10 min. The cells were filtered using a 100 μm mesh cell strainer (BD Biosciences), centrifuged at 300×g for 3 min, and resuspended at the required density in CTRL medium: α-MEM GlutaMAX, 10% FBS, 200 μM L-ascorbic acid 2-phosphate sesquimagnesium salt hydrate and 1% Penicillin/Streptomycin. Based on flow cytometry the cells generated and used for tissue engineering were ˜70% α-actinin+/CTNT+ hPSC-CMs with the rest being predominantly CD90+ stromal cells (Voges et al., 2017), which are critical for function (Hudson et al., 2011; Tiburcy et al., 2017).

### Heart-Dyno hCO Fabrication

Heart-dyno culture inserts were fabricated using standard SU-8 photolithography and PDMS molding practices (Mills et al., 2017a). CTRL medium: α-MEM GlutaMAX (ThermoFisher Scientific), 10% fetal bovine serum (FBS) (ThermoFisher Scientific), 200 μM L-ascorbic acid 2-phosphate sesquimagnesium salt hydrate (Sigma) and 1% Penicillin/Streptomycin (ThermoFisher Scientific). For each hCO, 5×104 cardiac cells in CTRL medium were mixed with collagen I to make a 3.5 μL final solution containing 2.6 mg/mL collagen I and 9% Matrigel. The bovine acid-solubilized collagen I (Devro) was first salt balanced and pH neutralized using 10×DMEM and 0.1 M NaOH, respectively, prior to mixing with Matrigel and cells. The mixture was prepared on ice and pipetted into the Heart-Dyno. The Heart-Dyno was then centrifuged at 100×g for 10 s to ensure the hCO form halfway up the posts. The mixture was then gelled at 37° C. for 60 min prior to the addition of CTRL medium to cover the tissues (150 μL/hCO). The Heart-Dyno design facilitates the self-formation of tissues around in-built PDMS exercise poles (designed to deform ˜0.07 μm/μN). The medium was changed every 2-3 days (150 μL/hCO).

hCOs were cultured in CTRL medium for formation for 5 days and then either kept in CTRL medium culture or changed to maturation medium (Mills et al., 2017a) comprising DMEM without glucose, glutamine and phenol red (ThermoFisher Scientific) supplemented with 4% 1327—(without insulin) (ThermoFisher Scientific), 1% GlutaMAX (ThermoFisher Scientific), 200 μM L-ascorbic acid 2-phosphate sesquimagnesium salt hydrate and 1% Penicillin/Streptomycin (ThermoFisher Scientific), 1 mmol/L glucose and 100 μmon palmitic acid (conjugated to bovine serum albumin within B27 by incubating for 2h at 37° C., Sigma) with changes every 2-3 days.

Mature cardiac organoids were incubated for 2 days with 0.5 mM mevalonate using the same protocols depicted in (FIG. 3A).

### Force Analysis of hCO in Heart-Dyno

The pole deflection was used to approximate the force of contraction as per (Mills et al., 2017a). A Leica DMi8 inverted high content Imager was used to capture a 10 s time-lapse of each hCO contracting in real time at 37° C. Custom batch processing files were written in Matlab R2013a (Mathworks) to convert the stacked TIFF files to AVI, track the pole movement (using vision.PointTracker), determine the contractile parameters, produce a force-time figure, and export the batch data to an Excel (Microsoft) spreadsheet.

Whole-Mount Immunostaining hCOs were fixed for 60 min with 1% paraformaldehyde (Sigma) at room temperature and washed 3× with PBS, after which they were incubated with primary antibodies (see Star Methods Table) in Blocking Buffer, 5% FBS and 0.2% Triton-X-100 (Sigma) in PBS overnight at 4° C. Cells were then washed in Blocking Buffer 2× for 2 h and subsequently incubated with secondary antibodies (see Star Methods Table) and Hoechst33342 (1:1000) overnight at 4° C. They were washed in Blocking Buffer 2× for 2 h and imaged in situ or mounted on microscope slides using Fluoromount-G (Southern Biotech).

### hCO Immunostaining Analysis

For screening hCOs were imaged using a Leica DMi8 high content imaging microscope for in situ imaging. Custom batch processing files were written in Matlab R2013a (Mathworks) to remove the background, calculate the image intensity, and export the batch data to an Excel (Microsoft) spreadsheet.

For more detailed images an Olympus IX81 confocal microscope was used for mounted hCO imaging. For cell cycle analysis experiments 3 random fields of view were imaged and manually quantified for proliferation. These were added together to calculate the percentage of hPSC-CM proliferation in each hCO.

### Cardiomyocyte Counting Analysis

Cells were dissociated by washing in perfusion buffer at 37° C. (130 mM NaCl, 1 mM MgCl2, 5 mM KCl, 0.5 mM NaH2PO4, 10 mM HEPES, 10 mM Taurine, 10 mM glucose, 10 μM 2,3-butanedione monoxime, pH 7.4). hCO were then incubated in EDTA buffer at 37° C. for 5 min (130 mM NaCl, 5 mM KCl, 0.5 mM NaH2PO4, 10 mM HEPES, 10 mM Taurine, 10 mM glucose, 5 mM EDTA, 10 μM 2,3-butanedione monoxime, pH 7.4). hCO were washed in perfusion buffer and then incubated in perfusion buffer plus 1 mg/ml collagenase B (Roche) for 15 min at 37° C. on a shaker at 500 rpm. Equivolume 0.25% trypsin-EDTA (ThermoFisher) was then added and the hCOs were incubated for 10 min at 37° C. on a shaker at 500 rpm. Perfusion buffer with 5% FBS was then added and the single cells pelleted by centrifuging at 1000×g for 3 min. The cells were then resuspended in 1% para-formaldehyde and incubated for 5 min at room temperature. hCO were then centrifuged at 1000×g for 3 min, paraformaldehyde removed, suspended in PBS and counted using a haemocytometer.

### RNA Sequencing

For each experiment 15-20 hCOs were pooled, snap frozen and RNA extracted using Trizol (ThermoFisher Scientific), treated with DNAse (Qiagen) and purified using RNeasy Minielute Cleanup Kit (Qiagen). The quality of the RNA was assessed by Fragment Analyzer (Advances Analytical Technologies) and samples with RNA quality number >8.9 were used for library preparation. RNA samples were processed with Illumina TruSeq Stranded mRNA Library prep kit selecting for poly(A) tailed RNA following the manufacturer's recommendations. Libraries were quantified with Qubit HS (ThermoFisher) and Fragment Analyzer (Advances Analytical Technologies) adjusted to the appropriate concentration for sequencing. Indexed libraries were pooled and sequenced at a final concentration of 1.8 pmol/L on an Illumina NextSeq 500 high-output run using paired-end chemistry with 75 bp read length.

### Proteomics and Data Processing

Single hCO were washed 2× in PBS and snap frozen and stored at −80° C. Tissues were lysed in by tip-probe sonication in 1% SDS containing 100 mM Tris pH 8.0, 10 mM tris(2-carboxyethyl)phosphine, 40 mM 2-chloroacetamide and heated to 95° C. for 5 min. Proteins were purified using a modified Single-Pot Solid-Phase-enhanced Sample Preparation (SP3) strategy (Hughes et al., 2014). Briefly, Proteins were bound to Sera-Mag carboxylate coated paramagnetic beads in 50% acetonitrile containing 0.8% formic acid (v/v) (ThermoFisher Scientific). The beads were washed twice with 70% ethanol (v/v) and once with 100% acetonitrile. Proteins were digested on the beads in 100 mM Tris pH 7.5 containing 10% 2,2,2-Trifluoroethanol overnight at 37° C. with 200 ng of sequencing grade LysC (Wako Chemicals) and trypsin (Sigma). Beads were removed and peptides acidified to 1% trifluoroacetic acid prior to purification by styrene divinyl benzene-reversed phase sulfonated solid phase extraction microcolumns. Peptides were spiked with iRT peptides (Biognosys) and analysed on an Easy-nLC1200 coupled to a Q-Exactive HF in positive polarity mode. Peptides were separated using an in-house packed 75 μm×50 cm pulled column (1.9 μm particle size, C18AQ; Dr Maisch) with a gradient of 2-35% acetonitrile containing 0.1% FA over 120 min at 300 nL/min at 60° C. The instrument was operated in data-independent acquisition (DIA) mode essentially as described previously (Bruderer et al., 2017). Briefly, an MS1 scan was acquired from 350-1650 m/z (120,000 resolution, 3e6 AGC, 50 ms injection time) followed by 20 MS/MS variable sized isolation windows with HCD (30,000 resolution, 3e6 AGC, 27 NCE). A spectral library was created by fractionating a pooled mix of peptides from 10 separate hCO on an inhouse packed 320 μm×25 cm column (3 μm particle size, BEH; Waters) with a gradient of 2-40% acetonitrile containing 10 mM ammonium formate over 60 min at 6 μL/min using an Agilent 1260 HPLC. A total of 12 concatenated fractions were analysed using the identical LC-MS/MS conditions above except the instrument was operated in data-dependent acquisition (DDA) mode. Briefly, an MS1 scan was acquired from 350-1650 m/z (60,000 resolution, 3e6 AGC, 50 ms injection time) followed by 20 MS/MS with HCD (1.4 m/z isolation, 15,000 resolution, 1e5 AGC, 27 NCE). DDA data were processed with Andromeda in MaxQuant v1.5.8.3 (Cox and Mann, 2008) against the human UniProt database (January 2016) using all default settings with peptide spectral matches and protein false discovery rate (FDR) set to 1%. DIA data were processed with Spectronaut v11 (Bruderer et al., 2015) using all default settings with precursor and protein FDR set to 1% and quantification performed at MS2.

### RNA-Seq Bioinformatics

The sequencing data was demultiplexed using Illumina bc12fastq2-v2.17. The quality of the reads was assessed thanks to FastQC (https://www.bioinformatics.babraham.ac.uk/projects/fastqc/). The reads were then processed and mapped to the human genome hg38 using the Bcbio-nextgen framework version 1.0.3 (https://github.com/chapmanb/bcbio-nextgen). The aligner used was HISAT2 2.0.5. For sample quality assessment, raw counts were normalised with DESeq2 regularized logarithm function. A principal component analysis (PCA) was performed using R v 3.3.1(Team, 2015) and led to the detection of a batch effect among samples which was corrected using the removeBatchEffect function from Limma v3.30.13. The identification of the differentially expressed genes between treated and non-treated samples was performed using DESeq2 v1.14.1 (Love et al., 2014) in R v 3.3.1 (Team, 2015) using the design ˜Batch+Compound to account for the batch effect directly. A FDR of 0.1 was defined to call for differentially expressed genes. The mechanistic networks in were generated using IPA upstream analysis (QIAGEN Inc., https://www.qiagenbioinformatics.com/products/ingenuity-pathway-analysis). Gene Ontology (GO) annotations enrichment analyses were performed with GOSeq v1.28.0 (Young et al., 2010), were generated in R using ggplot2 (http://ggplot2.tidyverse.org/index.html) and package GOSemSim v2.2.0 (Yu et al., 2010) for term similarities.

### Proteomic Bioinformatics

Proteomic data were Log 2 transformed and median normalised. Removal of unwanted variation method (RUV) was applied to remove any batch effects (Gagnon-Bartsch et al., 2013). Differential abundance was determined with two sample t-tests with FDR-based multiple hypothesis testing correction. GO analysis was performed with DAVID v6.8 and gene ontology terms from BP_FAT (Huang da et al., 2009) and heat-maps and hierarchical clustering was performed using GENE-E (Broad Institute).

### 2D Cell Culture

Day 15 dissociated differentiation cultures were plated at 100,000 cells/cm2 in 0.1% gelatin coated 96-well tissue culture plates in CTRL medium. For 3 day incubation experiments, after 2 days of culture in CTRL medium, cells were then treated with 10 μM simvastatin in maturation medium for a further 3 days. For 1 day incubation experiments, after 1 day of culture in CTRL medium, cells were treated with 10 μM simvastatin, 0.5 M (±)-Mevalonic acid 5-phosphate lithium salt hydrate, 20 μM geranylgeranyl pyrophosphate, 20 μM farnesyl pyrophosphate and/or 20 μM squalene in maturation medium for a further day.

### 2D Immunostaining

hCOs were fixed for 10 min with 1% paraformaldehyde (Sigma) at room temperature and washed 3× with PBS, after which they were incubated with primary antibodies (see Star Methods Table) in Blocking Buffer, 5% FBS and 0.2% Triton-X-100 (Sigma) in PBS for 1-2 hours at room temperature. Cells were then washed in Blocking Buffer 2× for 3 min and subsequently incubated with secondary antibodies (see Star Methods Table) and Hoechst33342 (1:1000) for 1 hour at room temperature. They were washed in Blocking Buffer 2× and imaged.

### 2D Immunostaining Analysis

For screening hCOs were imaged using a Leica DMi8 high content imaging microscope for in situ imaging. Custom batch processing files were written in Matlab R2013a (Mathworks) to analyse the number of cardiomyocytes, identify the nuclei of proliferating (Ki-67+) cardiomyocytes and determine the average size of the cardiomyocytes, and export the batch data to an Excel (Microsoft) spreadsheet.

### Neonatal Simvastatin Treatment

Postnatal day 1 mice (P1) were given daily s.c. injections of vehicle (DMSO, 1 uL/g) or simvastatin (10 mg/kg/day) until P15. Animals were BrdU pulsed with 100 mg/kg i.p. injections at day P1, P3, P5, P7, P9, P11, P13 and P15. Hearts were collected at P25 for analysis.

### Heart Sectioning and Staining

For BrdU and WGA staining, mice were sacrificed by cervical dislocation and hearts collected, washed in PBS and fixed in 4% paraformaldehyde overnight. Each heart was washed in PBS, halved with a single transverse cut, dehydrated and embedded in paraffin wax. 6 μm sections were mounted on SuperFrost Ultra Plus slides. Sections were then rehydrated and blocked with 10% goat serum in PBS. Sections were stained with BrdU and MLC2v antibodies, relevant secondary antibodies (see Star Methods Table) and Hoechst (1:1000) to quantify proliferating cardiomyocytes. Sections were also stained with WGA (see Star Methods Table) to quantify the cross-sectional area of cardiomyocytes. The samples were then imaged using a Zeiss NLO 780 point scanning confocal microscope. For BrDU quantification 3 random regions were imaged at 2 different levels per heart. Random images were taken for WGA size quantification for each heart and 20 cardiomyocytes randomly assessed per heart. All sample preparations and analyses were performed with the experimentalist blinded to the conditions.

### Adult Mouse Intracardiac Injections of Compounds

8-week-old male mice were anesthetized with 4% isoflurane (Bayer) with 0.25 L/min oxygen and then maintained with 2% isoflurane. Once, anaesthetised, each mouse received a s.c. buprenorphine injection (0.05 mg/kg). The animals were intubated and ventilated (Minivent, Harvard Apparatus) (tidal volume=250 respiration rate=133 strokes/min). To access the heart, a lateral thoracotomy was performed at the 4th intercostal space. A Hamilton syringe with a 30-gauge needle was used to inject ˜20 [symbol] l of small molecule solution (final concentration 10 mg/kg) into the myocardium. All compounds were emulsified in Kolliphor/PBS solution (20% Kolliphor® HS 15 (w/v) in PBS, pH 7.4) at a concentration of 20 mg/mL. The chest wall was closed using 4-0 silk suture, the skin closed with 6-0 prolene suture and the mouse removed from anaesthesia. Following anaesthesia, mice were supplied with a s.c. injection of buprenorphine (0.05 mg/kg) and carprofen (10 mg/kg) and allowed to recover. Starting from the day of the surgery, mice received daily 5-bromo-2′-deoxyuridine (BrdU) injections (100 mg/kg, i.p.). Simvastatin (20 mg/kg, in Kolliphor/PBS solution, s.c.) or control (Kolliphor/PBS solution, s.c.) was also injected daily.

Fix-Dissociation and Analysis of Cardiomyocytes from Adult Hearts

3 days post-surgery, mice were sacrificed, the hearts removed and washed in PBS and then fixed overnight at room temperature with 1% paraformaldehyde/PBS solution. The hearts were then washed 3 times in PBS. The atria were removed, and the ventricles were diced with scissors to approximately 1 mm3. Diced ventricular tissue was then placed in 1 mL collagenase B (Roche) solution (2 mg/ml collagenase B in PBS with 0.02% NaN3) and oscillated (1000 rpm) at 37 [symbol] C. Every 12 hours the diced tissue was allowed to settle and the supernatant containing cells was collected and stored in FBS (containing 0.02% NaN3) at 4 [symbol] C. A further 1 mL of collagenase solution was added and collections continued 12 hourly until all the heart tissue was cellularized.

Cardiomyocytes were the counted, cytospun onto glass slides and stained with BrdU and MLC2v antibodies, relevant secondary antibodies (see Star Methods Table) and Hoechst (1:1000) to quantify proliferating cardiomyocytes. The AlexaFluor 647 was used to stain BrDU to avoid false positive associated with auto-fluorescence inherent in adult cardiomyocytes in blue-green-yellow channels. To quantify proliferation, 5,000 cardiomyocytes per heart were imaged using a Nikon Spinning Disc confocal microscope using a 20× objective.

### Quantifying Prenylated Proteins

Targeted data extraction to quantify prenylated peptides was performed manually in the Skyline Environment (MacLean et al., 2010) on Andremoda/MaxQuant search results which included the variable modification of farnesylation (cysteine; C15H24; 204.1878) and geranylation (cysteine; C201-132; 272.2504) with neutral loss. Only peptides with an Andromeda score >100 and a localization probability >0.75% were included in the analysis.

### Quantification and Statistical Analysis

All key hCO experiments were performed with multiple hCO per condition in multiple experiments to ensure reproducibility. For screening or experiments where multiple groups were analyzed, all groups were present in each experiment including controls to ensure that results were not an artefact of comparing conditions over different experiments. Personnel performing the animal experiments and analyses were blinded to the conditions or treatments.

Data is presented as mean±S.E.M. unless otherwise noted. Statistics were analysed using Microsoft Excel (Microsoft) or GraphPAD Prism 6 (Graphpad Software Inc). Sample numbers, experimental repeats, statistical analyses and p-values are reported in each figure legend. Data is presented as n=replicates, from x number of independent experiments.

### Data and Software Availability

RNA-seq data has been deposited in GSE111853 and will be made publicly available following acceptance of the manuscript. All proteomics raw data, MaxQuant and Spectronaut data have been deposited in PRIDE under PXD009133 and will be publicly available following acceptance of the manuscript. All Matlab m-files will be provided upon request.

### Results

### Functional Screening in hCO Identifies Putative Pro-Proliferation Compounds

A drug development pipeline was established to move potential pro-regenerative small molecules progressively through screens with increasing complexity and maturation status, followed by dissection of underlying mechanisms driving proliferation of cardiomyocytes (FIG. 1). Compounds were screened in 2D hPSC-CM, then in immature proliferative hCO, followed by validation in mature, cell cycle-arrested hCO (Mills et al., 2017a). Using this approach compounds were assessed for both induction of proliferation and functional side-effects. Furthermore, using RNA-sequencing and proteomic profiling, we defined underlying mechanisms of action (FIG. 1).

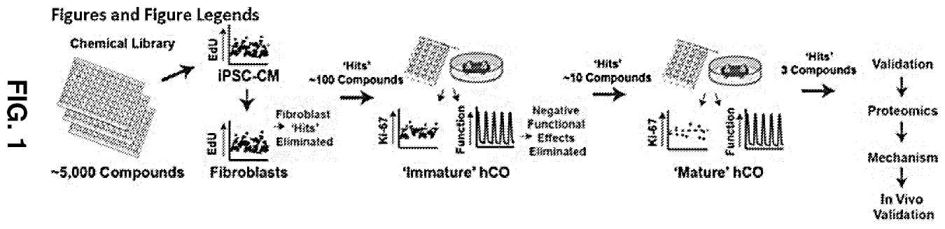

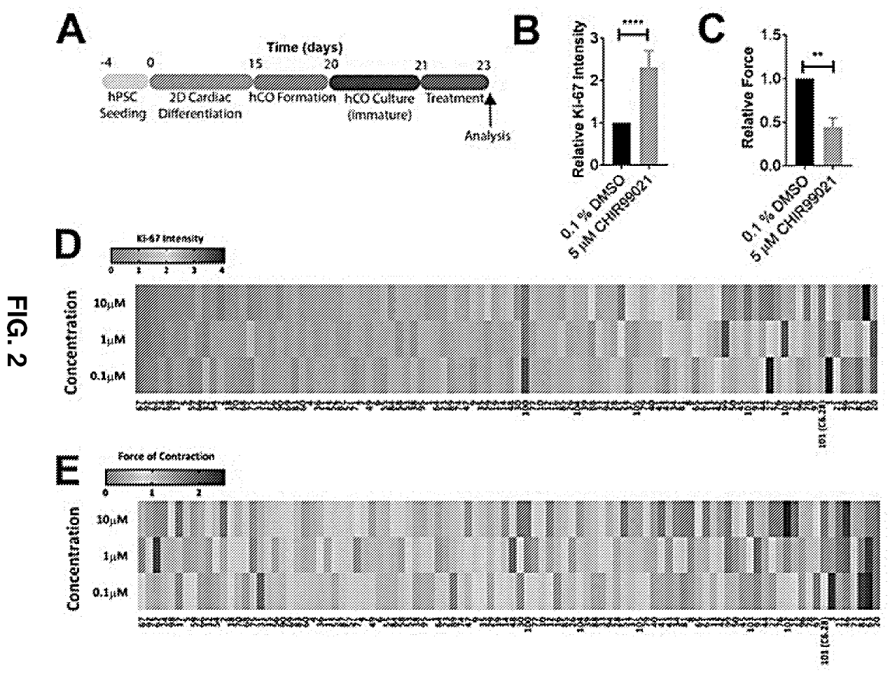

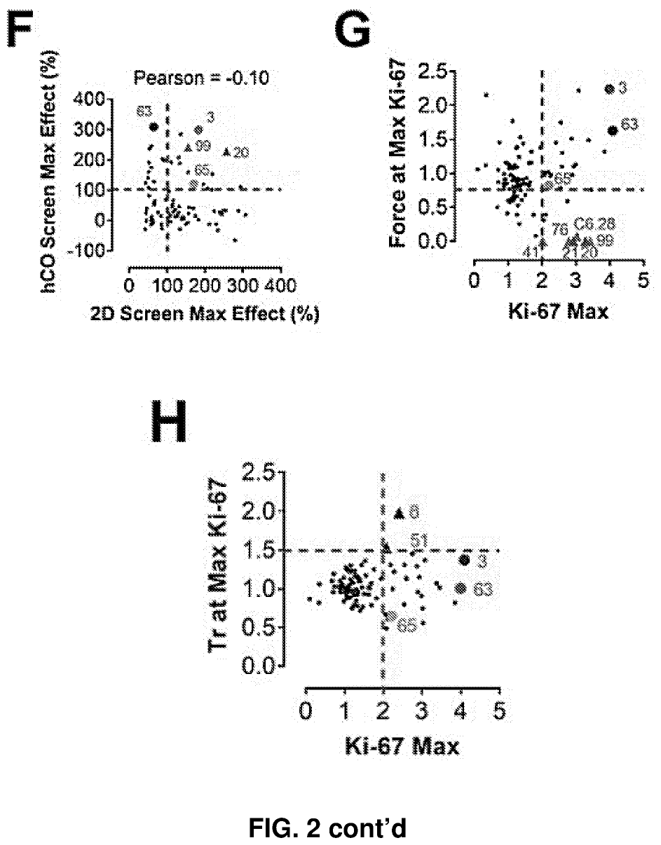

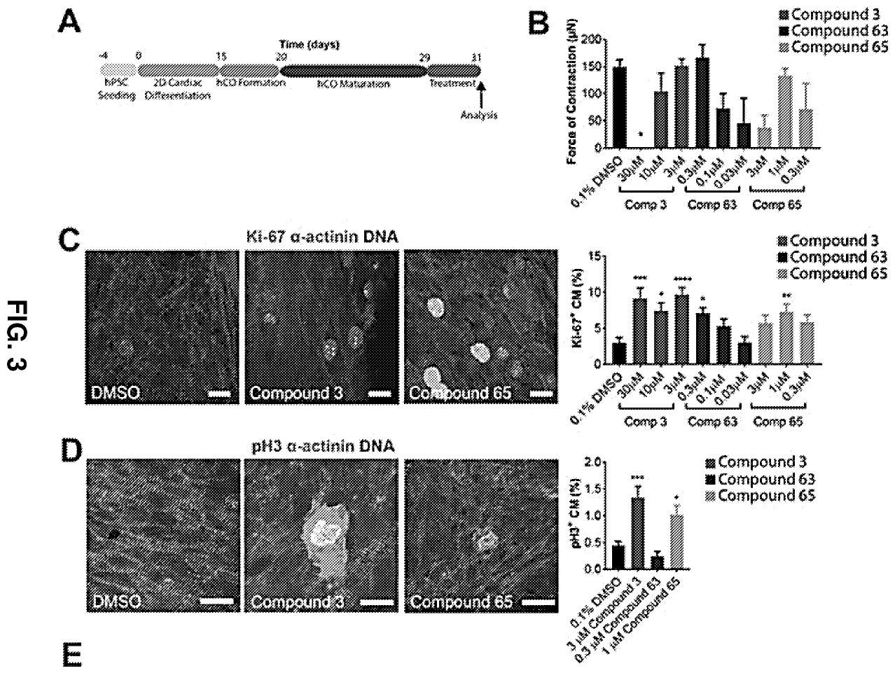

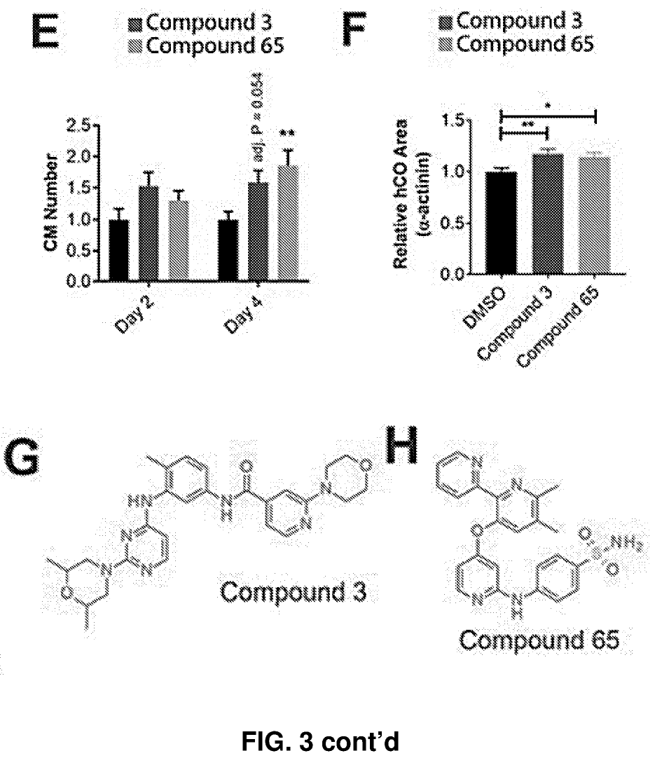

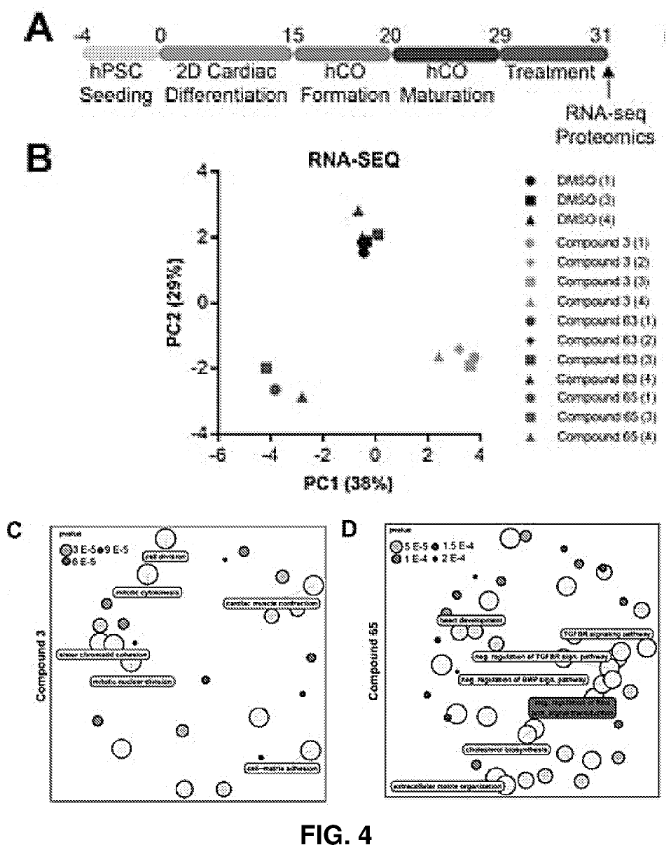

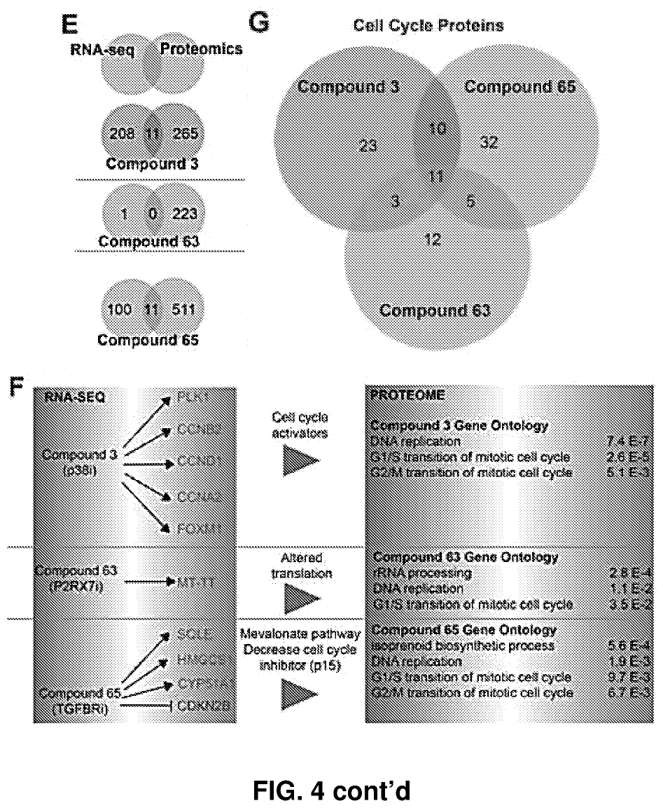

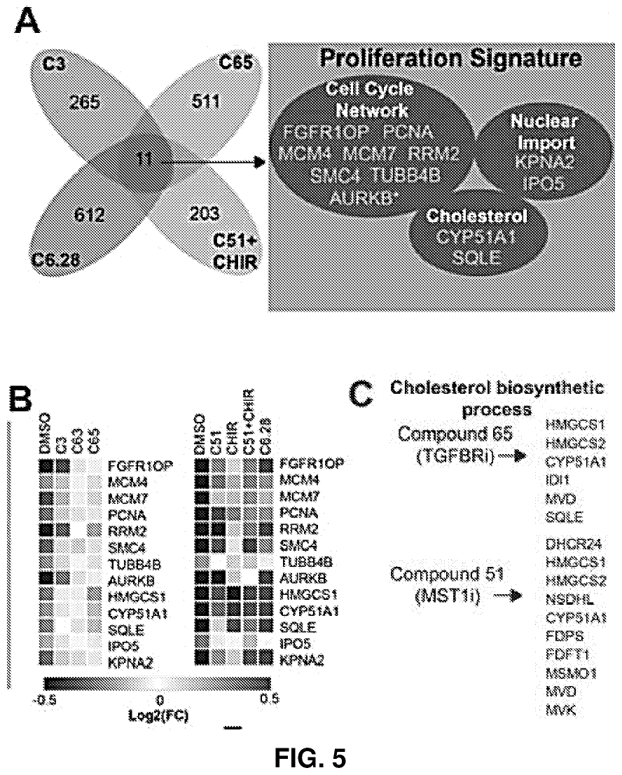

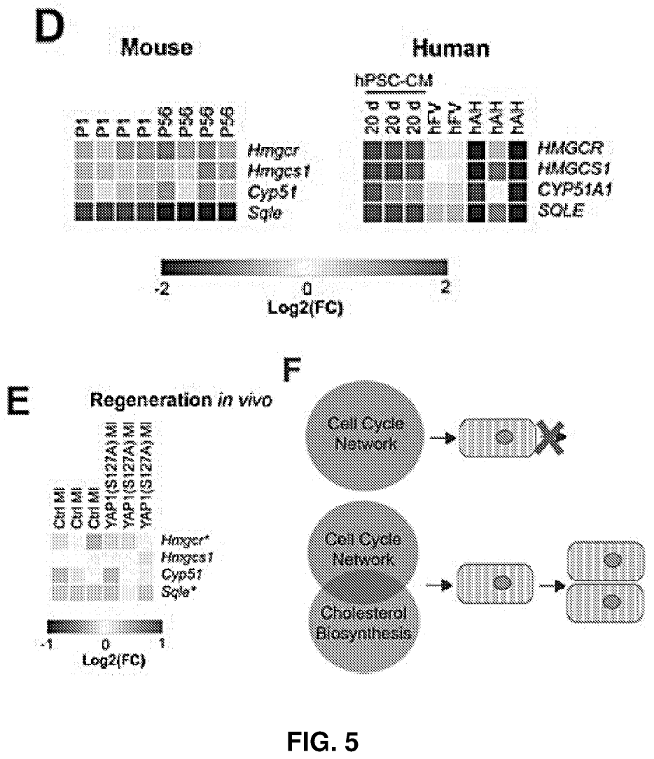

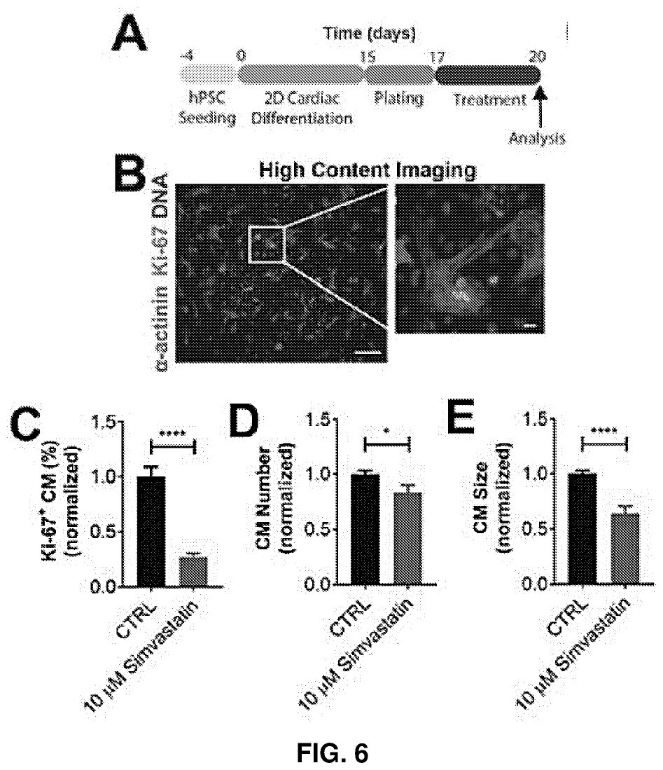

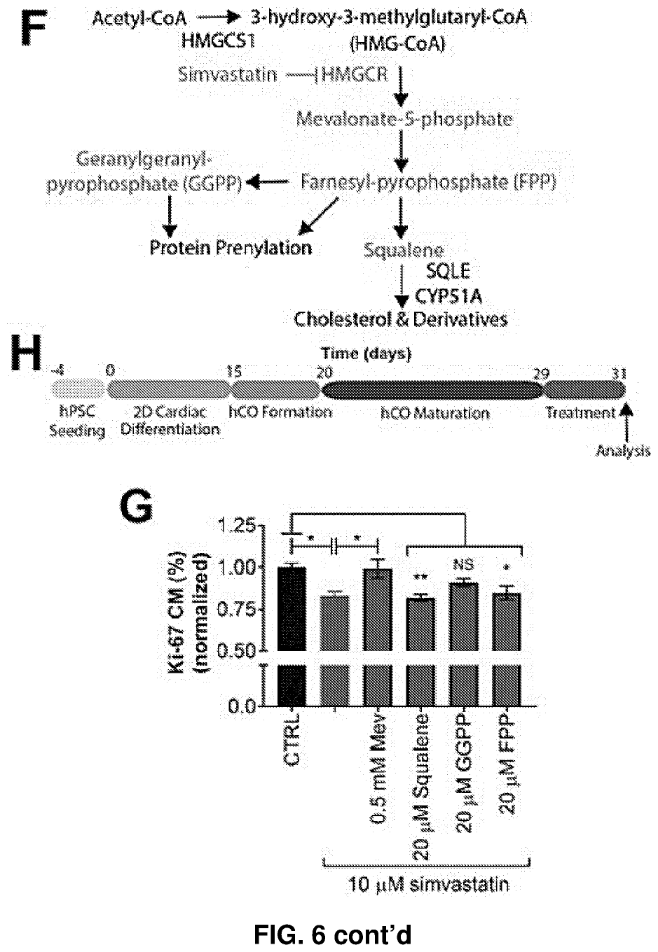

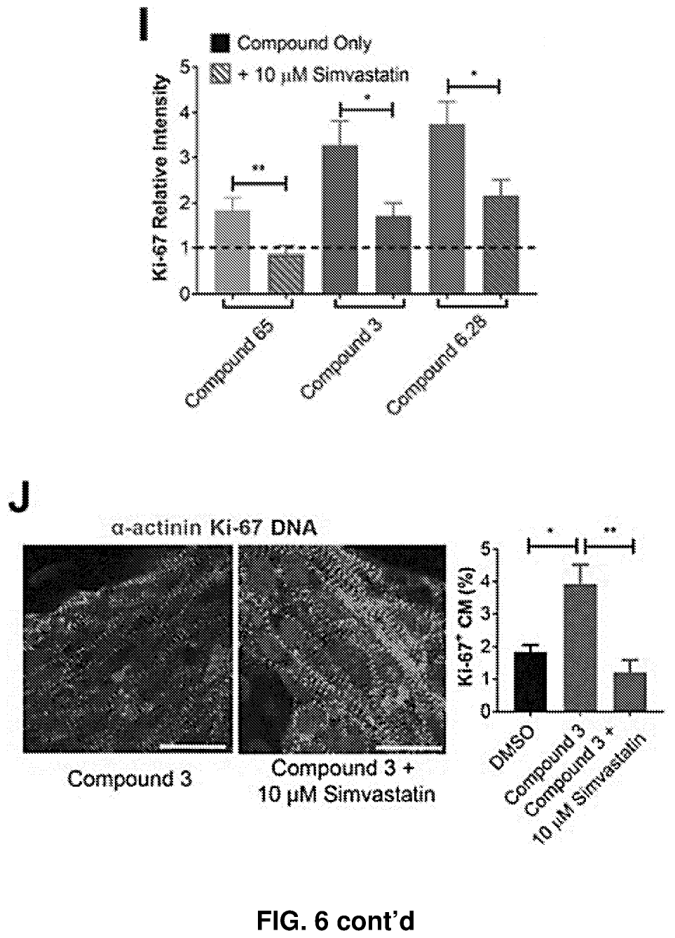

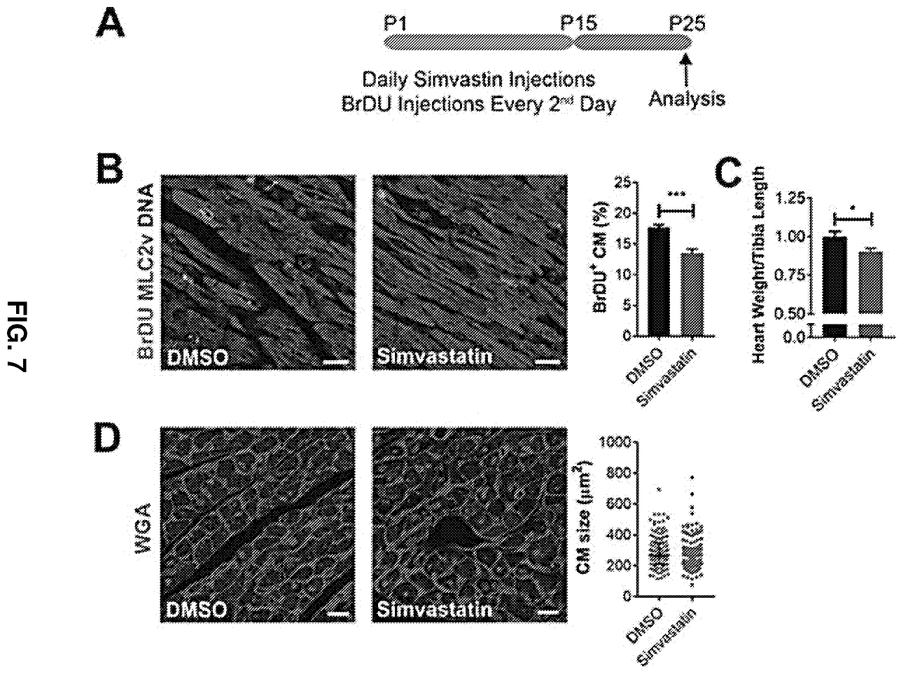

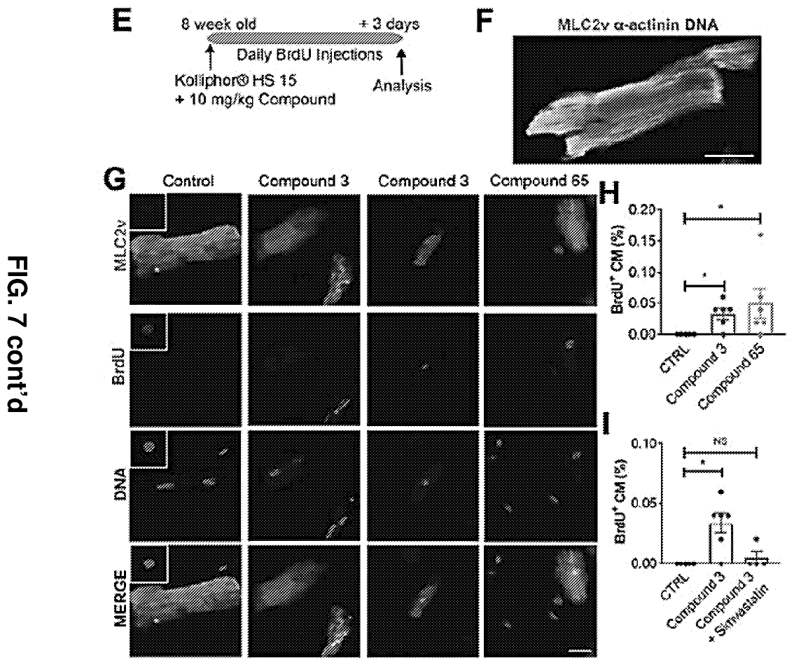

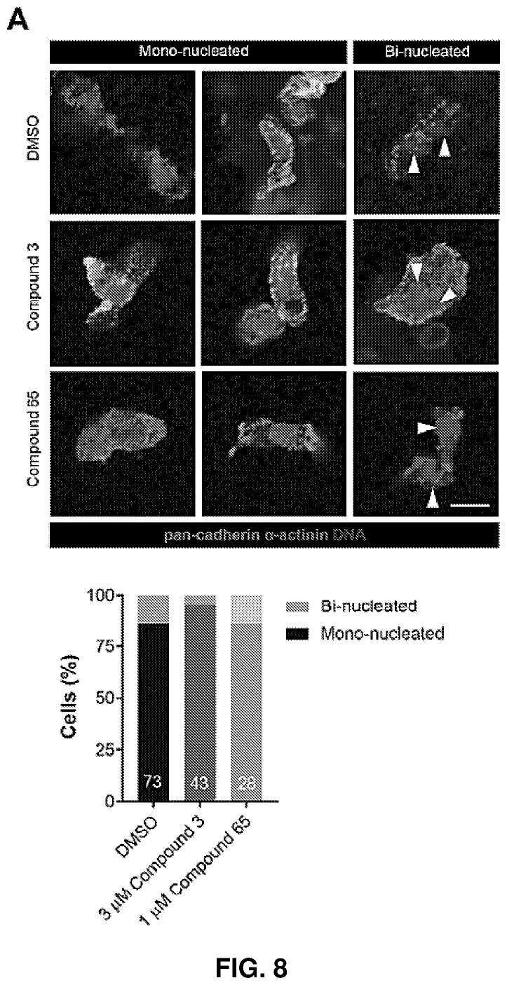

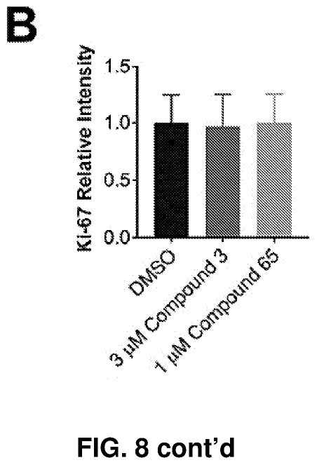

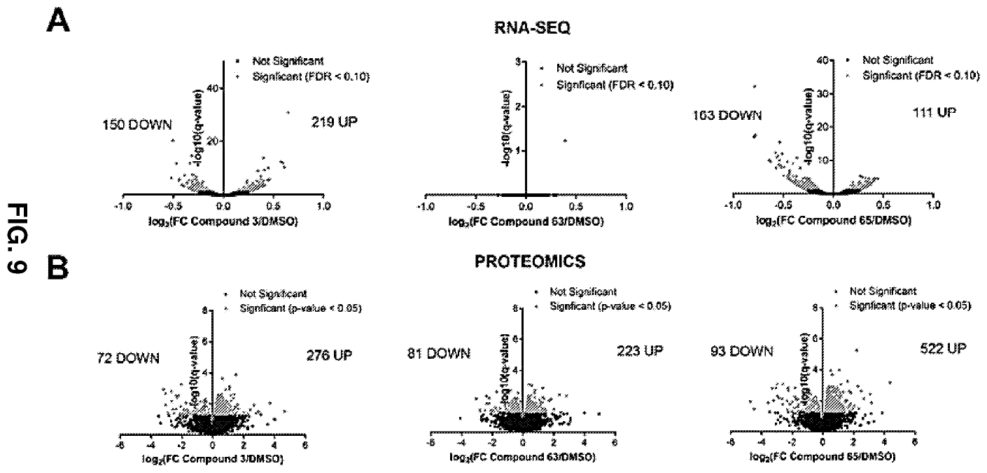

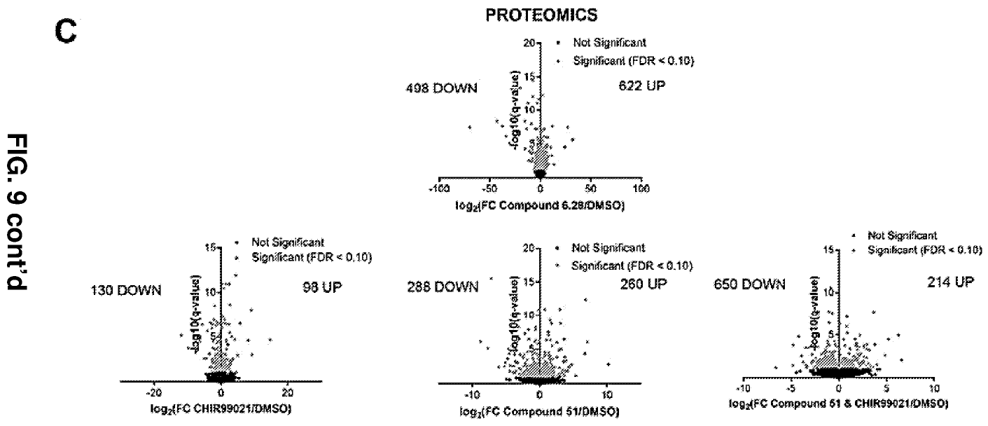

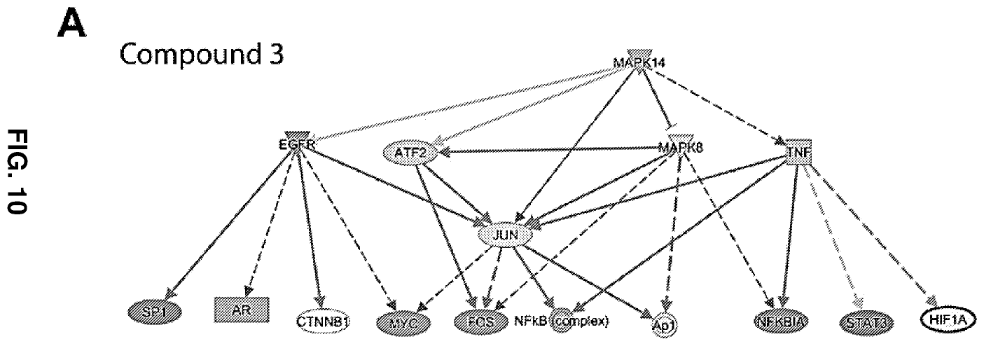

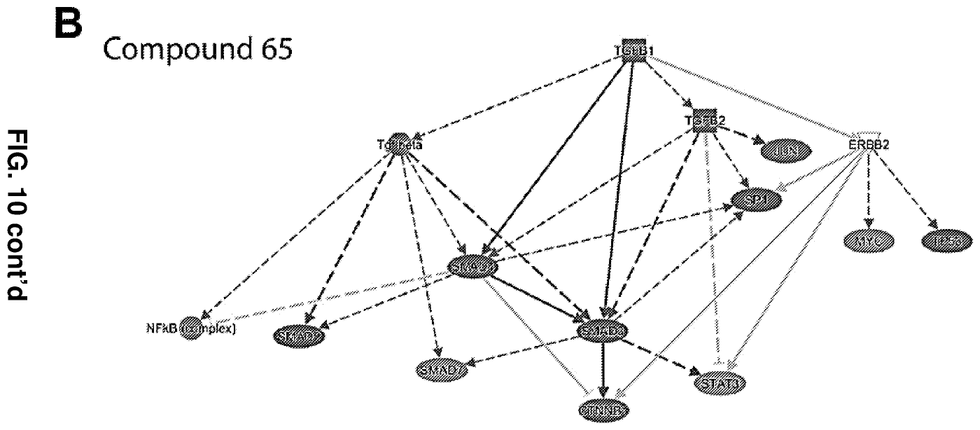

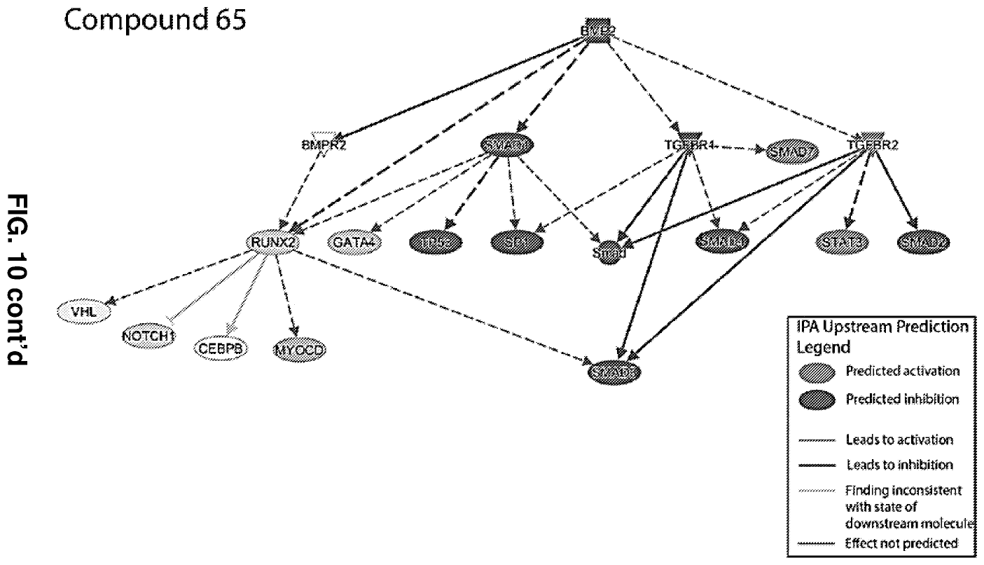

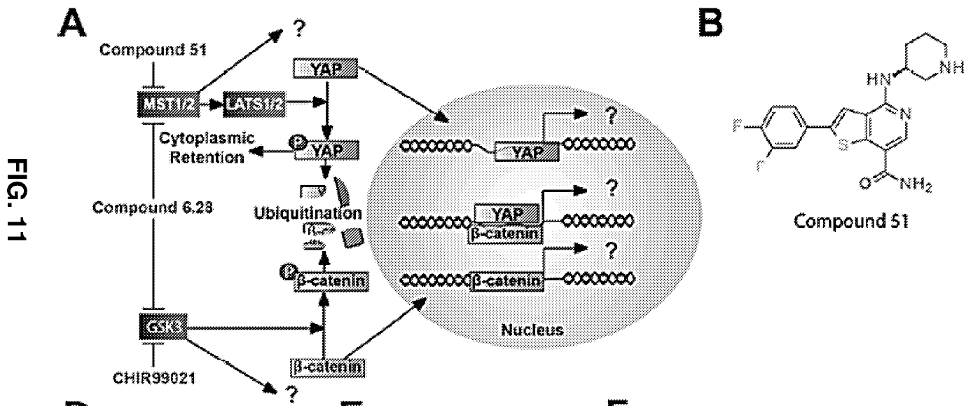

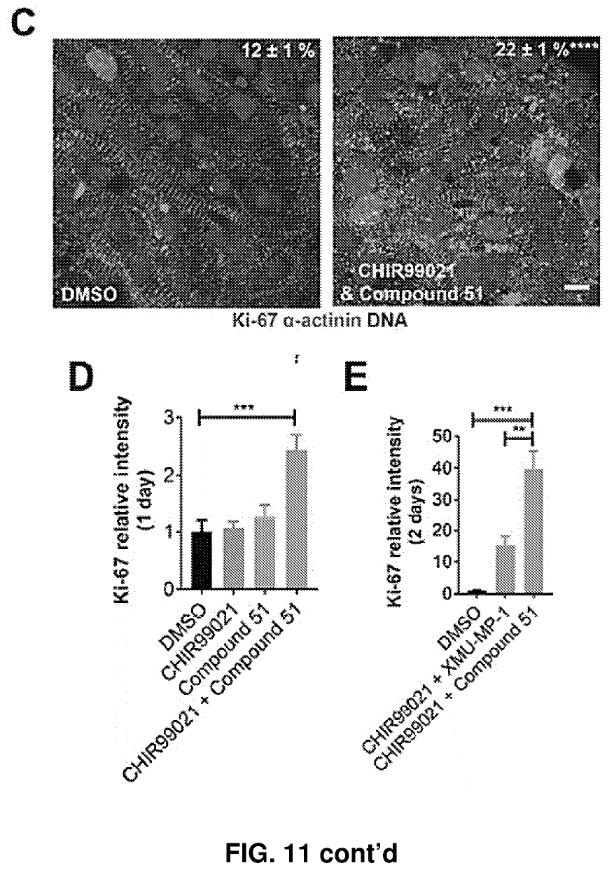

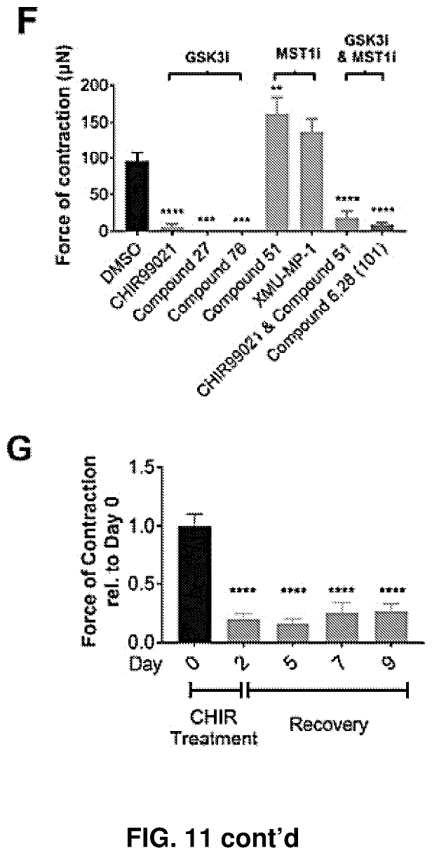

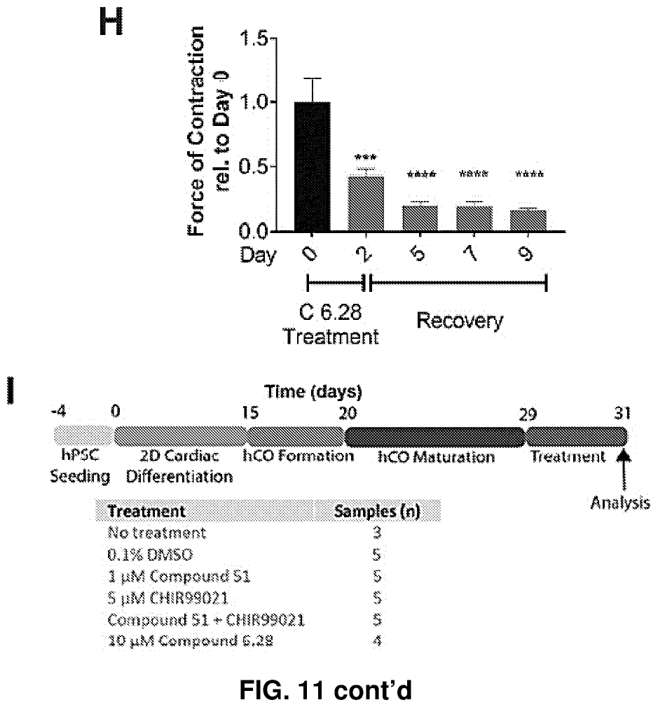

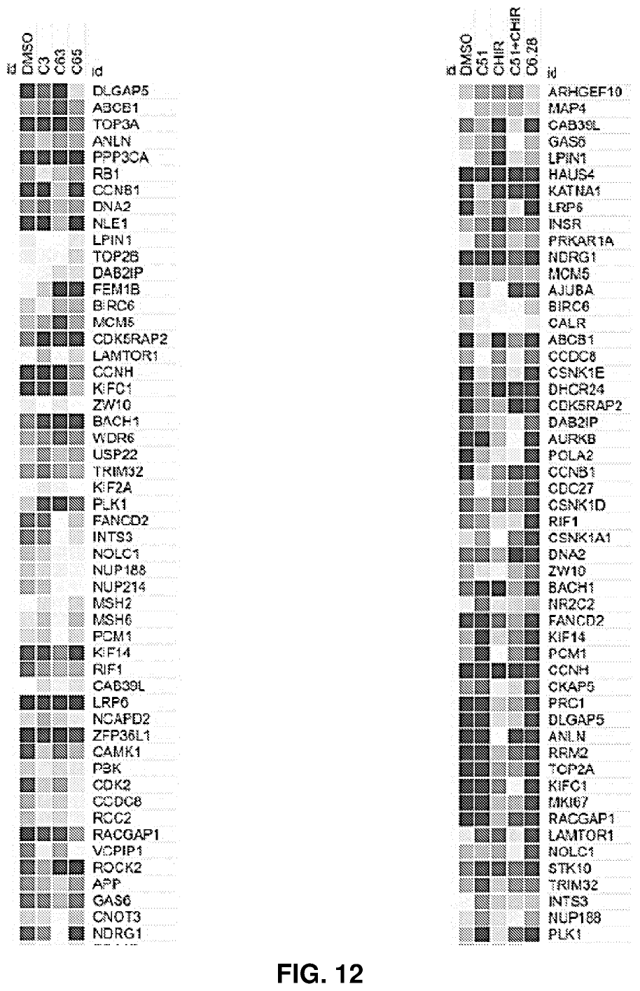

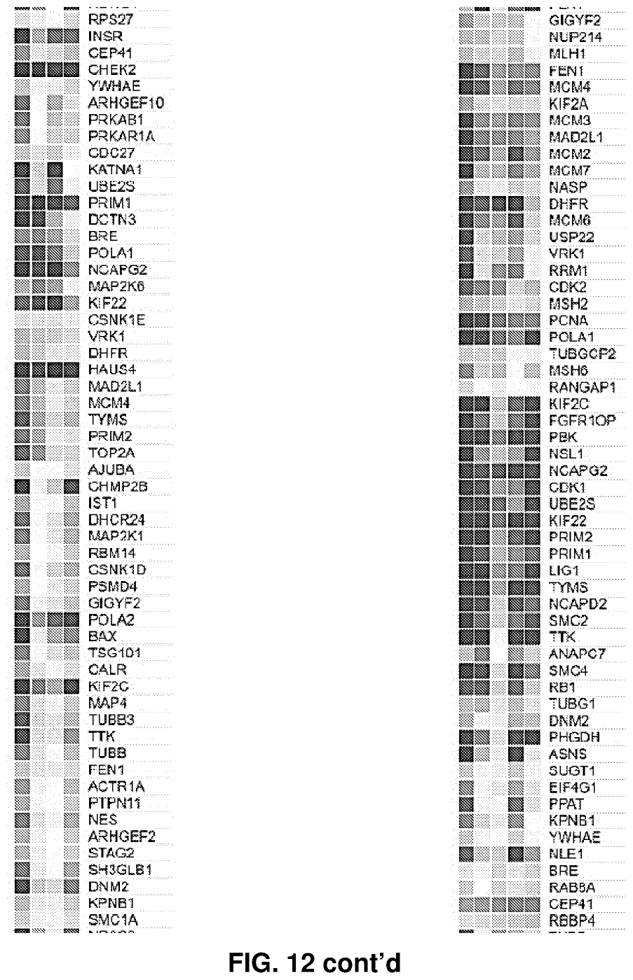

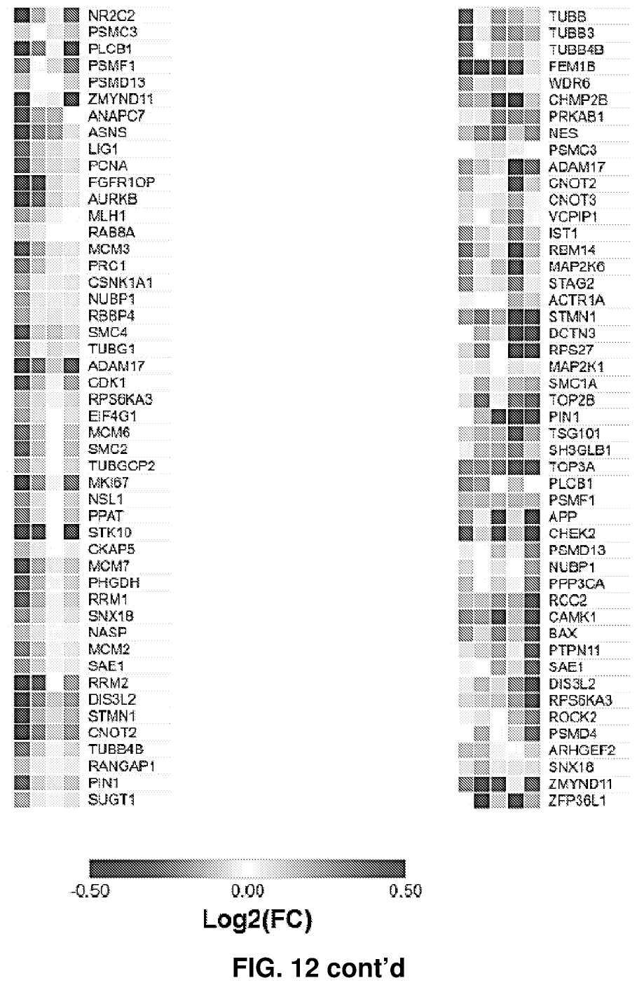

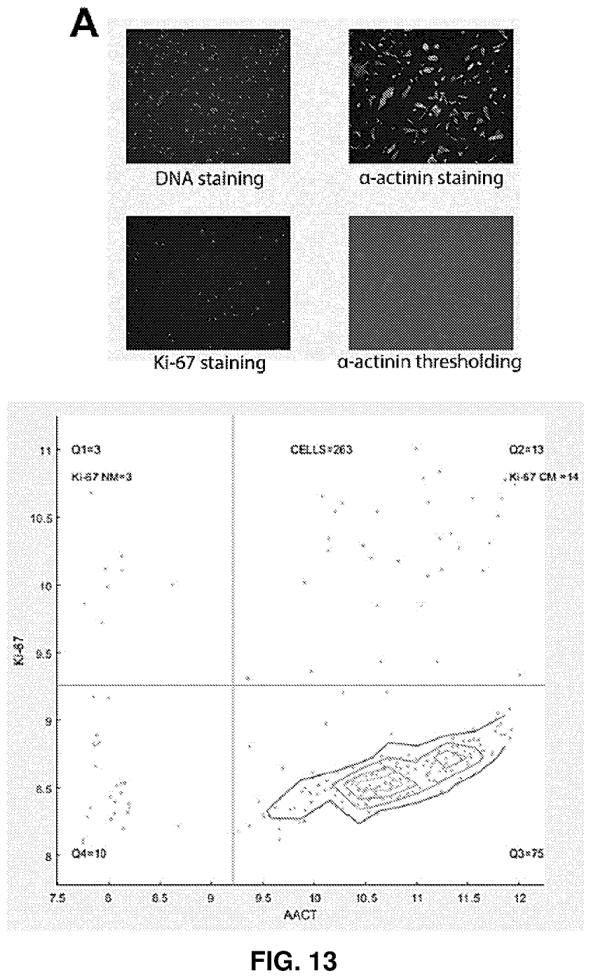

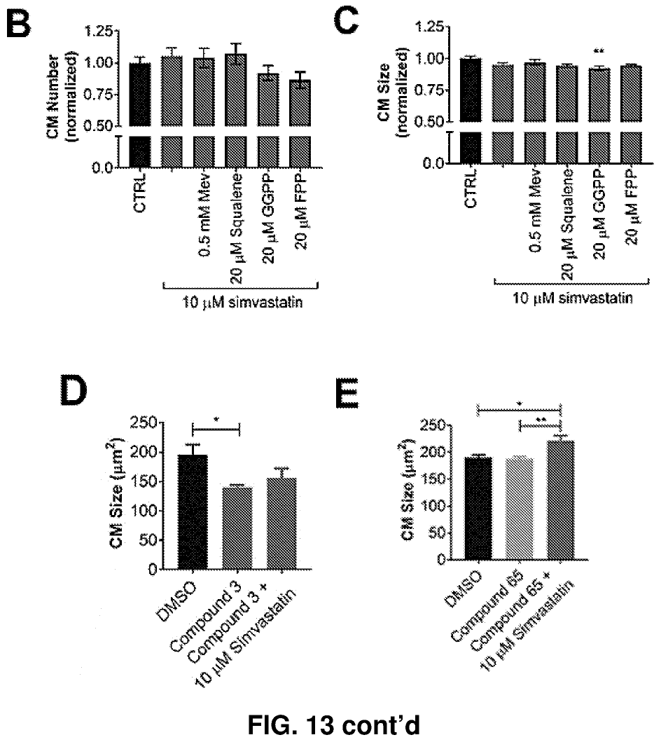

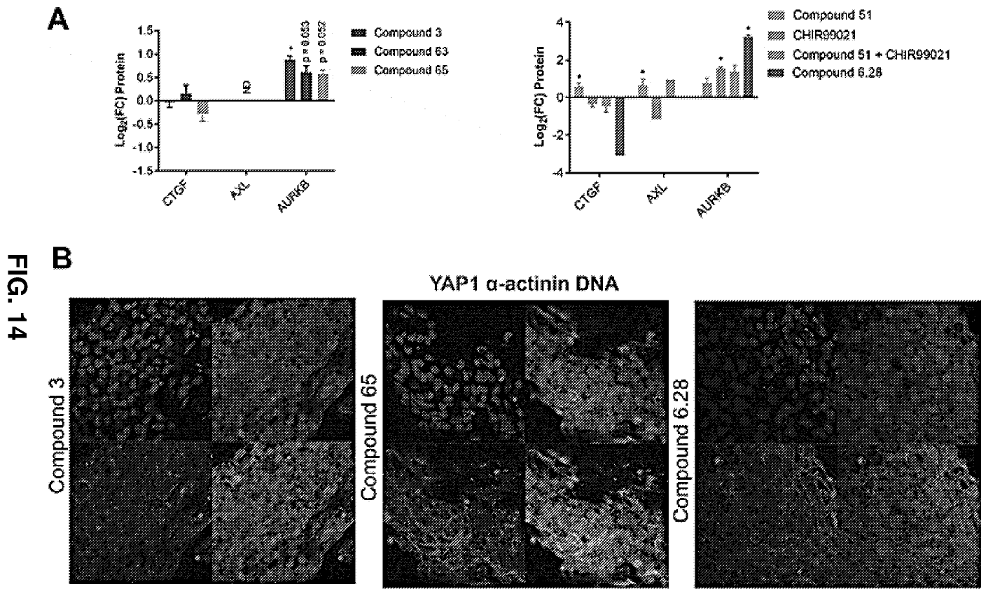

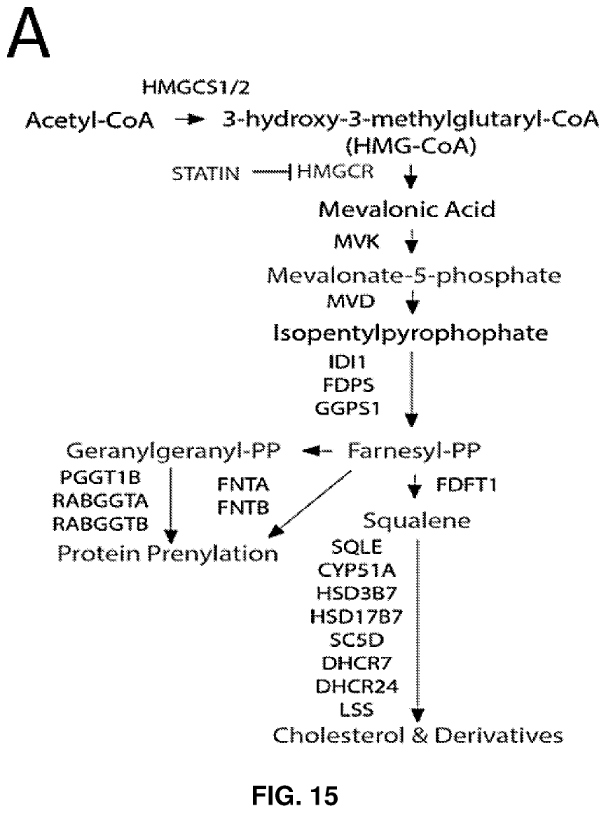

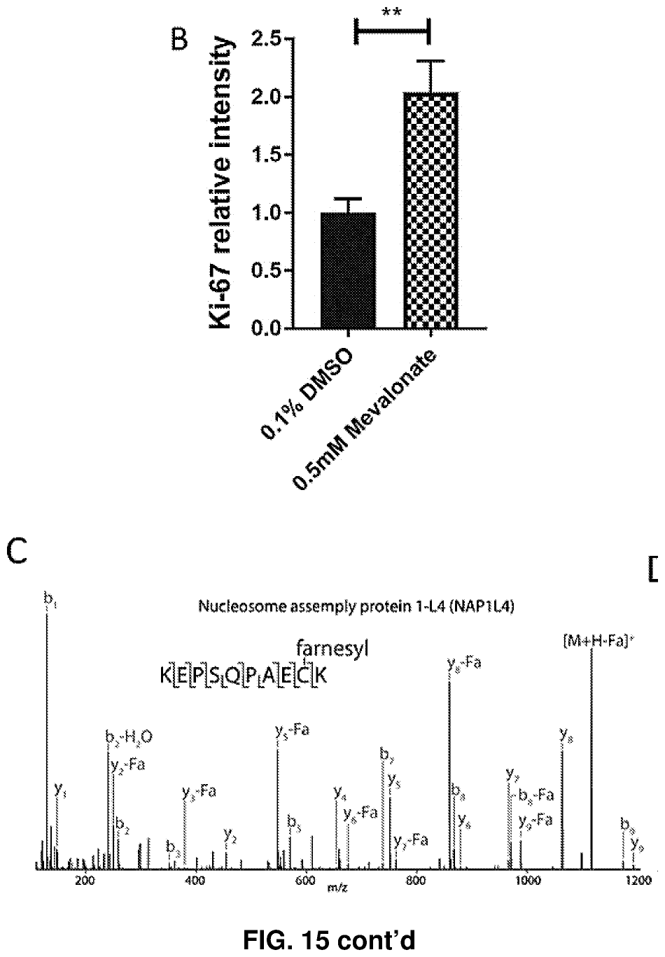

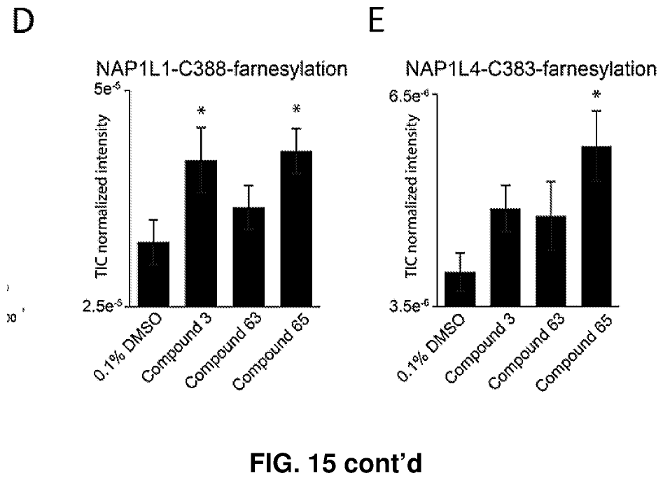

The compound library included 5,000 biologically annotated pre-clinical, clinical, and tool compounds. They were selected based on a combination of criteria including balancing the number of external/internal compounds, diversity of annotated targets to cover greater than 1500 biological targets, and known targets associated with cell proliferation. Initial screens were performed in a 2D high-content primary screen, measuring DNA synthesis with 5-ethynyl-2′-deoxyuridine (EdU) over 2 days and the hits were assayed in a counter-screen of cardiac fibroblasts to select compounds that preferentially induced proliferation in hPSC-CMs (data not shown). Subsequently, 105 potential hit compounds were screened over a 3 log-scale concentration range (0.1, 1, and 10 μM, 1,000 hCO) in immature hCOs, which have a robust proliferative response to mitogenic stimuli (Mills et al., 2017a; Voges et al., 2017) (FIG. 2A). Both negative vehicle (DMSO) and positive (CHIR99021) controls were included on each plate in each experiment (FIG. 2B,C).

Screening identified several small molecular weight molecules that were capable of inducing proliferation (FIG. 2D) and the top 9 hits in the hCO also induced >50% increase in proliferation in the 2D assay (Table 1). Intriguingly, many compounds that induced proliferation in 2D failed to induce proliferation even in the immature hCOs (FIG. 2F, Table 1).

A switch to proliferation could have consequences on the contractile apparatus or calcium signalling, which may negatively impact function. Of the compounds that induced proliferation in the immature hCO, many decreased force of contraction (FIG. 2E,G). Of the 8 compounds that activated proliferation and reduced force to less than 10%, 5 of them inhibited GSK3 (green triangles) and 2 of them activated adenosine receptor 2A (purple triangles). Additionally, other pro-proliferative compounds (compounds 8 and 51) prolonged the 50% relaxation time (FIG. 2H), which is indicative of an increased risk of arrhythmogenesis (Mills et al., 2017a).

As heart regeneration occurs in the early mammalian heart in both rodents (Porrello et al., 2011; Porrello et al., 2013) and pigs (Ye et al., 2018; Zhu et al., 2018) without adversely affecting cardiac contractility, it should be possible to find compounds that do not impact function whilst activating a pro-proliferative response. Importantly, our screen identified compounds that induced proliferation without affecting contractile parameters (FIG. 2G,H). These compounds included inhibitors of three distinct pathways including p38α/β, P2RX7, and TGFβR/BMPR (compounds 3, 63 and 65, respectively), thus indicating that it is the signalling pathways rather than activation of proliferation per se that may impact force.

Hits were next validated in mature hCOs using a targeted concentration range based on their active concentrations in the initial immature hCO screen (FIG. 3A). Proliferation markers were quantified using high magnification confocal imaging as we found flow cytometry to be inaccurate in hCO (data not shown). Compounds 3, 63 and 65 had no impact on contractile force (FIG. 3B) at concentrations of 3 μM, 0.3 μM and 1 μM, respectively. At these concentrations, compounds 3, 63 and 65 all led to increased Ki-67+ cardiomyocytes in the hCOs (FIG. 3C). There was no further increase in proliferation above these concentrations for compounds 3 and 65, likely due to toxic effects at the highest doses (FIG. 3C). Interestingly, only compounds 3 and 65 promoted cell cycle progression and mitosis of cardiomyocytes in the hCO, marked by pH3+ cardiomyocytes with disassembled sarcomeres (FIG. 3D). Consistent with full proliferation to cytokinesis, there was also an increase in cardiomyocyte number following treatment with compound 3 or compound 65 (FIG. 3E) and an increase in hCO size (FIG. 3F) without an increase in bi-nucleation (FIG. 8A) or cardiomyocyte size (FIG. 13E). The effects of both compound 3 and compound 65 were reversible, with proliferation ceasing within 5 days of compound withdrawal (FIG. 8B). Therefore, compound 3 (FIG. 3G) and compound 65 (Goldberg et al., 2009) (FIG. 3H) are both capable of inducing bona fide cardiomyocyte proliferation in mature hCOs without adversely affecting contractile properties.

### Compounds Activating Proliferation Use Distinct Mechanisms of Cell Cycle Activation

In order to determine the biological processes driving the pro-proliferative effects of our hit compounds, we performed RNA-sequencing (RNA-seq) and single organoid quantitative proteomics (FIG. 4A). These analyses were performed on hCOs treated with compounds 3, 63 and 65 at concentrations of 3 μM, 0.3 μM and 1 μM, respectively. RNA-seq for hCO treated with compounds 3 and 65 clustered distinctly in principal component analysis (FIG. 4B) and regulated a total of 239 and 207 genes (FIG. 9A), respectively. Compound 63 significantly regulated only one gene (FIG. 9A) and clustered with DMSO controls in principal component analysis (FIG. 4B). Network analysis revealed that compound 3 significantly regulated a MAPK14 (p38α) network (FIG. 10A), which was expected based on the kinase inhibition profile for this compound and strong specificity in targeting p38α (Table 2). In support of our findings, p38α inhibition has been shown to regulate proliferation of adult rodent cardiomyocytes both in vitro (Engel et al., 2005) and in vivo (Engel et al., 2006).

Compound 65 regulated an extracellular matrix network and a cholesterol biosynthesis network based on RNA-seq data (FIG. 4D). Network analysis revealed compound 65 significantly regulated TGFβ receptor (TGFβR) and bone morphogenic protein receptor (BMPR) networks (FIG. 10B), which was also expected based on the kinase inhibition profile for this compound (Table 3). In support of a key role for TGFβR signalling in driving cell cycle activity, it has been recently demonstrated that the TGFβR inhibitor, SB431542, synergises with cyclin-dependent kinases to induce adult rodent cardiomyocyte proliferation in vivo (Kodo et al., 2016; Mohamed et al., 2018). However, SB431542 alone was not sufficient for robust pro-proliferative activity (Mohamed et al., 2018), suggesting that dual targeting of both TGFBR and BMPR may be required for the pro-proliferative actions of compound 65.

There was little overlap between RNA and protein regulation in response to compounds 3 and 65 (FIG. 4E). This is partly due to the low expression of some cell cycle proteins, and because of protein specific regulation of many factors in cardiac tissue (Mills et al., 2017a; Ulmer et al., 2018). A prime example of this is compound 63, which regulated only one transcript MT-TT (FIG. 4F), which is critical for protein translation. This resulted in upregulation of 223 proteins and down-regulation of 81 proteins, 11 of which were involved in rRNA processing (FIG. 4F). Compound 63 regulated a number of proteins involved in DNA replication and G1/S transition but not G2/M transition (FIG. 4F). This is consistent with compound 63 activating Ki-67 (FIG. 2F) but failing to promote progression to mitosis in mature hCO (FIG. 3D). This indicates that compound 63 only induces proliferation into G1, which only requires a limited number of proteins in comparison to full cell cycle progression, and this may also explain the lack of induction of a proliferation signature in the RNA-sequencing. Therefore, there is a distinction between transcriptional and proteomic regulation of cell cycle processes by different compounds.

Compound 3 upregulated the transcription of many cell cycle controllers including PLK1, CCNB2, CCND1, CCNA2 and FOXM1 in the RNA-seq data, resulting in activation of multiple cell cycle proteins including DNA replication, G1/S transition and G2/M transition in the proteome (FIG. 4F). Compound 65 inhibited the transcription of CDKN2B (p15) (FIG. 4F), which is the only CDK inhibitor expressed at a higher level in non-regenerating adult versus neonatal regenerating mouse hearts in vivo (Quaife-Ryan et al., 2017). Additionally, compound 65 activated multiple enzymes in the mevalonate pathway (also “isoprenoid biosynthetic process”) including SQLE, HMGCS1 and CYP51A1 (FIG. 4F). Together, compound 65 induced multiple cell cycle programs at the protein level including DNA replication, G1/S transition and G2/M transition (FIG. 4F).

Collectively, these findings suggest that each compound activated cell cycle through distinct mechanisms. In addition, each compound activated distinct cell cycle programs when comparing regulated proteins in the “cell cycle” ontology, and there were fewer cell cycle proteins activated by compound 63, which failed to induce full cell cycle progression (FIG. 4G, FIG. 12).

The “Proliferation Signature” is Associated with the Mevalonate Pathway

To determine whether there is a core “proliferation signature” that all pro-proliferative stimuli activate, we determined which proteins were similar in all pro-proliferative conditions. To expand our conditions for greater insight, we also included activation with compound 6.28, a dual inhibitor of GSK3 and MST1 (FIG. 11A) that is capable of inducing a robust proliferative response in mature hCO (Mills et al., 2017a). To also de-convolute the pro-proliferative effects of compound 6.28, which targets both GSK3 and MST1, we performed single organoid proteomics on hCO treated with CHIR99021, a GSK3 inhibitor (Mills et al., 2017a; Titmarsh et al., 2016), compound 51, an MST1 inhibitor (FIG. 11B), and, CHIR99021 and compound 51 in combination (dual GSK3 and MST1 inhibition). Importantly, similar to compound 6.28, co-administration of CHIR99021 and compound 51 synergistically induced cardiomyocyte proliferation in mature hCO (FIG. 11D). In confirmation of these findings, another MST1 inhibitor, XMU-MP-1, also had a synergistic effect on proliferation in mature hCO (FIG. 11E). Together, these data confirm our previous finding that dual inhibition of MST1 and GSK3 induces robust cell cycle re-entry. Notably, we also found considerable negative impacts on force of contraction with all small molecule inhibitors of GSK3 but not MST1 (FIG. 11F) and these effects were not reversible (FIG. 11G,H). This further demonstrates that inhibition of contraction is a consequence of GSK3 inhibition rather than activation of proliferation per se.

We analysed the activation of proliferation with compound 6.28 and the combination of CHIR99021 and compound 51 in comparison to compound 3 and 65 (FIG. 11I). There was a core overlap of 11 proteins (and a trend to increased AURKB) (FIG. 5A). These included 7 proteins in a cell cycle network, 2 proteins in the mevalonate/cholesterol pathway and 2 nuclear transporters (FIG. 5A). While these proteins were induced by the compounds and combinations that were capable of activating proliferation: compounds 3, 65, CHIR99021+compound 51 and compound 6.28 (FIG. 5B), only the “cell cycle network” proteins were induced by CHIR99021 alone and only the mevalonate enzymes were induced by compound 51 alone (FIG. 5B). This suggests that this entire network is required to drive proliferation as either CHIR99021 (GSK3i) or compound 51 (MST1i) failed to activate proliferation individually (FIG. 11). Compound 63 activated most proteins in the proliferation signature, but not as robustly as the other treatments (FIG. 5B), consistent its inability to drive a robust proliferation response. Compounds 51 and compound 65 were the most robust inducers of the mevalonate pathway and induced multiple enzymes (“cholesterol biosynthetic process”) (FIG. 5C). This indicates that cell cycle induction is associated with simultaneous activation of both a cell cycle network and the mevalonate pathway. Repression of cell cycle networks during cardiac maturation has been well studied, but less is known about the mevalonate pathway. We therefore analysed RNA-seq profiling data during the postnatal maturation window and assessed key mevalonate enzymes HMGCR, HMGCS1, CYP51A1 (Cyp51 in mouse) and SQLE. All these enzymes significantly decrease during both mouse (Quaife-Ryan et al., 2017) and human heart development (Kuppusamy et al., 2015; Mills et al., 2017a) during maturation in vivo (FIG. 5D). There is also increased expression of HMGCR and SQLE in regenerating adult mouse hearts following overexpression of constitutively active YAP1 (FIG. 5E). Taken together, these findings suggest that both cell cycle activation and the mevalonate pathway are required in proliferative cardiomyocytes (FIG. 5F).

### The Mevalonate Pathway Controls Cardiomyocyte Proliferation

We next determined whether the mevalonate pathway is functionally required for cardiomyocyte proliferation. Initial experiments were performed in immature proliferative 2D hPSC-CM (FIG. 6A) using high content imaging (FIG. 6B) and image cytometry analysis (FIG. 13A). HMGCR mediated production of cholesterol metabolites was inhibited with 10 μM simvastatin for 3 days. Under these conditions, proliferation of cardiomyocytes was markedly reduced (FIG. 6C) resulting in a decreased number of cardiomyocytes (FIG. 6D). We also found that simvastatin decreased cardiomyocyte size (FIG. 6E). Together this suggests that the mevalonate pathway enhances cardiomyocyte growth.

In order to determine which metabolites in the mevalonate pathway are critical for proliferation, we performed experiments over 24 hours to enable more precise control of metabolites and to measure the primary effects of the manipulations. In these experiments, the mevalonate pathway was inhibited using 10 μM simvastatin, and then different metabolites were provided within the medium (highlighted in red in FIG. 6F). Blocking cholesterol biosynthesis with simvastatin reduced proliferation in 2D cardiomyocytes at 24 h (FIG. 6G), but to a lesser extent than 3 days (FIG. 6C). In addition, there we no profound changes in cardiomyocyte number (FIG. 13B) or size (FIG. 13C) at this earlier time-point, thus enabling the testing of the rescue metabolites without confounding secondary effects. Interestingly, not all the downstream metabolites could rescue the inhibition of the mevalonate pathway. Consistent with previous publications (Agabiti et al., 2017; Sorrentino et al., 2014), the addition of mevalonate or geranylgeranyl pyrophosphate, but not squalene (FIG. 6G), rescued proliferation, demonstrating a requirement for mevalonate/prenylation components for cardiomyocyte proliferation. Similar to a previous report (Agabiti et al., 2017), farnesyl pyrophosphate was incapable of rescuing proliferation, potentially due to tight regulatory and negative feedback mechanisms acting on this pathway (Pool et al., 2018). This suggests that it is the intermediate metabolites that mediate the proliferative effects of the mevalonate pathway.

Given that several metabolites of the mevalonate pathway are required for post-translational modifications such as prenylation, we next performed targeted data extraction of our proteomic analysis (Gillet et al., 2012) for the addition of geranylgeranyl or farnesyl groups. We identified key targets of farnesylation, including NAP1L1 at Cys-388, which was also shown to be farnesylated in a prior study (Kho et al., 2004), and NAP1L4 at Cys-383, which we have identified as a novel farnesylated protein (FIG. 15C). Farnesylation of both proteins was increased with compound 65 while compound 3 only increased farnesylation of NAP1L1 (FIG. 15D,E), Importantly, both of these proteins are required for nucleosome assembly during DNA replication (Al Adhami et al., 2015; Qiao et al., 2018; Rodriguez et al., 2000; Schimmack et al., 2014; Yan et al., 2016), providing further evidence that the mevalonate pathway regulates cell proliferation by modulating prenylation of proteins controlling cell cycle (Charron et al., 2013; Kho et al., 2004).

The requirement of the mevalonate pathway for proliferation was next assessed in mature hCO treated with pro-proliferative compounds (FIG. 6H). The proliferative response to compounds 65, 3 and compound 6.28 was abolished when the mevalonate pathway was inhibited with 10 μM simvastatin (FIG. 6I). We also confirmed that 10 μM simvastatin abolished proliferation specifically in cardiomyocytes in hCO treated with compound 3 (FIG. 6J), without significant changes in cardiomyocyte size (FIG. 13D). Together, these data confirm the mevalonate pathway is a core component of the “proliferation signature” that is activated by all pro-proliferative compounds identified in this study.

Interestingly, mevalonate is not only required for proliferation, but the addition of mevalonate exerts control over proliferation and induces proliferation in mature hCO (FIG. 15B; Fig B— n=33 from 5 experiments. ** p<0.01 using t-test).

### The Mevalonate Pathway is Required for Cardiomyocyte Proliferation in Neonatal and Adult Cardiomyocytes In Vivo

During postnatal maturation of the heart in vivo, cardiomyocytes transition from a proliferative to a non-proliferative state. To confirm the requirement of the mevalonate pathway for proliferation during this critical developmental period, we treated neonatal mice with daily simvastatin injections from P1 to P15 (FIG. 7A). Simvastatin treatment decreased cardiomyocyte proliferation (FIG. 7B) and reduced heart size (FIG. 7C) without impacting cardiomyocyte size (FIG. 7D). Therefore, consistent with our findings in vitro using immature hPSC-CM, the mevalonate pathway is required for cardiomyocyte proliferation in vivo.

Finally, we sought to determine whether compounds 3 and 65 could regulate adult cardiomyocyte proliferation in vivo and whether the mevalonate pathway is critical for these effects. Unfortunately, compounds 3 and 65 have poor pharmacokinetic properties and can only be maintained at pro-proliferative doses in vivo for a short period of time without excessive doses (>500 mg/kg multiple times per day). Furthermore, other compounds targeting these pathways do not have identical kinase profiles. For example, compound 3 is very specific for p38α/β whereas other p38 inhibitors such as compound 9 inhibit MAPKs more broadly and do not induce proliferation. Therefore, we decided to restrict our experiments to compounds 3 and 65. To test for induction of cardiomyocyte proliferation in vivo, a single dose of 10 mg/kg was emulsified in Kolliphor® HS 15 as previously described (Fan et al., 2016) and injected directly into the heart to ensure high doses were achieved (FIG. 7E). Following injection, proliferation was assessed using BrdU pulse-chase for 3 days, followed by fix-dissociation of the hearts, enabling preservation and separation of cardiomyocytes (FIG. 7F) and accurate quantification of BrdU positive cardiomyocytes (FIG. 7G). Both compounds 3 and 65 significantly increased cardiomyocyte DNA synthesis (FIG. 7H). Importantly, inhibition of the mevalonate pathway with simvastatin blocked the DNA synthesis response in adult cardiomyocytes treated with compound 3, confirming the importance of the mevalonate pathway for cardiomyocyte proliferation in vivo (FIG. 7I). Together, our data suggest that compounds 3 and 65 are capable of activating the cell cycle in mature cardiomyocytes and that the mevalonate pathway is critical for cardiomyocyte proliferation in hPSC-CM and in vivo.

### Discussion

### hCO for Drug Screening

The current study demonstrates the power of human organoid screening platforms for drug discovery applications. Functional screening in hCO was critical as it eliminated false-positives detected in traditional 2D culture formats, and also predicted potential side-effects on cardiac contractility and rhythm. By coupling high-content screening for cardiomyocyte proliferation with high-throughput proteomics, critical regulatory pathways driving human cardiomyocyte proliferation were identified, including a previously unrecognized role for the mevalonate pathway.

The use of multiple phenotypic read-outs enabled the identification of pro-proliferative compounds (and pathways) with potential cardiotoxicities. For example, although GSK3 inhibitors and adenosine receptor 2A activators consistently activated proliferation in the immature hCO, they also reduced contractile force (sometimes to completely non-contractile levels). This finding is consistent with a recent report, which demonstrated that in vivo knockout of GSK3α and GSK3β in adult mouse cardiomyocytes resulted in a large increase in proliferation rates but also had detrimental impacts on cardiomyocyte viability and the heart's contractile performance (Zhou et al., 2016). While it could be possible that all pro-regenerative therapeutics that activate proliferation impact function, this seems unlikely given that pro-regenerative factors such as miR-199a, miR-590 and activated YAP1 do not adversely impact cardiac function (Eulalio et al., 2012; Leach et al., 2017). Our study clearly demonstrates that there are several targets that can induce cardiomyocyte proliferation without impacting force, including our hit compounds 3 and 65. Together, our study highlights the utility of hCO screening platforms for coupling contractile assays with pro-proliferative assays in the drug development pipeline to identify potential therapeutic targets with minimal cardiotoxicities for clinical translation.

### Mechanisms of Action

The hit compounds (in addition to GSK3 inhibition and MST1 inhibition) all regulate different targets and activate distinct cell cycle networks (FIG. 12). However, there is a core proliferation signature comprising a set of cell cycle proteins and the mevalonate pathway (FIG. 5A). As YAP1 is one of the most potent drivers of cardiomyocyte proliferation, we determined whether induction of proliferation was correlated with activation of YAP1. MST1 inhibition with compound 51 resulted in activation of the well characterized YAP target genes CTGF and AXL (Zanconato et al., 2015), which are repressed in hCO during maturation (Mills et al., 2017a) (FIG. 14A). However, other treatments that inhibit MST1 (compound 6.28) did not result in activation of these genes (FIG. 14A). This may be due to changes in transcriptional complexes with combinatorial treatments, which has been previously shown in both cancer cells (Rosenbluh et al., 2012) and the heart (Heallen et al., 2011; Tao et al., 2016). In contrast, the direct YAP1 target AURKB (Morikawa et al., 2015) was increased in treatments activating proliferation (FIG. 14A). We therefore stained hCO treated with different pro-proliferative compounds and found that YAP1 was activated in the nucleus under all pro-proliferative conditions (FIG. 14B). Therefore, the regulation of YAP1 targets appears to be highly context-dependent (Tao et al., 2016).

The lack of full cell cycle activation with MST1 inhibition is at odds with some reports in the literature that suggest that genetic inhibition of the Hippo pathway is sufficient to mediate adult cardiomyocyte proliferation (Leach et al., 2017). We therefore examined MST1 inhibitors in more detail and found that both compound 51 and XMU-XP-1 not only inhibit MST1, but also have off-target effects on cell cycle proteins (compound 51—CCNB1, CDK1, CCNA2, CDK2 and CCNA1 and XMU-XP-1-PLK4 and AURKB (Fan et al., 2016)). Therefore, the lack of proliferation following MST1 inhibition may be due to off-target effects of the inhibitors.

The current study adds to a growing body of evidence suggesting alterations in cardiomyocyte metabolism may be a cause rather than a consequence of cardiomyocyte cell cycle arrest. Controlling cellular metabolism is emerging as a key strategy in the fight against cancer (Vander Heiden et al., 2009), but the same pathways may conversely be required for cardiac regeneration. Our findings suggest that the mevalonate pathway is required for proliferation in vitro and in vivo under multiple experimental conditions. The importance of the mevalonate pathway for cell proliferation is supported by numerous studies in the cancer literature, which have shown that inhibition of key enzymes including HMGCR (Ashida et al., 2017; Clendening et al., 2010), HMGCS1 (Ashida et al., 2017; Clendening et al., 2010), CYP51A1 (Hargrove et al., 2016), and SQLE (Brown et al., 2016; Sui et al., 2015) all result in reduced proliferation, and Cyp5l has been previously shown to regulate proliferation in the heart (Keber et al., 2011). Thus, our study adds to the growing body of evidence that metabolism is a key controller of proliferation.

We show that it is the intermediate metabolites including mevalonate and geranylgeranyl pyrophosphate, rather than the downstream products such as squalene, that are critical for activation of proliferation. In previous publications it has been shown that mevalonate and geranylgeranyl pyrophosphate can regulate a number of different biological processes through prenylation of G-proteins or metabolic proteins including YAP1 activation (Sorrentino et al., 2014), metabolism through CoQ (Fazakerley et al., 2018), and autophagy (Miettinen and Bjorklund, 2015). Considering YAP activation is observed in all pro-proliferative conditions, how intermediate metabolites of the mevalonate pathway, and in particular protein prenylation, regulate cardiomyocyte proliferation warrants further investigation.

### Implications for Clinical Trials

Statin use during the first trimester of pregnancy has been recently linked with congenital heart defects in children (Hekimian et al., 2018). Given that defects in cardiomyocyte proliferation can contribute to congenital heart disease, the potential mechanistic link between statin use, cardiomyocyte proliferation and cardiac developmental anomalies warrants further investigation. These findings also have important implications for the future design of clinical trials for cardiac regeneration as over 27% of people over the age of 40 take statins (Salami et al., 2017), which may block regenerative therapeutics. As withdrawing statin use may pose a health risk, this could be mitigated by use of PCSK9 inhibitors (Stoekenbroek et al., 2018), which do not directly act on the mevalonate pathway.

### SUMMARY

Our findings demonstrate that pluripotent stem cell-derived hCO provide a powerful and highly predictive model for cardiac drug discovery that has the potential to identify new therapeutic targets, minimise potential side-effects and reveal previously unappreciated biological mechanisms of action.

TABLE 1

| | | | | | | | | 2D | 2D | 2D | | | | | | |
|---|---|---|---|---|---|---|---|---|---|---|---|---|---|---|---|---|
| | | | | | | | | Screen | Screen | Screen | | hCO | hCO | hCO | | |
| | | | | | | | | (AZ) | (AZ) | (AZ) | | Screen | Screen | Screen | | |
| | | | | | | | | 0.1 uM | 1 uM | 10 uM | 2D | 0.1 uM | 1 uM | 10 uM | hCO | Gene |
| | | | | | | | | Prolif | Prolif | Prolif | Screen | Prolif | Prolif | Prolif | Screen | Symbol |
| | | | | | | | | Activation | Activation | Activation | Max | Activation | Activation | Activation | Max | (targets <1 |
| | | | | | | | | EdU | EdU | EdU | Effect | Ki-67 | Ki-67 | Ki-67 | Effect | μM |
| Compound | MW | Formula | ClogP | HBA | HBD | PSA | RotBond | (%) | (%) | (%) | (%) | (%) | (%) | (%) | (%) | activity) |
| 1 | 457.5 | C23 H25 | 4.4 | 8 | 2 | 86.7 | 9 | 12 | 51 | 102 | 102 | 17 | 26 | −26 | 26 | CSF1R, FYN, |
| | | F2 N5 O3 | | | | | | | | | | | | | | LCK, BRAF |
| 2 | 622.7 | C25 H30 | 3.9 | 10 | 1 | 123.5 | 11 | 1 | 12 | 121 | 121 | 14 | −13 | −83 | 14 | ADAMTS5, |
| | | F4 N4 | | | | | | | | | | | | | | MMP2, |
| | | O6 S2 | | | | | | | | | | | | | | MMP13, |
| | | | | | | | | | | | | | | | | ADAM17, |
| | | | | | | | | | | | | | | | | CACNA1B |
| 3 | 503.6 | C27 H33 | 4.0 | 10 | 2 | 96.6 | 10 | −6 | 35 | 183 | 183 | 299 | 23 | 111 | 299 | MAPK14, |
| | | N7 O3 | | | | | | | | | | | | | | BRAF, |
| | | | | | | | | | | | | | | | | DDR1 |
| 4 | 537.7 | C32 H35 | 6.1 | 8 | 1 | 72.5 | 9 | 24 | 46 | −65 | 46 | −21 | 2 | −16 | 2 | MAPK14, |
| | | N5 O3 | | | | | | | | | | | | | | MAPK11 |
| 5 | 426.5 | C25 H22 | 6.0 | 7 | 2 | 84.8 | 8 | 26 | 19 | 134 | 134 | −28 | −43 | −47 | −28 | MERTK, |
| | | N4 O3 | | | | | | | | | | | | | | CHEK2, |
| | | | | | | | | | | | | | | | | EGFR, |
| | | | | | | | | | | | | | | | | ERBB4, LCK, |
| | | | | | | | | | | | | | | | | LYN, |
| | | | | | | | | | | | | | | | | MAP3K9, |
| | | | | | | | | | | | | | | | | ATM, AXL, |
| | | | | | | | | | | | | | | | | MAP2K1, |
| | | | | | | | | | | | | | | | | BLK, WNK3, |
| | | | | | | | | | | | | | | | | WNK2, SYK, |
| | | | | | | | | | | | | | | | | EIF2AK3 |
| 6 | 539.6 | C30 H33 | 3.4 | 10 | 1 | 83.3 | 9 | 11 | 101 | 29 | 101 | −18 | 8 | −1 | 8 | MAPK14, |
| | | N7 O3 | | | | | | | | | | | | | | MAPK1, |
| | | | | | | | | | | | | | | | | MAPK11, |
| | | | | | | | | | | | | | | | | MAP2K1, |
| | | | | | | | | | | | | | | | | BRAF |
| 7 | 424.9 | C21 H21 | 3.5 | 8 | 2 | 86.9 | 7 | | | | | −13 | 3 | −3 | 3 | MAPK11 |
| | | Cl N6 O2 | | | | | | | | | | | | | | |
| 8 | 513.4 | C19 H25 | 1.4 | 10 | 1 | 115.5 | 10 | 9 | 4 | 50 | 50 | 39 | 141 | −10 | 141 | MMP1, |
| | | Br N6 | | | | | | | | | | | | | | MMP2, |
| | | O4 S | | | | | | | | | | | | | | MMP3, |
| | | | | | | | | | | | | | | | | MMP7, |
| | | | | | | | | | | | | | | | | MMP8, |
| | | | | | | | | | | | | | | | | MMP13, |
| | | | | | | | | | | | | | | | | MMP14, |
| | | | | | | | | | | | | | | | | MMP19, |
| | | | | | | | | | | | | | | | | ADAM17, |
| | | | | | | | | | | | | | | | | ADAMTS4 |
| 9 | 377.4 | C21 H16 | 3.0 | 4 | 1 | 52.8 | 4 | 30 | 11 | 158 | 158 | −31 | 16 | 44 | 44 | CSNK1A1L, |
| | | F N3 O S | | | | | | | | | | | | | | MAPK14, |
| | | | | | | | | | | | | | | | | CSNK1A1, |
| | | | | | | | | | | | | | | | | CSNK1D, |
| | | | | | | | | | | | | | | | | CSNK1E, |
| | | | | | | | | | | | | | | | | CSNK1G2, |
| | | | | | | | | | | | | | | | | CSNK1G3, |
| | | | | | | | | | | | | | | | | CSNK2A1, |
| | | | | | | | | | | | | | | | | EGFR, |
| | | | | | | | | | | | | | | | | CSNK2A3, |
| | | | | | | | | | | | | | | | | PACSIN1, |
| | | | | | | | | | | | | | | | | LCK, DDR2, |
| | | | | | | | | | | | | | | | | CSNK1G1, |
| | | | | | | | | | | | | | | | | MAPK1, |
| | | | | | | | | | | | | | | | | MAPK11, |
| | | | | | | | | | | | | | | | | MAPK9, |
| | | | | | | | | | | | | | | | | MAPK10, |
| | | | | | | | | | | | | | | | | MAP2K1, |
| | | | | | | | | | | | | | | | | NUCKS1, |
| | | | | | | | | | | | | | | | | BRAF, |
| | | | | | | | | | | | | | | | | MAPKAPK2 |
| 10 | 281.4 | C16 H19 | 3.4 | 5 | 2 | 61.9 | 2 | 9 | 108 | 224 | 224 | 9 | 31 | 13 | 31 | IRAK3, |
| | | N5 | | | | | | | | | | | | | | CSF1R, CSK, |
| | | | | | | | | | | | | | | | | CSNK1D, |
| | | | | | | | | | | | | | | | | EGFR, |
| | | | | | | | | | | | | | | | | EPHA2, |
| | | | | | | | | | | | | | | | | EPHB3, |
| | | | | | | | | | | | | | | | | EPHB4, |
| | | | | | | | | | | | | | | | | FGFR1, |
| | | | | | | | | | | | | | | | | FLT1, |
| | | | | | | | | | | | | | | | | SIK2, SIK3, |
| | | | | | | | | | | | | | | | | FYN, KDR, |
| | | | | | | | | | | | | | | | | KIT, LCK, |
| | | | | | | | | | | | | | | | | LYN, DDR2, |
| | | | | | | | | | | | | | | | | MAPK11, |
| | | | | | | | | | | | | | | | | RET, SRC, |
| | | | | | | | | | | | | | | | | AURKA, |
| | | | | | | | | | | | | | | | | BTK, TEK, |
| | | | | | | | | | | | | | | | | TGFBR1, |
| | | | | | | | | | | | | | | | | YES1, |
| | | | | | | | | | | | | | | | | RIPK2, |
| | | | | | | | | | | | | | | | | AURKB, |
| | | | | | | | | | | | | | | | | ACVR2B, |
| | | | | | | | | | | | | | | | | ACVRL1 |
| 11 | 253.3 | C10 H11 | 0.6 | 6 | 3 | 102.8 | 2 | −4 | 16 | 58 | 58 | 0 | −12 | −15 | 0 | |
| | | N3 O3 S | | | | | | | | | | | | | | |
| 12 | 381.4 | C22 H24 | 3.8 | 5 | 1 | 52.3 | 8 | 13 | 76 | 54 | 76 | 6 | 27 | 25 | 27 | CHRM1, |
| | | F N3 O2 | | | | | | | | | | | | | | HTR1A, |
| | | | | | | | | | | | | | | | | OPRM1 |
| 13 | 383.5 | C21 H25 | 4.2 | 5 | 2 | 76.9 | 9 | 26 | 42 | 196 | 196 | 54 | 31 | 119 | 119 | PRKACA, |
| | | N3 O2 S | | | | | | | | | | | | | | MAPK1 |
| 14 | 414.5 | C22 H26 | 3.6 | 6 | 0 | 56.5 | 7 | 13 | 138 | 248 | 248 | 9 | −4 | 33 | 33 | CACNA1C |
| | | N2 O4 S | | | | | | | | | | | | | | |
| 15 | 153.2 | C8 H11 | 0.2 | 3 | 4 | 73.7 | 2 | −4 | 34 | 155 | 155 | −24 | −7 | −26 | −7 | DRD2, |
| | | N O2 | | | | | | | | | | | | | | SLC6A2, |
| | | | | | | | | | | | | | | | | CACNA1C |
| 16 | 406.4 | C23 H22 | 5.1 | 7 | 2 | 101.8 | 9 | | | | | 27 | 19 | 15 | 27 | GCK, RARA, |
| | | N2 O5 | | | | | | | | | | | | | | RARB |
| 17 | 298.3 | C16 H14 | 4.7 | 6 | 1 | 76.4 | 3 | 20 | 53 | 87 | 87 | −26 | −48 | −56 | −26 | CDK8, |
| | | N2 O4 | | | | | | | | | | | | | | LRRK2, |
| | | | | | | | | | | | | | | | | AHR, |
| | | | | | | | | | | | | | | | | NFE2L2, |
| | | | | | | | | | | | | | | | | CLK4, |
| | | | | | | | | | | | | | | | | SLC6A4, |
| | | | | | | | | | | | | | | | | CCNC |
| 18 | 527 | C27 H31 | 6.0 | 9 | 1 | 80.8 | 12 | 1 | 63 | 211 | 211 | −25 | −12 | −44 | −12 | CSF1R, CSK, |
| | | Cl N4 O5 | | | | | | | | | | | | | | EGFR, |
| | | | | | | | | | | | | | | | | EPHB4, |
| | | | | | | | | | | | | | | | | ABL1, FYN, |
| | | | | | | | | | | | | | | | | ABL2, |
| | | | | | | | | | | | | | | | | LCK, LYN, |
| | | | | | | | | | | | | | | | | SRC, |
| | | | | | | | | | | | | | | | | TGFBR1, |
| | | | | | | | | | | | | | | | | ACVRL1 |
| 19 | 551.7 | C31 H41 | 2.0 | 7 | 1 | 90.2 | 13 | 15 | 21 | 131 | 131 | 26 | 10 | 0 | 26 | CHRM2, |
| | | N3 O4 S | | | | | | | | | | | | | | CCR5, |
| | | | | | | | | | | | | | | | | CYP3A4 |
| 20 | 384.3 | C18 H19 | 5.3 | 5 | 1 | 68.7 | 4 | 245 | 232 | 257 | 257 | 213 | 228 | 159 | 228 | CYP2C9, |
| | | Cl2 N O4 | | | | | | | | | | | | | | NR3C2 |
| 21 | 267.2 | C10 H13 | −2.2 | 9 | 5 | 135.4 | 3 | 12 | 10 | 52 | 52 | 91 | 192 | 159 | 192 | CLK1, |
| | | N5 O4 | | | | | | | | | | | | | | ADORA1, |
| | | | | | | | | | | | | | | | | ADORA2A, |
| | | | | | | | | | | | | | | | | ADORA3, |
| | | | | | | | | | | | | | | | | SPECC1L |
| 22 | 502.4 | C27 H24 | 7.0 | 5 | 1 | 51.9 | 6 | 14 | −12 | 313 | 313 | | | | | |
| | | Br N3 O2 | | | | | | | | | | | | | | |
| 23 | 409.4 | C22 H23 | 4.0 | 3 | 1 | 42.0 | 9 | | 100 | 113 | 113 | 50 | −15 | −15 | 50 | CYP2D6, |
| | | F4 N O2 | | | | | | | | | | | | | | OPRM1 |
| 24 | 146.2 | C7 H16 | −3.5 | 3 | 0 | 27.1 | 3 | 9 | 42 | 10 | 42 | −29 | −65 | −54 | −29 | CHRM1, |
| | | N O2 | | | | | | | | | | | | | | CHRM2, |
| | | | | | | | | | | | | | | | | CHRM3, |
| | | | | | | | | | | | | | | | | CHRM4, |
| | | | | | | | | | | | | | | | | CHRM5, |
| | | | | | | | | | | | | | | | | P2RY2 |
| 25 | 501 | C26 H29 | 4.9 | 6 | 1 | 71.2 | 10 | | | | | 130 | 57 | 155 | 155 | |
| | | Cl N2 | | | | | | | | | | | | | | |
| | | O4 S | | | | | | | | | | | | | | |
| 26 | 133.2 | C9 H11 | 1.5 | 1 | 2 | 28.0 | 1 | 4 | 19 | 132 | 132 | 19 | −1 | 47 | 47 | |
| | | N | | | | | | | | | | | | | | |
| 27 | 453.5 | C21 H23 | 2.3 | 10 | 3 | 130.8 | 6 | 16 | 136 | −1 | 136 | 285 | 110 | −98 | 285 | CDK2, |
| | | N7 O3 S | | | | | | | | | | | | | | CDK5, |
| | | | | | | | | | | | | | | | | DYRK1A, |
| | | | | | | | | | | | | | | | | EGFR, AHR, |
| | | | | | | | | | | | | | | | | FLT1, ALPI, |
| | | | | | | | | | | | | | | | | ALPL, |
| | | | | | | | | | | | | | | | | ALPP, |
| | | | | | | | | | | | | | | | | ALPPL2, |
| | | | | | | | | | | | | | | | | HIPK2, |
| | | | | | | | | | | | | | | | | GSK3A, |
| | | | | | | | | | | | | | | | | GSK3B, |
| | | | | | | | | | | | | | | | | MAP3K11, |
| | | | | | | | | | | | | | | | | CDK16, |
| | | | | | | | | | | | | | | | | PIM1, ATR, |
| | | | | | | | | | | | | | | | | PRKCI, |
| | | | | | | | | | | | | | | | | PRKD1, |
| | | | | | | | | | | | | | | | | MAPK8, |
| | | | | | | | | | | | | | | | | CCNB1IP1, |
| | | | | | | | | | | | | | | | | MAP4K2, |
| | | | | | | | | | | | | | | | | MAP3K7, |
| | | | | | | | | | | | | | | | | DYRK3, |
| | | | | | | | | | | | | | | | | DYRK2, |
| | | | | | | | | | | | | | | | | KIAA1804, |
| | | | | | | | | | | | | | | | | CCNB3, |
| | | | | | | | | | | | | | | | | RIPK2, |
| | | | | | | | | | | | | | | | | CCNA2, |
| | | | | | | | | | | | | | | | | CCNA1, |
| | | | | | | | | | | | | | | | | CCNB1, |
| | | | | | | | | | | | | | | | | CCNE1, |
| | | | | | | | | | | | | | | | | CCNE2, |
| | | | | | | | | | | | | | | | | CDK1 |
| 28 | 341.3 | C17 H11 | 3.2 | 8 | 5 | 160.8 | 3 | | | | | 62 | 4 | 17 | 62 | ADRBK1, |
| | | N O7 | | | | | | | | | | | | | | FEN1 |
| 29 | 326.8 | C18 H19 | 4.5 | 4 | 2 | 42.6 | 5 | −1 | 18 | 116 | 116 | −10 | −24 | 68 | 68 | PRKACA, |
| | | Cl N4 | | | | | | | | | | | | | | ROCK1, |
| | | | | | | | | | | | | | | | | ROCK2 |
| 30 | 512 | C24 H22 | 2.8 | 9 | 1 | 104.1 | 5 | −3 | 76 | 114 | 114 | 45 | 33 | −33 | 45 | F10 |
| | | Cl N5 | | | | | | | | | | | | | | |
| | | O4 S | | | | | | | | | | | | | | |
| 31 | 366.5 | C22 H26 | 3.8 | 5 | 0 | 37.8 | 10 | −1 | 10 | 59 | 59 | 13 | −20 | −50 | 13 | |
| | | N2 O3 | | | | | | | | | | | | | | |
| 32 | 305.8 | C16 H16 | 2.4 | 4 | 4 | 83.2 | 3 | 71 | 134 | 157 | 157 | 24 | −67 | −55 | 24 | DRD1 |
| | | Cl N O3 | | | | | | | | | | | | | | |
| 33 | 351.4 | C22 H25 | 6.3 | 4 | 2 | 73.4 | 4 | 31 | 66 | 66 | 66 | 13 | 62 | 5 | 62 | RARA, |
| | | N O3 | | | | | | | | | | | | | | RARB, |
| | | | | | | | | | | | | | | | | RARG |
| 34 | 464.5 | C26 H28 | 4.9 | 8 | 2 | 110.6 | 11 | −1 | 67 | 16 | 67 | 29 | 64 | 62 | 64 | GCK, IGF1R, |
| | | N2 O6 | | | | | | | | | | | | | | RARA, |
| | | | | | | | | | | | | | | | | RARB, |
| | | | | | | | | | | | | | | | | RARG, |
| | | | | | | | | | | | | | | | | EIF2AK3 |
| 35 | 527.7 | C31 H37 | 6.9 | 8 | 2 | 79.3 | 10 | 12 | 45 | 109 | 109 | 10 | 3 | 16 | 16 | MAPK14, |
| | | N5 O3 | | | | | | | | | | | | | | CYP2C9, |
| | | | | | | | | | | | | | | | | HSF1, KDR, |
| | | | | | | | | | | | | | | | | MAPK1, |
| | | | | | | | | | | | | | | | | MAPK11, |
| | | | | | | | | | | | | | | | | MAPK9, |
| | | | | | | | | | | | | | | | | MAPK10, |
| | | | | | | | | | | | | | | | | MAPK12, |
| | | | | | | | | | | | | | | | | MAP3K7, |
| | | | | | | | | | | | | | | | | MAPKAPK2 |
| 36 | 705.6 | C35 H38 | 6.0 | 12 | 0 | 84.7 | 14 | 6 | 69 | 138 | 138 | 5 | −24 | −12 | 5 | CYP3A4 |
| | | Cl2 N8 O4 | | | | | | | | | | | | | | |
| 37 | 414.6 | C19 H22 | 4.1 | 7 | 4 | 113.9 | 9 | −11 | 15 | 294 | 294 | −18 | −6 | 111 | 111 | CX3CR1 |
| | | N6 O S2 | | | | | | | | | | | | | | |
| 38 | 315.5 | C19 H25 | 4.5 | 2 | 1 | 24.1 | 7 | | 100 | 288 | 288 | 32 | −36 | 16 | 32 | DRD2, |
| | | N O S | | | | | | | | | | | | | | HTR1A |
| 39 | 284.4 | C19 H24 O2 | 4.0 | 2 | 0 | 27.4 | 1 | −3 | 30 | 45 | 45 | 24 | 20 | 25 | 25 | |
| 40 | 405.5 | C21 H31 | −1.8 | 8 | 5 | 143.7 | 13 | 7 | 23 | 97 | 97 | 43 | 21 | 69 | 69 | |
| | | N3 O5 | | | | | | | | | | | | | | |
| 41 | 540.6 | C23 H27 | 2.0 | 10 | 4 | 138.1 | 12 | −33 | 96 | 217 | 217 | 6 | 30 | 102 | 102 | ADORA3, |
| | | F3 N6 | | | | | | | | | | | | | | FAAH, |
| | | O4 S | | | | | | | | | | | | | | P2RY12 |
| 42 | 496.6 | C23 H20 | 5.0 | 7 | 1 | 80.7 | 6 | 0 | 2 | 206 | 206 | 105 | 6 | 105 | 105 | |
| | | N4 O3 | | | | | | | | | | | | | | |
| | | S3 | | | | | | | | | | | | | | |
| 43 | 419.5 | C24 H25 | 3.3 | 7 | 2 | 93.5 | 8 | 1 | −11 | 104 | 104 | −27 | 95 | 80 | 95 | CNR1, |
| | | N3 O4 | | | | | | | | | | | | | | CNR2 |
| 44 | 369.3 | C19 H26 | 4.9 | 3 | 0 | 20.6 | 7 | 7 | 12 | 93 | 93 | 87 | 5 | 199 | 199 | GHSR, |
| | | Cl2 N2 | | | | | | | | | | | | | | OPRK1, |
| | | O | | | | | | | | | | | | | | OPRM1 |
| 45 | 474.6 | C29 H35 | 4.0 | 5 | 2 | 49.5 | 10 | −14 | 11 | 219 | 219 | 46 | 68 | 154 | 154 | KCNH2, |
| | | F N4 O | | | | | | | | | | | | | | OPRD1, |
| | | | | | | | | | | | | | | | | OPRM1 |
| 46 | 360.4 | C21 H28 | 1.4 | 5 | 3 | 104.2 | 2 | | | | | 187 | 122 | 141 | 187 | MALT1, |
| | | O5 | | | | | | | | | | | | | | NR3C1, |
| | | | | | | | | | | | | | | | | APOE, |
| | | | | | | | | | | | | | | | | NR3C2, |
| | | | | | | | | | | | | | | | | NFE2L2, |
| | | | | | | | | | | | | | | | | RARG |
| 47 | 413.5 | C24 H23 | 3.4 | 7 | 2 | 86.3 | 7 | 47 | 151 | 239 | 239 | 4 | 28 | −6 | 28 | MAP4K1, |
| | | N5 O2 | | | | | | | | | | | | | | MAPK14, |
| | | | | | | | | | | | | | | | | CSF1R, |
| | | | | | | | | | | | | | | | | CYP3A4, |
| | | | | | | | | | | | | | | | | EPHA5, |
| | | | | | | | | | | | | | | | | EPHB1, |
| | | | | | | | | | | | | | | | | EPHB4, |
| | | | | | | | | | | | | | | | | FGFR1, |
| | | | | | | | | | | | | | | | | FGFR3, |
| | | | | | | | | | | | | | | | | FGFR2, |
| | | | | | | | | | | | | | | | | FLT1, SIK2, |
| | | | | | | | | | | | | | | | | SIK3, ABL1, |
| | | | | | | | | | | | | | | | | FYN, ABL2, |
| | | | | | | | | | | | | | | | | JAK2, KDR, |
| | | | | | | | | | | | | | | | | KIT, LCK, |
| | | | | | | | | | | | | | | | | LYN, |
| | | | | | | | | | | | | | | | | MAP3K1, |
| | | | | | | | | | | | | | | | | MAP3K9, |
| | | | | | | | | | | | | | | | | NFE2L2, |
| | | | | | | | | | | | | | | | | NTRK1, |
| | | | | | | | | | | | | | | | | DDR2, |
| | | | | | | | | | | | | | | | | MINK1, |
| | | | | | | | | | | | | | | | | NIN, |
| | | | | | | | | | | | | | | | | PDGFRB, |
| | | | | | | | | | | | | | | | | PTK6, RAF1, |
| | | | | | | | | | | | | | | | | RET, BLK, |
| | | | | | | | | | | | | | | | | BMX, SRC, |
| | | | | | | | | | | | | | | | | BRAF, BTK, |
| | | | | | | | | | | | | | | | | TGFBR1, |
| | | | | | | | | | | | | | | | | TYRO3, |
| | | | | | | | | | | | | | | | | YES1, |
| | | | | | | | | | | | | | | | | ACVR1B, |
| | | | | | | | | | | | | | | | | ACVR2B, |
| | | | | | | | | | | | | | | | | ACVRL1 |
| 48 | 466.4 | C24 H17 | 4.0 | 7 | 3 | 107.6 | 5 | 25 | 61 | 115 | 115 | −19 | 6 | 55 | 55 | CSF1R, |
| | | F3 N4 O3 | | | | | | | | | | | | | | RAF1, BRAF |
| 49 | 490.1 | C28 H28 | 5.3 | 4 | 1 | 44.3 | 8 | | | | | 18 | −22 | −7 | 18 | |
| | | Cl N3 O | | | | | | | | | | | | | | |
| | | S | | | | | | | | | | | | | | |
| 50 | 541.6 | C23 H27 | 4.5 | 9 | 1 | 85.2 | 8 | −8 | 20 | 133 | 133 | 18 | 36 | 189 | 189 | CDK2, |
| | | F4 N7 | | | | | | | | | | | | | | GSK3A, |
| | | O2 S | | | | | | | | | | | | | | GSK3B, |
| | | | | | | | | | | | | | | | | CCNE1, |
| | | | | | | | | | | | | | | | | CCNE2 |
| 51 | 388.4 | C19 H18 | 4.1 | 5 | 4 | 85.3 | 6 | | | | | 47 | 108 | −71 | 108 | CHEK1, |
| | | F2 N4 O | | | | | | | | | | | | | | MAP4K1, |
| | | S | | | | | | | | | | | | | | CSF1R, |
| | | | | | | | | | | | | | | | | EGFR, |
| | | | | | | | | | | | | | | | | AKT1, SIK2, |
| | | | | | | | | | | | | | | | | SIK3, GHSR, |
| | | | | | | | | | | | | | | | | TBK1, |
| | | | | | | | | | | | | | | | | JAK2, JAK3, |
| | | | | | | | | | | | | | | | | KDR, LCK, |
| | | | | | | | | | | | | | | | | NFKB1, |
| | | | | | | | | | | | | | | | | NTRK1, |
| | | | | | | | | | | | | | | | | PAK1, |
| | | | | | | | | | | | | | | | | PAK2, |
| | | | | | | | | | | | | | | | | PDPK1, |
| | | | | | | | | | | | | | | | | AXL, |
| | | | | | | | | | | | | | | | | MAPK1, |
| | | | | | | | | | | | | | | | | PTK2, RET, |
| | | | | | | | | | | | | | | | | BMX, SRC, |
| | | | | | | | | | | | | | | | | BRAF, STK4, |
| | | | | | | | | | | | | | | | | MAP3K7, |
| | | | | | | | | | | | | | | | | TTK, |
| | | | | | | | | | | | | | | | | MAP4K3, |
| | | | | | | | | | | | | | | | | ACVR2B |
| 52 | 371.4 | C21 H23 | 4.8 | 4 | 1 | 43.7 | 5 | 32 | 61 | 45 | 61 | −8 | 3 | −14 | 3 | TRPV1 |
| | | F2 N3 O | | | | | | | | | | | | | | |
| 53 | 382.4 | C20 H22 | 3.3 | 8 | 1 | 96.9 | 7 | 6 | 43 | 141 | 141 | −2 | 2 | 11 | 11 | TRPV1 |
| | | N4 O4 | | | | | | | | | | | | | | |
| 54 | 569.5 | C25 H27 | 5.4 | 8 | 1 | 97.0 | 10 | −1 | 34 | 230 | 230 | −46 | −12 | −38 | −12 | CNR1 |
| | | Cl2 F N4 | | | | | | | | | | | | | | |
| | | O4 S | | | | | | | | | | | | | | |
| 55 | 380.9 | C23 H25 | 5.2 | 3 | 0 | 24.5 | 7 | 70 | 116 | 80 | 116 | −32 | 1 | 29 | 29 | SLC6A2 |
| | | Cl N2 O | | | | | | | | | | | | | | |
| 56 | 290.4 | C16 H22 | 0.7 | 5 | 4 | 92.2 | 5 | 85 | 57 | −23 | 85 | −12 | −27 | −18 | −12 | CRHR1, |
| | | N2 O3 | | | | | | | | | | | | | | GPR39 |
| 57 | 465.6 | C26 H35 | 3.3 | 8 | 2 | 78.0 | 10 | −11 | 81 | 84 | 84 | −12 | −21 | 15 | 15 | CCR5, |
| | | N5 O3 | | | | | | | | | | | | | | CCR2 |
| 58 | 399.5 | C24 H25 | 4.0 | 6 | 0 | 44.2 | 8 | | | | | 19 | −36 | 18 | 19 | CDK7, |
| | | N5 O | | | | | | | | | | | | | | IRAK3, |
| | | | | | | | | | | | | | | | | CLK1, |
| | | | | | | | | | | | | | | | | CSF1R, |
| | | | | | | | | | | | | | | | | FLT3, SIK2, |
| | | | | | | | | | | | | | | | | SIK3, KDR, |
| | | | | | | | | | | | | | | | | KIT, IRAK4, |
| | | | | | | | | | | | | | | | | CDK12, |
| | | | | | | | | | | | | | | | | AXL, BRAF, |
| | | | | | | | | | | | | | | | | ACVRL1, |
| | | | | | | | | | | | | | | | | MELK |
| 59 | 292.4 | C15 H20 | 3.2 | 4 | 3 | 78.8 | 5 | −6 | 18 | 46 | 46 | −62 | −2 | −49 | −2 | GABRR3, |
| | | N2 O2 S | | | | | | | | | | | | | | GABRA1, |
| | | | | | | | | | | | | | | | | GABRA2, |
| | | | | | | | | | | | | | | | | GABRA3, |
| | | | | | | | | | | | | | | | | GABRA4, |
| | | | | | | | | | | | | | | | | GABRA5, |
| | | | | | | | | | | | | | | | | GABRA6, |
| | | | | | | | | | | | | | | | | GABRB1, |
| | | | | | | | | | | | | | | | | GABRB2, |
| | | | | | | | | | | | | | | | | GABRB3, |
| | | | | | | | | | | | | | | | | GABRD, |
| | | | | | | | | | | | | | | | | GABRE, |
| | | | | | | | | | | | | | | | | GABRG1, |
| | | | | | | | | | | | | | | | | GABRG2, |
| | | | | | | | | | | | | | | | | GABRG3, |
| | | | | | | | | | | | | | | | | GABRP, |
| | | | | | | | | | | | | | | | | GABRR1, |
| | | | | | | | | | | | | | | | | GABRR2, |
| | | | | | | | | | | | | | | | | GABRQ, |
| | | | | | | | | | | | | | | | | LOC730017 |
| 60 | 498.6 | C25 H27 | 2.8 | 8 | 2 | 103.6 | 8 | 4 | 54 | 127 | 127 | 14 | −25 | −26 | 14 | PDE4B |
| | | F N4 O4 | | | | | | | | | | | | | | |
| | | S | | | | | | | | | | | | | | |
| 61 | 433.9 | C18 H16 | 4.0 | 9 | 3 | 118.7 | 5 | −4 | 53 | 12 | 53 | 32 | 78 | 82 | 82 | CSF1R, |
| | | Cl N5 | | | | | | | | | | | | | | CYP2C9, |
| | | O4 S | | | | | | | | | | | | | | EPHA2, |
| | | | | | | | | | | | | | | | | EPHB4, |
| | | | | | | | | | | | | | | | | DDR2, |
| | | | | | | | | | | | | | | | | CCNB1IP1, |
| | | | | | | | | | | | | | | | | BMX, SRC, |
| | | | | | | | | | | | | | | | | SYK, |
| | | | | | | | | | | | | | | | | TGFBR1, |
| | | | | | | | | | | | | | | | | TTK, DDR1, |
| | | | | | | | | | | | | | | | | CCNB3, |
| | | | | | | | | | | | | | | | | CCNB1, |
| | | | | | | | | | | | | | | | | ACVR1B, |
| | | | | | | | | | | | | | | | | AURKB, |
| | | | | | | | | | | | | | | | | ACVRL1, |
| | | | | | | | | | | | | | | | | CDK1 |
| 62 | 382.5 | C21 H26 | 3.1 | 7 | 1 | 74.1 | 6 | 47 | 123 | 279 | 279 | −67 | −65 | −80 | −65 | |
| | | N4 O3 | | | | | | | | | | | | | | |
| 63 | 417.9 | C22 H28 | 2.7 | 6 | 3 | 89.1 | 7 | 17 | −11 | 65 | 65 | 141 | 118 | 309 | 309 | P2RX7 |
| | | Cl N3 | | | | | | | | | | | | | | |
| | | O3 | | | | | | | | | | | | | | |
| 64 | 489.6 | C28 H41 | 5.1 | 5 | 0 | 39.1 | 11 | 33 | 50 | 244 | 244 | 21 | 13 | −16 | 21 | CNR1 |
| | | F2 N3 | | | | | | | | | | | | | | |
| | | O2 | | | | | | | | | | | | | | |
| 65 | 447.5 | C23 H21 | 3.3 | 8 | 3 | 119.6 | 6 | 48 | 76 | 171 | 171 | −38 | 96 | 121 | 121 | RIPK3, |
| | | N5 O3 S | | | | | | | | | | | | | | ACVR1C, |
| | | | | | | | | | | | | | | | | ABL1, ABL2, |
| | | | | | | | | | | | | | | | | LCK, NLK, |
| | | | | | | | | | | | | | | | | ZAK, |
| | | | | | | | | | | | | | | | | BMPR1A, |
| | | | | | | | | | | | | | | | | BMPR1B, |
| | | | | | | | | | | | | | | | | SRC, |
| | | | | | | | | | | | | | | | | TGFBR1, |
| | | | | | | | | | | | | | | | | YES1, |
| | | | | | | | | | | | | | | | | ACVR1, |
| | | | | | | | | | | | | | | | | ACVR1B, |
| | | | | | | | | | | | | | | | | ACVR2A, |
| | | | | | | | | | | | | | | | | ACVR2B, |
| | | | | | | | | | | | | | | | | ACVRL1 |
| 66 | 777.9 | C40 H47 | 2.5 | 17 | 7 | 223.7 | 17 | 28 | 27 | 64 | 64 | −49 | −44 | −7 | −7 | ADORA2A, |
| | | N11 O6 | | | | | | | | | | | | | | ADORA2B, |
| | | | | | | | | | | | | | | | | ADORA3, |
| | | | | | | | | | | | | | | | | CYP3A4, |
| | | | | | | | | | | | | | | | | SPECC1L |
| 67 | 417.5 | C25 H24 | 5.3 | 5 | 1 | 55.4 | 7 | | | | | −93 | −92 | −90 | −90 | NR3C1, |
| | | F N3 O2 | | | | | | | | | | | | | | NFE2L2 |
| 68 | 454.5 | C24 H27 | 4.8 | 8 | 3 | 98.0 | 10 | 0 | 30 | 149 | 149 | −39 | −24 | −11 | −11 | CSF1R, |
| | | F N4 O4 | | | | | | | | | | | | | | JAK2, |
| | | | | | | | | | | | | | | | | MAPK1 |
| 69 | 362.4 | C18 H18 | 2.0 | 9 | 0 | 83.0 | 5 | 52 | 109 | 5 | 109 | −3 | −22 | −14 | −3 | GRM5 |
| | | N8 O | | | | | | | | | | | | | | |
| 70 | 392.4 | C18 H22 | 2.5 | 8 | 2 | 87.5 | 5 | 58 | −11 | −53 | 58 | −30 | −13 | −38 | −13 | HSP90AA1, |
| | | F2 N6 | | | | | | | | | | | | | | HSP90AA2P |
| | | O2 | | | | | | | | | | | | | | |
| 71 | 383.5 | C23 H21 | 3.4 | 6 | 2 | 77.5 | 5 | 40 | 81 | 175 | 175 | −38 | 11 | 15 | 15 | RIPK3, |
| | | N5 O | | | | | | | | | | | | | | IRAK3, |
| | | | | | | | | | | | | | | | | LRRK2, |
| | | | | | | | | | | | | | | | | CSF1R, |
| | | | | | | | | | | | | | | | | MAPK15, |
| | | | | | | | | | | | | | | | | TNIK, FLT3, |
| | | | | | | | | | | | | | | | | PIP5K1C, |
| | | | | | | | | | | | | | | | | ABL1, |
| | | | | | | | | | | | | | | | | MKNK2, |
| | | | | | | | | | | | | | | | | DDR2, |
| | | | | | | | | | | | | | | | | MINK1, |
| | | | | | | | | | | | | | | | | NIN, |
| | | | | | | | | | | | | | | | | PDE4B, |
| | | | | | | | | | | | | | | | | PDGFRB, |
| | | | | | | | | | | | | | | | | NLK, |
| | | | | | | | | | | | | | | | | MAPK3, |
| | | | | | | | | | | | | | | | | RET, |
| | | | | | | | | | | | | | | | | BMPR1A, |
| | | | | | | | | | | | | | | | | BRAF, |
| | | | | | | | | | | | | | | | | FIP1L1, |
| | | | | | | | | | | | | | | | | PIP5K1A, |
| | | | | | | | | | | | | | | | | BRSK1, |
| | | | | | | | | | | | | | | | | DYRK2, |
| | | | | | | | | | | | | | | | | MKNK1, |
| | | | | | | | | | | | | | | | | RIPK2, |
| | | | | | | | | | | | | | | | | ACVR1, |
| | | | | | | | | | | | | | | | | MYLK3, |
| | | | | | | | | | | | | | | | | ACVR2A, |
| | | | | | | | | | | | | | | | | ACVR2B, |
| | | | | | | | | | | | | | | | | ACVRL1, |
| | | | | | | | | | | | | | | | | MAP4K4 |
| 72 | 460.5 | C26 H25 | 4.7 | 7 | 1 | 74.7 | 8 | 11 | 17 | 118 | 118 | | | | | CYP2C9, |
| | | F N4 O3 | | | | | | | | | | | | | | ALOX5, |
| | | | | | | | | | | | | | | | | NR3C1, |
| | | | | | | | | | | | | | | | | APOE |
| 73 | 419.9 | C18 H22 | 2.7 | 10 | 2 | 127.4 | 8 | −5 | 23 | 59 | 59 | 175 | 125 | 173 | 175 | F2 |
| | | Cl N7 O3 | | | | | | | | | | | | | | |
| 74 | 520.6 | C31 H32 | 7.2 | 8 | 1 | 64.4 | 10 | | | | | 3 | 12 | 7 | 12 | ALPI, ALPL, |
| | | N6 O2 | | | | | | | | | | | | | | ALPP, |
| | | | | | | | | | | | | | | | | ALPPL2 |
| 75 | 274.2 | C13 H17 | 2.9 | 2 | 2 | 37.1 | 5 | 13 | 12 | 122 | 122 | −45 | 36 | −59 | 36 | SLC6A2, |
| | | Cl2 N O | | | | | | | | | | | | | | SLC6A3, |
| | | | | | | | | | | | | | | | | SLC6A4 |
| 76 | 356.2 | C16 H10 | 3.5 | 5 | 3 | 82.2 | 0 | | | | | 28 | 127 | 179 | 179 | CDK2, |
| | | Br N3 O2 | | | | | | | | | | | | | | CDK5, |
| | | | | | | | | | | | | | | | | MAP4K1, |
| | | | | | | | | | | | | | | | | EGFR, SIK2, |
| | | | | | | | | | | | | | | | | SIK3, |
| | | | | | | | | | | | | | | | | GSK3A, |
| | | | | | | | | | | | | | | | | GSK3B, LCK |
| 77 | 501.3 | C21 H18 | 2.2 | 11 | 2 | 119.8 | 7 | 2 | −3 | 45 | 45 | 15 | 0 | 37 | 37 | GCK |
| | | Cl2 N8 | | | | | | | | | | | | | | |
| | | O3 | | | | | | | | | | | | | | |
| 78 | 490.8 | C20 H16 | 3.3 | 7 | 2 | 99.2 | 6 | −4 | −10 | 110 | 110 | 149 | 25 | 193 | 193 | MLYCD, |
| | | Cl F5 N4 | | | | | | | | | | | | | | BDKRB1 |
| | | O3 | | | | | | | | | | | | | | |
| 79 | 379.4 | C21 H18 | 3.1 | 6 | 3 | 107.6 | 5 | −8 | 11 | 47 | 47 | 40 | 18 | 32 | 40 | DGAT1 |
| | | F N3 O3 | | | | | | | | | | | | | | |
| 80 | 420.5 | C23 H28 | 3.0 | 8 | 4 | 114.3 | 11 | | | | | | | | | HIPK3, |
| | | N6 O2 | | | | | | | | | | | | | | CDK2, |
| | | | | | | | | | | | | | | | | CDK7, |
| | | | | | | | | | | | | | | | | MERTK, |
| | | | | | | | | | | | | | | | | CAMKK2, |
| | | | | | | | | | | | | | | | | PIM2, |
| | | | | | | | | | | | | | | | | MAP4K1, |
| | | | | | | | | | | | | | | | | CHEK2, |
| | | | | | | | | | | | | | | | | IRAK3, |
| | | | | | | | | | | | | | | | | CLK1, CLK2, |
| | | | | | | | | | | | | | | | | LRRK2, |
| | | | | | | | | | | | | | | | | CSNK1A1L, |
| | | | | | | | | | | | | | | | | CSNK1A1, |
| | | | | | | | | | | | | | | | | CSNK1D, |
| | | | | | | | | | | | | | | | | CSNK1E, |
| | | | | | | | | | | | | | | | | CSNK1G2, |
| | | | | | | | | | | | | | | | | CSNK1G3, |
| | | | | | | | | | | | | | | | | CSNK2A1, |
| | | | | | | | | | | | | | | | | DRD3, |
| | | | | | | | | | | | | | | | | DYRK1A, |
| | | | | | | | | | | | | | | | | EGFR, |
| | | | | | | | | | | | | | | | | HIPK1, FES, |
| | | | | | | | | | | | | | | | | FGR, FLT1, |
| | | | | | | | | | | | | | | | | FLT3, FLT4, |
| | | | | | | | | | | | | | | | | ABL1, |
| | | | | | | | | | | | | | | | | PRKD2, |
| | | | | | | | | | | | | | | | | ABL2, |
| | | | | | | | | | | | | | | | | CSNK2A3, |
| | | | | | | | | | | | | | | | | HIPK2, |
| | | | | | | | | | | | | | | | | GSK3A, |
| | | | | | | | | | | | | | | | | GSK3B, |
| | | | | | | | | | | | | | | | | PACSIN1, |
| | | | | | | | | | | | | | | | | HTR1A, |
| | | | | | | | | | | | | | | | | HTR1B, |
| | | | | | | | | | | | | | | | | INSR, |
| | | | | | | | | | | | | | | | | IRAK1, KDR, |
| | | | | | | | | | | | | | | | | KIT, LCK, |
| | | | | | | | | | | | | | | | | MARK3, |
| | | | | | | | | | | | | | | | | PIM3, |
| | | | | | | | | | | | | | | | | MAP3K9, |
| | | | | | | | | | | | | | | | | MAP3K11, |
| | | | | | | | | | | | | | | | | MYLK, |
| | | | | | | | | | | | | | | | | PIM1, |
| | | | | | | | | | | | | | | | | CSNK1G1, |
| | | | | | | | | | | | | | | | | AXL, |
| | | | | | | | | | | | | | | | | PRKCG, |
| | | | | | | | | | | | | | | | | PRKD1, |
| | | | | | | | | | | | | | | | | MAPK8, |
| | | | | | | | | | | | | | | | | CLK4, |
| | | | | | | | | | | | | | | | | MAP4K2, |
| | | | | | | | | | | | | | | | | RET, |
| | | | | | | | | | | | | | | | | RPS6KA2, |
| | | | | | | | | | | | | | | | | NUCKS1, |
| | | | | | | | | | | | | | | | | STK4, |
| | | | | | | | | | | | | | | | | STK10, |
| | | | | | | | | | | | | | | | | AURKC, |
| | | | | | | | | | | | | | | | | MAP3K7, |
| | | | | | | | | | | | | | | | | TGFBR1, |
| | | | | | | | | | | | | | | | | YES1, |
| | | | | | | | | | | | | | | | | CAMK2D, |
| | | | | | | | | | | | | | | | | ULK1, |
| | | | | | | | | | | | | | | | | DYRK3, |
| | | | | | | | | | | | | | | | | DYRK2, |
| | | | | | | | | | | | | | | | | MAP4K3, |
| | | | | | | | | | | | | | | | | RIPK2, |
| | | | | | | | | | | | | | | | | ACVR1B, |
| | | | | | | | | | | | | | | | | AURKB, |
| | | | | | | | | | | | | | | | | STK17A, |
| | | | | | | | | | | | | | | | | IKBKE, |
| | | | | | | | | | | | | | | | | ULK2, |
| | | | | | | | | | | | | | | | | MELK, |
| | | | | | | | | | | | | | | | | NUAK1 |
| 81 | 446.4 | C19 H17 | 2.0 | 10 | 0 | 93.9 | 9 | −24 | −8 | 43 | 43 | 75 | 124 | −40 | 124 | GPR119 |
| | | F3 N8 O2 | | | | | | | | | | | | | | |
| 82 | 394.5 | C22 H26 | 5.4 | 7 | 1 | 69.6 | 6 | 7 | 45 | 130 | 130 | 208 | 157 | 187 | 208 | LOC159691 |
| | | N4 O3 | | | | | | | | | | | | | | |
| 83 | 287.3 | C17 H13 | 2.3 | 5 | 1 | 57.9 | 2 | 52 | 100 | 183 | 183 | −42 | 3 | 1 | 3 | TNK2, |
| | | N5 | | | | | | | | | | | | | | IRAK3, KIT, |
| | | | | | | | | | | | | | | | | TGFBR1 |
| 84 | 410.5 | C22 H34 | 1.3 | 7 | 3 | 121.8 | 4 | −2 | 72 | 307 | 307 | 19 | 16 | −36 | 19 | |
| | | O7 | | | | | | | | | | | | | | |
| 85 | 351.4 | C22 H25 | 6.4 | 4 | 2 | 73.4 | 4 | 38 | 66 | 51 | 66 | 15 | 53 | −7 | 53 | RARA, |
| | | N O3 | | | | | | | | | | | | | | RARB, |
| | | | | | | | | | | | | | | | | RARG |
| 86 | 457 | C23 H25 | 4.8 | 6 | 0 | 60.3 | 4 | | | | | | | | | ESR1, |
| | | Cl N4 | | | | | | | | | | | | | | BRD4, |
| | | O2 S | | | | | | | | | | | | | | NR3C1, |
| | | | | | | | | | | | | | | | | PGR, BRD2, |
| | | | | | | | | | | | | | | | | BRDT, |
| | | | | | | | | | | | | | | | | BRD3 |
| 87 | 429.9 | C20 H16 | 1.6 | 7 | 2 | 104.6 | 5 | | | | | 17 | −22 | −14 | 17 | MAPK14, |
| | | Cl N3 | | | | | | | | | | | | | | MAPK1, |
| | | O4 S | | | | | | | | | | | | | | MAPK11, |
| | | | | | | | | | | | | | | | | MAP2K6, |
| | | | | | | | | | | | | | | | | STK10, |
| | | | | | | | | | | | | | | | | MAPKAPK3, |
| | | | | | | | | | | | | | | | | MAPKAPK2 |
| 88 | 234.3 | C11 H10 | 2.2 | 4 | 1 | 56.2 | 3 | 16 | 36 | 51 | 51 | −1 | −29 | 108 | 108 | |
| | | N2 O2 S | | | | | | | | | | | | | | |
| 89 | 415.5 | C27 H29 | 5.9 | 4 | 1 | 59.9 | 5 | 5 | 25 | 169 | 169 | −21 | 3 | 40 | 40 | |
| | | N O3 | | | | | | | | | | | | | | |
| 90 | 415.5 | C27 H29 | 5.9 | 4 | 1 | 59.9 | 5 | 7 | 0 | 144 | 144 | −2 | 6 | −47 | 6 | |
| | | N O3 | | | | | | | | | | | | | | |
| 91 | 292.4 | C14 H16 | 1.8 | 5 | 2 | 78.7 | 6 | 26 | 61 | 155 | 155 | 38 | 74 | 159 | 159 | |
| | | N2 O3 S | | | | | | | | | | | | | | |
| 92 | 415.5 | C23 H21 | 2.3 | 8 | 1 | 85.7 | 4 | | | | | −66 | −96 | −98 | −66 | ESR1, |
| | | N5 O3 | | | | | | | | | | | | | | BRD4, PGR, |
| | | | | | | | | | | | | | | | | SSTR4 |
| 93 | 366.4 | C24 H18 | 4.6 | 4 | 2 | 64.8 | 4 | −1 | −4 | 142 | 142 | | | | | |
| | | N2 O2 | | | | | | | | | | | | | | |
| 94 | 379.5 | C25 H21 | 6.3 | 4 | 1 | 46.0 | 5 | −6 | 6 | 254 | 254 | 33 | 32 | 16 | 33 | |
| | | N3 O | | | | | | | | | | | | | | |
| 95 | 376.9 | C18 H17 | 3.9 | 5 | 1 | 75.1 | 3 | 0 | 20 | 128 | 128 | −13 | 24 | 1 | 24 | |
| | | Cl N2 | | | | | | | | | | | | | | |
| | | O3 S | | | | | | | | | | | | | | |
| 96 | 319.4 | C15 H17 | 4.4 | 6 | 1 | 86.9 | 7 | 20 | 51 | 52 | 52 | 160 | 80 | 113 | 160 | |
| | | N3 O3 S | | | | | | | | | | | | | | |
| 97 | 372.4 | C19 H20 | 4.0 | 6 | 1 | 70.1 | 7 | −1 | 8 | 65 | 65 | 125 | 128 | 126 | 128 | |
| | | N2 O4 S | | | | | | | | | | | | | | |
| 98 | 681.6 | C35 H29 | 8.6 | 8 | 1 | 77.4 | 8 | 33 | 146 | 234 | 234 | −60 | −32 | −56 | −32 | |
| | | F6 N5 | | | | | | | | | | | | | | |
| | | O3 | | | | | | | | | | | | | | |
| 99 | 568.6 | C29 H31 | 4.7 | 9 | 2 | 97.3 | 12 | 17 | 156 | −57 | 156 | 43 | 242 | −53 | 242 | CDK5, |
| | | F3 N6 | | | | | | | | | | | | | | CDK8, |
| | | O3 | | | | | | | | | | | | | | RIPK3, |
| | | | | | | | | | | | | | | | | MAP4K1, |
| | | | | | | | | | | | | | | | | MAPK14, |
| | | | | | | | | | | | | | | | | CSF1R, |
| | | | | | | | | | | | | | | | | HIPK4, |
| | | | | | | | | | | | | | | | | EGFR, AHR, |
| | | | | | | | | | | | | | | | | EPHA2, |
| | | | | | | | | | | | | | | | | EPHA3, |
| | | | | | | | | | | | | | | | | EPHA4, |
| | | | | | | | | | | | | | | | | EPHA5, |
| | | | | | | | | | | | | | | | | EPHA7, |
| | | | | | | | | | | | | | | | | EPHA8, |
| | | | | | | | | | | | | | | | | EPHB1, |
| | | | | | | | | | | | | | | | | EPHB2, |
| | | | | | | | | | | | | | | | | MAPK15, |
| | | | | | | | | | | | | | | | | FGFR1, |
| | | | | | | | | | | | | | | | | FGFR2, |
| | | | | | | | | | | | | | | | | FGR, TNIK, |
| | | | | | | | | | | | | | | | | CDK19, |
| | | | | | | | | | | | | | | | | FLT1, FLT3, |
| | | | | | | | | | | | | | | | | FLT4, FRK, |
| | | | | | | | | | | | | | | | | ABL1, FYN, |
| | | | | | | | | | | | | | | | | ABL2, |
| | | | | | | | | | | | | | | | | STK39, |
| | | | | | | | | | | | | | | | | EPHA6, |
| | | | | | | | | | | | | | | | | MKNK2, |
| | | | | | | | | | | | | | | | | GSK3B, |
| | | | | | | | | | | | | | | | | HCK, |
| | | | | | | | | | | | | | | | | HTR1B, |
| | | | | | | | | | | | | | | | | HTR1D, |
| | | | | | | | | | | | | | | | | JAK1, JAK2, |
| | | | | | | | | | | | | | | | | KDR, KIT, |
| | | | | | | | | | | | | | | | | LCK, LYN, |
| | | | | | | | | | | | | | | | | MUSK, |
| | | | | | | | | | | | | | | | | NTRK1, |
| | | | | | | | | | | | | | | | | NTRK2, |
| | | | | | | | | | | | | | | | | NTRK3, |
| | | | | | | | | | | | | | | | | DDR2, |
| | | | | | | | | | | | | | | | | MINK1, |
| | | | | | | | | | | | | | | | | NIN, |
| | | | | | | | | | | | | | | | | CDK16, |
| | | | | | | | | | | | | | | | | PDGFRB, |
| | | | | | | | | | | | | | | | | ZAK, |
| | | | | | | | | | | | | | | | | CDK14, |
| | | | | | | | | | | | | | | | | PRKACG, |
| | | | | | | | | | | | | | | | | MAPK11, |
| | | | | | | | | | | | | | | | | MAP2K6, |
| | | | | | | | | | | | | | | | | MAP4K2, |
| | | | | | | | | | | | | | | | | RET, |
| | | | | | | | | | | | | | | | | MAPK12, |
| | | | | | | | | | | | | | | | | BLK, SRC, |
| | | | | | | | | | | | | | | | | BRAF, |
| | | | | | | | | | | | | | | | | MAP3K7, |
| | | | | | | | | | | | | | | | | TEK, YES1, |
| | | | | | | | | | | | | | | | | DDR1, |
| | | | | | | | | | | | | | | | | FIP1L1, |
| | | | | | | | | | | | | | | | | RIPK2, |
| | | | | | | | | | | | | | | | | CCNC, |
| | | | | | | | | | | | | | | | | MAP4K4 |
| 100 | 569.6 | C28 H30 | 5.1 | 10 | 3 | 111.6 | 11 | 59 | −58 | −67 | 59 | 245 | −99 | −100 | 245 | MERTK, |
| | | F3 N7 O3 | | | | | | | | | | | | | | EGFR, FLT3, |
| | | | | | | | | | | | | | | | | KDR, KIT, |
| | | | | | | | | | | | | | | | | AXL, |
| | | | | | | | | | | | | | | | | MAP2K1 |
| 101 | 449.5 | C23 H23 | 2.7 | 8 | 3 | 119.7 | 4 | | | | | 1 | 188 | 202 | 202 | CDK2, |
| | | N5 O3 S | | | | | | | | | | | | | | CDK3, |
| | | | | | | | | | | | | | | | | CCNO, |
| | | | | | | | | | | | | | | | | MERTK, |
| | | | | | | | | | | | | | | | | MAP4K5, |
| | | | | | | | | | | | | | | | | MAP4K1, |
| | | | | | | | | | | | | | | | | LRRK2, |
| | | | | | | | | | | | | | | | | DYRK1A, |
| | | | | | | | | | | | | | | | | EGFR, |
| | | | | | | | | | | | | | | | | MAPK15, |
| | | | | | | | | | | | | | | | | TNIK, FLT3, |
| | | | | | | | | | | | | | | | | GSK3A, |
| | | | | | | | | | | | | | | | | GSK3B, |
| | | | | | | | | | | | | | | | | IRAK1, |
| | | | | | | | | | | | | | | | | LCK, |
| | | | | | | | | | | | | | | | | MINK1, |
| | | | | | | | | | | | | | | | | PIK3CA, |
| | | | | | | | | | | | | | | | | PIK3CD, |
| | | | | | | | | | | | | | | | | PIK3CG, |
| | | | | | | | | | | | | | | | | CSNK1G1, |
| | | | | | | | | | | | | | | | | MAPK1, |
| | | | | | | | | | | | | | | | | CCNB1IP1, |
| | | | | | | | | | | | | | | | | BMPR1A, |
| | | | | | | | | | | | | | | | | BMPR2, |
| | | | | | | | | | | | | | | | | STK3, STK4, |
| | | | | | | | | | | | | | | | | MAP3K7, |
| | | | | | | | | | | | | | | | | TGFBR1, |
| | | | | | | | | | | | | | | | | MAP4K3, |
| | | | | | | | | | | | | | | | | CCNB3, |
| | | | | | | | | | | | | | | | | RIPK2, |
| | | | | | | | | | | | | | | | | CCNA2, |
| | | | | | | | | | | | | | | | | CCNB1, |
| | | | | | | | | | | | | | | | | CCNE1, |
| | | | | | | | | | | | | | | | | ACVR1, |
| | | | | | | | | | | | | | | | | MAP3K14, |
| | | | | | | | | | | | | | | | | CCNE2, |
| | | | | | | | | | | | | | | | | ACVR2A, |
| | | | | | | | | | | | | | | | | STK17B, |
| | | | | | | | | | | | | | | | | ACVR2B, |
| | | | | | | | | | | | | | | | | ACVRL1, |
| | | | | | | | | | | | | | | | | MAP4K4, |
| | | | | | | | | | | | | | | | | CDK1, |
| | | | | | | | | | | | | | | | | NUAK1 |
| 102 | 262.4 | C15 H22 | 2.6 | 4 | 2 | 56.3 | 4 | 3 | 30 | 54 | 54 | 83 | 237 | 18 | 237 | |
| | | N2 O2 | | | | | | | | | | | | | | |
| 103 | 388.5 | C23 H24 | 3.4 | 6 | 3 | 89.8 | 4 | −4 | −4 | 88 | 88 | 47 | 21 | 203 | 203 | MAP4K1, |
| | | N4 O2 | | | | | | | | | | | | | | LCK, |
| | | | | | | | | | | | | | | | | PIK3CD, |
| | | | | | | | | | | | | | | | | PIK3CG, |
| | | | | | | | | | | | | | | | | MAP4K3 |
| 104 | 537.6 | C29 H30 | 6.5 | 7 | 2 | 68.5 | 10 | | | | | 35 | 8 | 22 | 35 | MAP4K1, |
| | | F3 N5 O2 | | | | | | | | | | | | | | LCK, |
| | | | | | | | | | | | | | | | | MAP4K2 |
| 105 | 642.8 | C32 H42 | 5.4 | 12 | 1 | 118.2 | 15 | | | | | 36 | −22 | 75 | 75 | |
| | | N4 O8 S | | | | | | | | | | | | | | |

TABLE 2

| | | | Princ. | Princ. | Dist |
|---|---|---|---|---|---|
| | | Split | Result | Result | Act |
| Test Short Name | Split | Value | Type | Value | Flag |
| LRRK2 Hu Phos FRET-Adapta SP | Test Conc(μM):ATP | 1:75 | Mean Mean | 13.2 | N |
| | Conc(μM) | | Inhib (%) | | |
| PI4KB FRET-Adapta SP | Test Conc(μM):ATP | 1:5 | Mean Mean | −9.0 | N |
| | Conc(μM) | | Inhib (%) | | |
| PIK3C2A FRET-Adapta SP | Test Conc(μM):ATP | 1:25 | Mean Mean | 1.7 | N |
| | Conc(μM) | | Inhib (%) | | |
| PIK3C2B FRET-Adapta SP | Test Conc(μM):ATP | 1:10 | Mean Mean | 3.7 | N |
| | Conc(μM) | | Inhib (%) | | |
| PIK3C3 (hVPS34) *Mn* Adapta SP | Test Conc(μM):ATP | 1:10 | Mean Mean | 3.5 | N |
| | Conc(μM) | | Inhib (%) | | |
| PIK3CA/PIK3R1 FRET-Adapta SP | Test Conc(μM):ATP | 1:25 | Mean Mean | 8.4 | N |
| | Conc(μM) | | Inhib (%) | | |
| CAMK1 (CaMK1) FRET-Adapta SP | Test Conc(μM):ATP | 1:10 | Mean Mean | −9.9 | N |
| | Conc(μM) | | Inhib (%) | | |
| CDK7/cyclin H/MNAT1 Adapta SP | Test Conc(μM):ATP | 1:150 | Mean Mean | −2.3 | N |
| | Conc(μM) | | Inhib (%) | | |
| CDK9/cyclin T1 FRET-Adapta SP | Test Conc(μM):ATP | 1:25 | Mean Mean | 17.2 | N |
| | Conc(μM) | | Inhib (%) | | |
| CHUK(IKK alpha) FRET-Adapta SP | Test Conc(μM):ATP | 1:10 | Mean Mean | −6.9 | N |
| | Conc(μM) | | Inhib (%) | | |
| DAPK1 FRET-Adapta SP | Test Conc(μM):ATP | 1:5 | Mean Mean | 1.4 | N |
| | Conc(μM) | | Inhib (%) | | |
| GSG2 (Haspin) FRET-Adapta SP | Test Conc(μM):ATP | 1:25 | Mean Mean | −5.6 | N |
| | Conc(μM) | | Inhib (%) | | |
| IRAK1 FRET-Adapta SP | Test Conc(μM):ATP | 1:25 | Mean Mean | 11.2 | N |
| | Conc(μM) | | Inhib (%) | | |
| NUAK1 (ARK5) FRET-Adapta SP | Test Conc(μM):ATP | 1:25 | Mean Mean | −5.6 | N |
| | Conc(μM) | | Inhib (%) | | |
| PI4KA FRET-Adapta SP | Test Conc(μM):ATP | 1:10 | Mean Mean | 4.3 | N |
| | Conc(μM) | | Inhib (%) | | |
| PIK3CB FRET-Adapta SP | Test Conc(μM):ATP | 1:150 | Mean Mean | −5.2 | N |
| | Conc(μM) | | Inhib (%) | | |
| PIK3CD/PIK3R1 FRET-Adapta SP | Test Conc(μM):ATP | 1:75 | Mean Mean | 1.0 | N |
| | Conc(μM) | | Inhib (%) | | |
| SPHK1 FRET-Adapta SP | Test Conc(μM):ATP | 1:10 | Mean Mean | 8.0 | N |
| | Conc(μM) | | Inhib (%) | | |
| SPHK2 FRET-Adapta SP | Test Conc(μM):ATP | 1:10 | Mean Mean | −9.4 | N |
| | Conc(μM) | | Inhib (%) | | |
| PIK3CG (p110 gamma) Adapta SP | Test Conc(μM):ATP | 1:25 | Mean Mean | 7.4 | N |
| | Conc(μM) | | Inhib (%) | | |
| DDR1 FRET-Lantha SP | Test Conc(μM) | 1 | Mean Mean | 82.4 | A |
| | | | Inhib (%) | | |
| EIF2AK2 (PKR) FRET-Lantha SP | Test Conc(μM) | 1 | Mean Mean | 2.8 | N |
| | | | Inhib (%) | | |
| EPHA3 FRET-Lantha SP | Test Conc(μM) | 1 | Mean Mean | 4.8 | N |
| | | | Inhib (%) | | |
| EPHA6 FRET-Lantha SP | Test Conc(μM) | 1 | Mean Mean | 1.9 | N |
| | | | Inhib (%) | | |
| DMPK FRET-Lantha SP | Test Conc(μM) | 1 | Mean Mean | 7.1 | N |
| | | | Inhib (%) | | |
| DYRK2 FRET-Lantha SP | Test Conc(μM) | 1 | Mean Mean | −0.5 | N |
| | | | Inhib (%) | | |
| CDK5 (Inactive) FRET-Lantha SP | Test Conc(μM) | 1 | Mean Mean | 5.2 | N |
| | | | Inhib (%) | | |
| CDK8/cyclin C FRET-Lantha SP | Test Conc(μM) | 1 | Mean Mean | 8.6 | N |
| | | | Inhib (%) | | |
| CDK9 (Inactive) FRET-Lantha SP | Test Conc(μM) | 1 | Mean Mean | 6.2 | N |
| | | | Inhib (%) | | |
| CDC7/DBF4 FRET-Lantha SP | Test Conc(μM) | 1 | Mean Mean | 4.9 | N |
| | | | Inhib (%) | | |
| CDK16(PCTK1)/cyclinY Lantha SP | Test Conc(μM) | 1 | Mean Mean | 3.4 | N |
| | | | Inhib (%) | | |
| CAMK2G FRET-Lantha SP | Test Conc(μM) | 1 | Mean Mean | −1.7 | N |
| | | | Inhib (%) | | |
| CAMKK1 (Alpha) FRET-Lantha SP | Test Conc(μM) | 1 | Mean Mean | −7.3 | N |
| | | | Inhib (%) | | |
| ACVR2A FRET-Lantha SP | Test Conc(μM) | 1 | Mean Mean | 3.8 | N |
| | | | Inhib (%) | | |
| ACVR2B FRET-Lantha SP | Test Conc(μM) | 1 | Mean Mean | −2.1 | N |
| | | | Inhib (%) | | |
| BMPR1A (ALK3) FRET-Lantha SP | Test Conc(μM) | 1 | Mean Mean | −1.5 | N |
| | | | Inhib (%) | | |
| BMPR1B (ALK6) FRET-Lantha SP | Test Conc(μM) | 1 | Mean Mean | 11.9 | N |
| | | | Inhib (%) | | |
| CAMKK2 (Beta) FRET-Lantha SP | Test Conc(μM) | 1 | Mean Mean | −2.5 | N |
| | | | Inhib (%) | | |
| CASK FRET-Lantha SP | Test Conc(μM) | 1 | Mean Mean | 16.8 | N |
| | | | Inhib (%) | | |
| DAPK2 FRET-Lantha SP | Test Conc(μM) | 1 | Mean Mean | −2.5 | N |
| | | | Inhib (%) | | |
| BMPR2 FRET-Lantha SP | Test Conc(μM) | 1 | Mean Mean | −4.1 | N |
| | | | Inhib (%) | | |
| BRSK2 FRET-Lantha SP | Test Conc(μM) | 1 | Mean Mean | 3.2 | N |
| | | | Inhib (%) | | |
| MAP2K6 (MKK6) FRET-Lantha SP | Test Conc(μM) | 1 | Mean Mean | −4.9 | N |
| | | | Inhib (%) | | |
| LIMK2 FRET-Lantha SP | Test Conc(μM) | 1 | Mean Mean | 0.5 | N |
| | | | Inhib (%) | | |
| MAP2K1 (MEK1) FRET-Lantha SP | Test Conc(μM) | 1 | Mean Mean | −3.2 | N |
| | | | Inhib (%) | | |
| GRK1 FRET-Lantha SP | Test Conc(μM) | 1 | Mean Mean | −3.1 | N |
| | | | Inhib (%) | | |
| ICK FRET-Lantha SP | Test Conc(μM) | 1 | Mean Mean | 0.3 | N |
| | | | Inhib (%) | | |
| EPHA7 FRET-Lantha SP | Test Conc(μM) | 1 | Mean Mean | 6.5 | N |
| | | | Inhib (%) | | |
| LATS2 FRET-Lantha SP | Test Conc(μM) | 1 | Mean Mean | 0.5 | N |
| | | | Inhib (%) | | |
| LIMK1 FRET-Lantha SP | Test Conc(μM) | 1 | Mean Mean | 2.2 | N |
| | | | Inhib (%) | | |
| MAP2K2 (MEK2) FRET-Lantha SP | Test Conc(μM) | 1 | Mean Mean | 1.7 | N |
| | | | Inhib (%) | | |
| LATS1 FRET-Lantha SP | Test Conc(μM) | 1 | Mean Mean | 19.8 | N |
| | | | Inhib (%) | | |
| MAPK10 (JNK3) FRET-Lantha SP | Test Conc(μM) | 1 | Mean Mean | −0.2 | N |
| | | | Inhib (%) | | |
| MAPK15 (ERK7) FRET-Lantha SP | Test Conc(μM) | 1 | Mean Mean | 0.8 | N |
| | | | Inhib (%) | | |
| MAP3K2 (MEKK2) FRET-Lantha SP | Test Conc(μM) | 1 | Mean Mean | 3.5 | N |
| | | | Inhib (%) | | |
| MAP3K3 (MEKK3) FRET-Lantha SP | Test Conc(μM) | 1 | Mean Mean | 5.2 | N |
| | | | Inhib (%) | | |
| MAP3K5 (ASK1) FRET-Lantha SP | Test Conc(μM) | 1 | Mean Mean | 0.2 | N |
| | | | Inhib (%) | | |
| MAP3K11 (MLK3) FRET-Lantha SP | Test Conc(μM) | 1 | Mean Mean | −0.7 | N |
| | | | Inhib (%) | | |
| MAP3K14 (NIK) FRET-Lantha SP | Test Conc(μM) | 1 | Mean Mean | −1.3 | N |
| | | | Inhib (%) | | |
| MAP3K10 (MLK2) FRET-Lantha SP | Test Conc(μM) | 1 | Mean Mean | 0.6 | N |
| | | | Inhib (%) | | |
| MAP4K1 (HPK1) FRET-Lantha SP | Test Conc(μM) | 1 | Mean Mean | −1.6 | N |
| | | | Inhib (%) | | |
| MAPK8 (JNK1) FRET-Lantha SP | Test Conc(μM) | 1 | Mean Mean | −6.1 | N |
| | | | Inhib (%) | | |
| MAPK9 (JNK2) FRET-Lantha SP | Test Conc(μM) | 1 | Mean Mean | 5.3 | N |
| | | | Inhib (%) | | |
| STK16 (PKL12) FRET-Lantha SP | Test Conc(μM) | 1 | Mean Mean | −1.7 | N |
| | | | Inhib (%) | | |
| PRKACG FRET-Lantha SP | Test Conc(μM) | 1 | Mean Mean | 0.7 | N |
| | | | Inhib (%) | | |
| MYLK (MLCK) FRET-Lantha SP | Test Conc(μM) | 1 | Mean Mean | 2.2 | N |
| | | | Inhib (%) | | |
| MYO3B FRET-Lantha SP | Test Conc(μM) | 1 | Mean Mean | 3.9 | N |
| | | | Inhib (%) | | |
| NLK FRET-Lantha SP | Test Conc(μM) | 1 | Mean Mean | −3.5 | N |
| | | | Inhib (%) | | |
| NUAK2 FRET-Lantha SP | Test Conc(μM) | 1 | Mean Mean | 14.1 | N |
| | | | Inhib (%) | | |
| MKNK2 (MNK2) FRET-Lantha SP | Test Conc(μM) | 1 | Mean Mean | 2.2 | N |
| | | | Inhib (%) | | |
| MAP3K7/MAP3K7IP1 Lantha SP | Test Conc(μM) | 1 | Mean Mean | 3.6 | N |
| | | | Inhib (%) | | |
| PLK4 FRET-Lantha SP | Test Conc(μM) | 1 | Mean Mean | 0.2 | N |
| | | | Inhib (%) | | |
| PRKACB FRET-Lantha SP | Test Conc(μM) | 1 | Mean Mean | 2.3 | N |
| | | | Inhib (%) | | |
| RIPK2 FRET-Lantha SP | Test Conc(μM) | 1 | Mean Mean | 13.0 | N |
| | | | Inhib (%) | | |
| RIPK3 FRET-Lantha SP | Test Conc(μM) | 1 | Mean Mean | 20.9 | N |
| | | | Inhib (%) | | |
| SIK3 FRET-Lantha SP | Test Conc(μM) | 1 | Mean Mean | 5.6 | N |
| | | | Inhib (%) | | |
| PKN2 (PRK2) FRET-lantha SP | Test Conc(μM) | 1 | Mean Mean | −2.6 | N |
| | | | Inhib (%) | | |
| STK17A (DRAK1) FRET-Lantha SP | Test Conc(μM) | 1 | Mean Mean | 4.6 | N |
| | | | Inhib (%) | | |
| TNIK FRET-Lantha SP | Test Conc(μM) | 1 | Mean Mean | 4.1 | N |
| | | | Inhib (%) | | |
| STK32C (YANK3) FRET-Lantha SP | Test Conc(μM) | 1 | Mean Mean | −1.0 | N |
| | | | Inhib (%) | | |
| STK33 FRET-Lantha SP | Test Conc(μM) | 1 | Mean Mean | 1.7 | N |
| | | | Inhib (%) | | |
| STK38L (NDR2) FRET-Lantha SP | Test Conc(μM) | 1 | Mean Mean | 15.6 | N |
| | | | Inhib (%) | | |
| STK39 (STLK3) FRET-Lantha SP | Test Conc(μM) | 1 | Mean Mean | 6.0 | N |
| | | | Inhib (%) | | |
| STK32B (YANK2) FRET-Lantha SP | Test Conc(μM) | 1 | Mean Mean | −1.3 | N |
| | | | Inhib (%) | | |
| STK17B (DRAK2) FRET-Lantha SP | Test Conc(μM) | 1 | Mean Mean | 4.7 | N |
| | | | Inhib (%) | | |
| TAOK3 (JIK) FRET-Lantha SP | Test Conc(μM) | 1 | Mean Mean | 0.3 | N |
| | | | Inhib (%) | | |
| TEC FRET-Lantha SP | Test Conc(μM) | 1 | Mean Mean | 5.7 | N |
| | | | Inhib (%) | | |
| TGFBR2 FRET-Lantha SP | Test Conc(μM) | 1 | Mean Mean | −3.5 | N |
| | | | Inhib (%) | | |
| TLK1 FRET-Lantha SP | Test Conc(μM) | 1 | Mean Mean | −4.3 | N |
| | | | Inhib (%) | | |
| TLK2 FRET-Lantha SP | Test Conc(μM) | 1 | Mean Mean | −2.9 | N |
| | | | Inhib (%) | | |
| TNK2 (ACK) FRET-Lantha SP | Test Conc(μM) | 1 | Mean Mean | 0.2 | N |
| | | | Inhib (%) | | |
| TTK FRET-Lantha SP | Test Conc(μM) | 1 | Mean Mean | 6.5 | N |
| | | | Inhib (%) | | |
| ULK3 FRET-Lantha SP | Test Conc(μM) | 1 | Mean Mean | −2.1 | N |
| | | | Inhib (%) | | |
| WEE1 FRET-Lantha SP | Test Conc(μM) | 1 | Mean Mean | 4.6 | N |
| | | | Inhib (%) | | |
| ULK1 FRET-Lantha SP | Test Conc(μM) | 1 | Mean Mean | 1.6 | N |
| | | | Inhib (%) | | |
| ULK2 FRET-Lantha SP | Test Conc(μM) | 1 | Mean Mean | 2.6 | N |
| | | | Inhib (%) | | |
| TAOK1 FRET-Lantha SP | Test Conc(μM) | 1 | Mean Mean | −6.0 | N |
| | | | Inhib (%) | | |
| WNK2 FRET-Lantha SP | Test Conc(μM) | 1 | Mean Mean | 9.6 | N |
| | | | Inhib (%) | | |
| WNK3 FRET-Lantha SP | Test Conc(μM) | 1 | Mean Mean | 5.3 | N |
| | | | Inhib (%) | | |
| ZAK FRET-Lantha SP | Test Conc(μM) | 1 | Mean Mean | 3.1 | N |
| | | | Inhib (%) | | |
| ABL2 (Arg) FRET-Z-Lyte SP | Test Conc(μM):ATP | 1:25 | Mean Mean | −1.3 | N |
| | Conc(μM) | | Inhib (%) | | |
| ACVR1B (ALK4) *Mn* Z-Lyte SP | Test Conc(μM):ATP | 1:5 | Mean Mean | 6.8 | N |
| | Conc(μM) | | Inhib (%) | | |
| EPHB4 FRET-Z-Lyte SP | Test Conc(μM):ATP | 1:100 | Mean Mean | 1.4 | N |
| | Conc(μM) | | Inhib (%) | | |
| GRK6 FRET-Z-Lyte SP | Test Conc(μM):ATP | 1:10 | Mean Mean | 0.8 | N |
| | Conc(μM) | | Inhib (%) | | |
| EPHB3 FRET-Z-Lyte SP | Test Conc(μM):ATP | 1:75 | Mean Mean | −0.5 | N |
| | Conc(μM) | | Inhib (%) | | |
| GRK7 FRET-Z-Lyte SP | Test Conc(μM):ATP | 1:10 | Mean Mean | 4.6 | N |
| | Conc(μM) | | Inhib (%) | | |
| GSK3A FRET-Z-Lyte SP | Test Conc(μM):ATP | 1:10 | Mean Mean | 9.4 | N |
| | Conc(μM) | | Inhib (%) | | |
| GSK3B FRET-Z-Lyte SP | Test Conc(μM):ATP | 1:10 | Mean Mean | 6.5 | N |
| | Conc(μM) | | Inhib (%) | | |
| ITK FRET-Z-Lyte SP | Test Conc(μM):ATP | 1:5 | Mean Mean | 6.1 | N |
| | Conc(μM) | | Inhib (%) | | |
| JAK1 FRET-Z-Lyte SP | Test Conc(μM):ATP | 1:75 | Mean Mean | −1.9 | N |
| | Conc(μM) | | Inhib (%) | | |
| JAK2 FRET-Z-Lyte SP | Test Conc(μM):ATP | 1:25 | Mean Mean | −7.7 | N |
| | Conc(μM) | | Inhib (%) | | |
| JAK3 FRET-Z-Lyte SP | Test Conc(μM):ATP | 1:10 | Mean Mean | 7.2 | N |
| | Conc(μM) | | Inhib (%) | | |
| LCK FRET-Z-Lyte SP | Test Conc(μM):ATP | 1:50 | Mean Mean | −4.1 | N |
| | Conc(μM) | | Inhib (%) | | |
| HCK FRET-Z-Lyte SP | Test Conc(μM):ATP | 1:25 | Mean Mean | 6.4 | N |
| | Conc(μM) | | Inhib (%) | | |
| HIPK1 (Myak) FRET-Z-Lyte SP | Test Conc(μM):ATP | 1:5 | Mean Mean | 1.3 | N |
| | Conc(μM) | | Inhib (%) | | |
| KDR (VEGFR2) FRET-Z-Lyte SP | Test Conc(μM):ATP | 1:75 | Mean Mean | 3.2 | N |
| | Conc(μM) | | Inhib (%) | | |
| HIPK2 FRET-Z-Lyte SP | Test Conc(μM):ATP | 1:10 | Mean Mean | −3.2 | N |
| | Conc(μM) | | Inhib (%) | | |
| HIPK3 (YAK1) FRET-Z-Lyte SP | Test Conc(μM):ATP | 1:25 | Mean Mean | 0.6 | N |
| | Conc(μM) | | Inhib (%) | | |
| HIPK4 FRET-Z-Lyte SP | Test Conc(μM):ATP | 1:50 | Mean Mean | −2.8 | N |
| | Conc(μM) | | Inhib (%) | | |
| IGF1R FRET-Z-Lyte SP | Test Conc(μM):ATP | 1:150 | Mean Mean | 11.6 | N |
| | Conc(μM) | | Inhib (%) | | |
| IKBKB (IKKbeta) FRET-Z-Lyte SP | Test Conc(μM):ATP | 1:5 | Mean Mean | 0.6 | N |
| | Conc(μM) | | Inhib (%) | | |
| IKBKE (IKK epsilon) Z-Lyte SP | Test Conc(μM):ATP | 1:10 | Mean Mean | −3.5 | N |
| | Conc(μM) | | Inhib (%) | | |
| INSR *Mn* FRET-Z-Lyte SP | Test Conc(μM):ATP | 1:25 | Mean Mean | 2.1 | N |
| | Conc(μM) | | Inhib (%) | | |
| INSRR (IRR) *Mn* Z-Lyte SP | Test Conc(μM):ATP | 1:50 | Mean Mean | 13.5 | N |
| | Conc(μM) | | Inhib (%) | | |
| IRAK4 *Mn* FRET-Z-Lyte SP | Test Conc(μM):ATP | 1:25 | Mean Mean | 12.7 | N |
| | Conc(μM) | | Inhib (%) | | |
| CLK2 FRET-Z-Lyte SP | Test Conc(μM):ATP | 1:25 | Mean Mean | 3.7 | N |
| | Conc(μM) | | Inhib (%) | | |
| CLK3 FRET-Z-Lyte SP | Test Conc(μM):ATP | 1:150 | Mean Mean | 2.0 | N |
| | Conc(μM) | | Inhib (%) | | |
| CSF1R (FMS) FRET-Z-Lyte SP | Test Conc(μM):ATP | 1:500 | Mean Mean | 12.1 | N |
| | Conc(μM) | | Inhib (%) | | |
| CSK FRET-Z-Lyte SP | Test Conc(μM):ATP | 1:10 | Mean Mean | 10.1 | N |
| | Conc(μM) | | Inhib (%) | | |
| CSNK1A1 FRET-Z-Lyte SP | Test Conc(μM):ATP | 1:5 | Mean Mean | 4.2 | N |
| | Conc(μM) | | Inhib (%) | | |
| ERBB2 (HER2) *Mn* Z-Lyte SP | Test Conc(μM):ATP | 1:10 | Mean Mean | 2.7 | N |
| | Conc(μM) | | Inhib (%) | | |
| CAMK1D FRET-Z-Lyte SP | Test Conc(μM):ATP | 1:25 | Mean Mean | 0.0 | N |
| | Conc(μM) | | Inhib (%) | | |
| CAMK2A FRET-Z-Lyte SP | Test Conc(μM):ATP | 1:10 | Mean Mean | 1.0 | N |
| | Conc(μM) | | Inhib (%) | | |
| CAMK2B FRET-Z-Lyte SP | Test Conc(μM):ATP | 1:75 | Mean Mean | 4.5 | N |
| | Conc(μM) | | Inhib (%) | | |
| ADRBK1 (GRK2) FRET-Z-Lyte SP | Test Conc(μM):ATP | 1:10 | Mean Mean | 8.4 | N |
| | Conc(μM) | | Inhib (%) | | |
| ADRBK2 (GRK3) FRET-Z-Lyte SP | Test Conc(μM):ATP | 1:10 | Mean Mean | 0.7 | N |
| | Conc(μM) | | Inhib (%) | | |
| AKT1 (PKB alpha) Z-Lyte SP | Test Conc(μM):ATP | 1:75 | Mean Mean | 5.2 | N |
| | Conc(μM) | | Inhib (%) | | |
| AKT2 (PKB beta) FRET-Z-Lyte SP | Test Conc(μM):ATP | 1:200 | Mean Mean | 8.0 | N |
| | Conc(μM) | | Inhib (%) | | |
| AKT3 (PKB gamma) Z-Lyte SP | Test Conc(μM):ATP | 1:100 | Mean Mean | 5.8 | N |
| | Conc(μM) | | Inhib (%) | | |
| ALK FRET-Z-Lyte SP | Test Conc(μM):ATP | 1:25 | Mean Mean | 11.9 | N |
| | Conc(μM) | | Inhib (%) | | |
| AURKA (Aurora A) Z-Lyte SP | Test Conc(μM):ATP | 1:10 | Mean Mean | −2.7 | N |
| | Conc(μM) | | Inhib (%) | | |
| AURKB (Aurora B) Z-Lyte SP | Test Conc(μM):ATP | 1:75 | Mean Mean | 4.9 | N |
| | Conc(μM) | | Inhib (%) | | |
| AURKC (Aurora C) Z-Lyte SP | Test Conc(μM):ATP | 1:25 | Mean Mean | 9.3 | N |
| | Conc(μM) | | Inhib (%) | | |
| AXL FRET-Z-Lyte SP | Test Conc(μM):ATP | 1:75 | Mean Mean | −2.7 | N |
| | Conc(μM) | | Inhib (%) | | |
| CAMK2D FRET-Z-Lyte SP | Test Conc(μM):ATP | 1:5 | Mean Mean | 8.1 | N |
| | Conc(μM) | | Inhib (%) | | |
| CAMK4 (CaMKIV) FRET-Z-Lyte SP | Test Conc(μM):ATP | 1:25 | Mean Mean | −5.4 | N |
| | Conc(μM) | | Inhib (%) | | |
| CDC42 BPA (MRCKA) Z-Lyte SP | Test Conc(μM):ATP | 1:5 | Mean Mean | −2.0 | N |
| | Conc(μM) | | Inhib (%) | | |
| CSNK1D (CK1 delta) Z-Lyte SP | Test Conc(μM):ATP | 1:5 | Mean Mean | 1.2 | N |
| | Conc(μM) | | Inhib (%) | | |
| CSNK1E (CK1 epsilon) Z-Lyte SP | Test Conc(μM):ATP | 1:5 | Mean Mean | 5.8 | N |
| | Conc(μM) | | Inhib (%) | | |
| CSNK1G1 (CK1gamma1) Z-Lyte SP | Test Conc(μM):ATP | 1:5 | Mean Mean | 5.2 | N |
| | Conc(μM) | | Inhib (%) | | |
| CSNK1G2 (CK1gamma2) Z-Lyte SP | Test Conc(μM):ATP | 1:5 | Mean Mean | 3.9 | N |
| | Conc(μM) | | Inhib (%) | | |
| DAPK3 (ZIPK) FRET-Z-Lyte SP | Test Conc(μM):ATP | 1:5 | Mean Mean | 3.3 | N |
| | Conc(μM) | | Inhib (%) | | |
| DCAMKL2 (DCK2) FRET-Z-Lyte SP | Test Conc(μM):ATP | 1:150 | Mean Mean | 5.5 | N |
| | Conc(μM) | | Inhib (%) | | |
| DNA-PK FRET-Z-Lyte SP | Test Conc(μM):ATP | 1:25 | Mean Mean | 13.7 | N |
| | Conc(μM) | | Inhib (%) | | |
| BRSK1 (SAD1) FRET-Z-Lyte SP | Test Conc(μM):ATP | 1:25 | Mean Mean | 2.2 | N |
| | Conc(μM) | | Inhib (%) | | |
| BTK FRET-Z-Lyte SP | Test Conc(μM):ATP | 1:25 | Mean Mean | 6.7 | N |
| | Conc(μM) | | Inhib (%) | | |
| BLK FRET-Z-Lyte SP | Test Conc(μM):ATP | 1:25 | Mean Mean | 3.0 | N |
| | Conc(μM) | | Inhib (%) | | |
| BMX FRET-Z-Lyte SP | Test Conc(μM):ATP | 1:100 | Mean Mean | 7.2 | N |
| | Conc(μM) | | Inhib (%) | | |
| DYRK1A FRET-Z-Lyte SP | Test Conc(μM):ATP | 1:100 | Mean Mean | 6.2 | N |
| | Conc(μM) | | Inhib (%) | | |
| DYRK1B FRET-Z-Lyte SP | Test Conc(μM):ATP | 1:75 | Mean Mean | 3.0 | N |
| | Conc(μM) | | Inhib (%) | | |
| DYRK3 FRET-Z-Lyte SP | ATP Conc(μM):Test | 1:5 | Mean Mean | 0.4 | N |
| | Conc(μM) | | Inhib (%) | | |
| CSNK1G3 (CK1gamma3) Z-Lyte SP | Test Conc(μM):ATP | 1:5 | Mean Mean | 2.9 | N |
| | Conc(μM) | | Inhib (%) | | |
| DYRK4 FRET-Z-Lyte SP | Test Conc(μM):ATP | 1:5 | Mean Mean | 0.9 | N |
| | Conc(μM) | | Inhib (%) | | |
| CSNK2A1 (CK2alpha1) Z-Lyte SP | Test Conc(μM):ATP | 1:5 | Mean Mean | 6.1 | N |
| | Conc(μM) | | Inhib (%) | | |
| CSNK2A2 (CK2alpha2) Z-Lyte SP | Test Conc(μM):ATP | 1:50 | Mean Mean | 9.9 | N |
| | Conc(μM) | | Inhib (%) | | |
| CDC42 BPB (MRCKB) Z-Lyte SP | Test Conc(μM):ATP | 1:5 | Mean Mean | −7.0 | N |
| | Conc(μM) | | Inhib (%) | | |
| CDK1/cyclin B FRET-Z-Lyte SP | Test Conc(μM):ATP | 1:25 | Mean Mean | 3.8 | N |
| | Conc(μM) | | Inhib (%) | | |
| CDK2/cyclin A FRET-Z-Lyte SP | Test Conc(μM):ATP | 1:25 | Mean Mean | 6.2 | N |
| | Conc(μM) | | Inhib (%) | | |
| CHEK1 (CHK1) FRET-Z-Lyte SP | Test Conc(μM):ATP | 1:50 | Mean Mean | 6.3 | N |
| | Conc(μM) | | Inhib (%) | | |
| CHEK2 (CHK2) FRET-Z-Lyte SP | Test Conc(μM):ATP | 1:75 | Mean Mean | 10.2 | N |
| | Conc(μM) | | Inhib (%) | | |
| FGFR2 *Mn* FRET-Z-Lyte SP | Test Conc(μM):ATP | 1:5 | Mean Mean | 15.8 | N |
| | Conc(μM) | | Inhib (%) | | |
| FGFR4 *Mn* FRET-Z-Lyte SP | Test Conc(μM):ATP | 1:150 | Mean Mean | 14.8 | N |
| | Conc(μM) | | Inhib (%) | | |
| FGR FRET-Z-Lyte SP | Test Conc(μM):ATP | 1:10 | Mean Mean | 9.4 | N |
| | Conc(μM) | | Inhib (%) | | |
| EPHA2 FRET-Z-Lyte SP | Test Conc(μM):ATP | 1:75 | Mean Mean | 10.8 | N |
| | Conc(μM) | | Inhib (%) | | |
| EPHA4 FRET-Z-Lyte SP | Test Conc(μM):ATP | 1:100 | Mean Mean | 6.9 | N |
| | Conc(μM) | | Inhib (%) | | |
| EPHA5 FRET-Z-Lyte SP | Test Conc(μM):ATP | 1:150 | Mean Mean | 6.7 | N |
| | Conc(μM) | | Inhib (%) | | |
| EPHA1 FRET-Z-Lyte SP | Test Conc(μM):ATP | 1:25 | Mean Mean | 6.7 | N |
| | Conc(μM) | | Inhib (%) | | |
| EPHB2 FRET-Z-Lyte SP | Test Conc(μM):ATP | 1:75 | Mean Mean | 6.1 | N |
| | Conc(μM) | | Inhib (%) | | |
| EPHA8 FRET-Z-Lyte SP | Test Conc(μM):ATP | 1:100 | Mean Mean | 8.6 | N |
| | Conc(μM) | | Inhib (%) | | |
| EPHB1 FRET-Z-Lyte SP | Test Conc(μM):ATP | 1:50 | Mean Mean | 3.5 | N |
| | Conc(μM) | | Inhib (%) | | |
| EEF2K FRET-Z-Lyte SP | Test Conc(μM):ATP | 1:10 | Mean Mean | 5.5 | N |
| | Conc(μM) | | Inhib (%) | | |
| ERBB4 (HER4) *Mn* Z-Lyte SP | Test Conc(μM):ATP | 1:5 | Mean Mean | 0.8 | N |
| | Conc(μM) | | Inhib (%) | | |
| FER FRET-Z-Lyte SP | Test Conc(μM):ATP | 1:25 | Mean Mean | 4.8 | N |
| | Conc(μM) | | Inhib (%) | | |
| FGFR1 *Mn* FRET-Z-Lyte SP | Test Conc(μM):ATP | 1:25 | Mean Mean | 6.5 | N |
| | Conc(μM) | | Inhib (%) | | |
| FRAP1 (mTOR) *Mn* Z-Lyte SP | Test Conc(μM):ATP | 1:10 | Mean Mean | 0.7 | N |
| | Conc(μM) | | Inhib (%) | | |
| FLT4 (VEGFR3) *Mn* Z-Lyte SP | Test Conc(μM):ATP | 1:5 | Mean Mean | 2.8 | N |
| | Conc(μM) | | Inhib (%) | | |
| FRK (PTK5) FRET-Z-Lyte SP | Test Conc(μM):ATP | 1:50 | Mean Mean | 5.7 | N |
| | Conc(μM) | | Inhib (%) | | |
| GRK4 FRET-Z-Lyte SP | Test Conc(μM):ATP | 1:10 | Mean Mean | 0.0 | N |
| | Conc(μM) | | Inhib (%) | | |
| GRK5 *Mn* FRET-Z-Lyte SP | Test Conc(μM):ATP | 1:5 | Mean Mean | 4.7 | N |
| | Conc(μM) | | Inhib (%) | | |
| FYN FRET-Z-Lyte SP | Test Conc(μM):ATP | 1:75 | Mean Mean | 5.1 | N |
| | Conc(μM) | | Inhib (%) | | |
| NEK1 FRET-Z-Lyte SP | ATP Conc(μM):Test | 1:100 | Mean Mean | 7.0 | N |
| | Conc(μM) | | Inhib (%) | | |
| NEK2 FRET-Z-Lyte SP | Test Conc(μM):ATP | 1:150 | Mean Mean | −0.8 | N |
| | Conc(μM) | | Inhib (%) | | |
| NEK4 FRET-Z-Lyte SP | Test Conc(μM):ATP | 1:50 | Mean Mean | 14.2 | N |
| | Conc(μM) | | Inhib (%) | | |
| NTRK2 (TRKB) *Mn* Z-Lyte SP | Test Conc(μM):ATP | 1:25 | Mean Mean | 1.9 | N |
| | Conc(μM) | | Inhib (%) | | |
| NTRK3 (TRKC) FRET-Z-Lyte SP | Test Conc(μM):ATP | 1:50 | Mean Mean | 3.5 | N |
| | Conc(μM) | | Inhib (%) | | |
| PAK1 FRET-Z-Lyte SP | Test Conc(μM):ATP | 1:50 | Mean Mean | −1.9 | N |
| | Conc(μM) | | Inhib (%) | | |
| PAK2 (PAK65) FRET-Z-Lyte SP | Test Conc(μM):ATP | 1:75 | Mean Mean | 14.5 | N |
| | Conc(μM) | | Inhib (%) | | |
| PAK3 FRET-Z-Lyte SP | Test Conc(μM):ATP | 1:100 | Mean Mean | −0.3 | N |
| | Conc(μM) | | Inhib (%) | | |
| PAK4 FRET-Z-Lyte SP | Test Conc(μM):ATP | 1:5 | Mean Mean | 0.9 | N |
| | Conc(μM) | | Inhib (%) | | |
| NTRK1 (TRKA) FRET-Z-Lyte SP | Test Conc(μM):ATP | 1:400 | Mean Mean | 30.7 | N |
| | Conc(μM) | | Inhib (%) | | |
| MARK2 FRET-Z-Lyte SP | Test Conc(μM):ATP | 1:10 | Mean Mean | 7.4 | N |
| | Conc(μM) | | Inhib (%) | | |
| MARK3 FRET-Z-Lyte SP | Test Conc(μM):ATP | 1:5 | Mean Mean | 8.9 | N |
| | Conc(μM) | | Inhib (%) | | |
| MARK4 FRET-Z-Lyte SP | Test Conc(μM):ATP | 1:10 | Mean Mean | 0.2 | N |
| | Conc(μM) | | Inhib (%) | | |
| MATK (HYL) FRET-Z-Lyte SP | Test Conc(μM):ATP | 1:300 | Mean Mean | 3.2 | N |
| | Conc(μM) | | Inhib (%) | | |
| MELK FRET-Z-Lyte SP | Test Conc(μM):ATP | 1:25 | Mean Mean | 3.6 | N |
| | Conc(μM) | | Inhib (%) | | |
| MERTK (cMER) *Mn* Z-Lyte SP | Test Conc(μM):ATP | 1:10 | Mean Mean | 13.1 | N |
| | Conc(μM) | | Inhib (%) | | |
| NEK6 FRET-Z-Lyte SP | Test Conc(μM):ATP | 1:100 | Mean Mean | −4.0 | N |
| | Conc(μM) | | Inhib (%) | | |
| NEK7 FRET-Z-Lyte SP | Test Conc(μM):ATP | 1:50 | Mean Mean | 7.4 | N |
| | Conc(μM) | | Inhib (%) | | |
| NEK9 *Mn* FRET-Z-Lyte SP | Test Conc(μM):ATP | 1:100 | Mean Mean | 1.8 | N |
| | Conc(μM) | | Inhib (%) | | |
| MAPK13 (p38 delta) Z-Lyte SP | Test Conc(μM):ATP | 1:10 | Mean Mean | 4.7 | N |
| | Conc(μM) | | Inhib (%) | | |
| MAPK3 (ERK1) FRET-Z-Lyte SP | Test Conc(μM):ATP | 1:50 | Mean Mean | 5.1 | N |
| | Conc(μM) | | Inhib (%) | | |
| MAPK8 (JNK1) Cascade Z-Lyte SP | Test Conc(μM):ATP | 1:100 | Mean Mean | 6.5 | N |
| | Conc(μM) | | Inhib (%) | | |
| MAPK9 (JNK2) Cascade Z-Lyte SP | Test Conc(μM):ATP | 1:100 | Mean Mean | 17.9 | N |
| | Conc(μM) | | Inhib (%) | | |
| LTK (TYK1) FRET-Z-Lyte SP | Test Conc(μM):ATP | 1:75 | Mean Mean | −6.4 | N |
| | Conc(μM) | | Inhib (%) | | |
| MAP2K1 (MEK1)Cascade Z-Lyte SP | Test Conc(μM):ATP | 1:100 | Mean Mean | 20.0 | N |
| | Conc(μM) | | Inhib (%) | | |
| MAP2K2 (MEK2)Cascade Z-Lyte SP | Test Conc(μM):ATP | 1:100 | Mean Mean | −1.0 | N |
| | Conc(μM) | | Inhib (%) | | |
| MAP2K6 (MKK6)Cascade Z-Lyte SP | Test Conc(μM):ATP | 1:100 | Mean Mean | 21.3 | N |
| | Conc(μM) | | Inhib (%) | | |
| MAP3K8 (COT) Cascade Z-Lyte SP | Test Conc(μM):ATP | 1:100 | Mean Mean | 6.1 | N |
| | Conc(μM) | | Inhib (%) | | |
| MAP3K9 (MLK1) FRET-Z-Lyte SP | Test Conc(μM):ATP | 1:75 | Mean Mean | 1.3 | N |
| | Conc(μM) | | Inhib (%) | | |
| MAP4K2 (GCK) FRET-Z-Lyte SP | Test Conc(μM):ATP | 1:100 | Mean Mean | 13.1 | N |
| | Conc(μM) | | Inhib (%) | | |
| MAP4K4 (HGK) FRET-Z-Lyte SP | Test Conc(μM):ATP | 1:10 | Mean Mean | 16.9 | N |
| | Conc(μM) | | Inhib (%) | | |
| MAP4K5 (KHS1) FRET-Z-Lyte SP | Test Conc(μM):ATP | 1:50 | Mean Mean | −7.8 | N |
| | Conc(μM) | | Inhib (%) | | |
| MAPK1 (ERK2) FRET-Z-Lyte SP | Test Conc(μM):ATP | 1:100 | Mean Mean | 3.6 | N |
| | Conc(μM) | | Inhib (%) | | |
| MAPKAPK2 FRET-Z-Lyte SP | Test Conc(μM):ATP | 1:5 | Mean Mean | −2.1 | N |
| | Conc(μM) | | Inhib (%) | | |
| MAPKAPK3 FRET-Z-Lyte SP | Test Conc(μM):ATP | 1:200 | Mean Mean | 1.7 | N |
| | Conc(μM) | | Inhib (%) | | |
| MAPKAPK5 (PRAK) FRET-Z-Lyte SP | Test Conc(μM):ATP | 1:10 | Mean Mean | −1.3 | N |
| | Conc(μM) | | Inhib (%) | | |
| MARK1 (MARK) FRET-Z-Lyte SP | Test Conc(μM):ATP | 1:5 | Mean Mean | 16.9 | N |
| | Conc(μM) | | Inhib (%) | | |
| MAPK11 (p38 beta) Z-Lyte SP | Test Conc(μM):ATP | 1:50 | Mean Mean | 47.6 | N |
| | Conc(μM) | | Inhib (%) | | |
| MAPK12 (p38 gamma) Z-Lyte SP | Test Conc(μM):ATP | 1:10 | Mean Mean | 5.1 | N |
| | Conc(μM) | | Inhib (%) | | |
| MAPK10 (JNK3)Cascade Z-Lyte SP | Test Conc(μM):ATP | 1:100 | Mean Mean | 10.6 | N |
| | Conc(μM) | | Inhib (%) | | |
| SYK FRET-Z-Lyte SP | Test Conc(μM):ATP | 1:25 | Mean Mean | −3.9 | N |
| | Conc(μM) | | Inhib (%) | | |
| TAOK2 (TAO1) FRET-Z-Lyte SP | Test Conc(μM):ATP | 1:300 | Mean Mean | −3.6 | N |
| | Conc(μM) | | Inhib (%) | | |
| TBK1 FRET-Z-Lyte SP | Test Conc(μM):ATP | 1:25 | Mean Mean | −0.8 | N |
| | Conc(μM) | | Inhib (%) | | |
| TEK (Tie2) *Mn* FRET-Z-Lyte SP | Test Conc(μM):ATP | 1:10 | Mean Mean | −4.4 | N |
| | Conc(μM) | | Inhib (%) | | |
| TXK FRET-Z-Lyte SP | Test Conc(μM):ATP | 1:100 | Mean Mean | 5.1 | N |
| | Conc(μM) | | Inhib (%) | | |
| TYK2 FRET-Z-Lyte SP | Test Conc(μM):ATP | 1:25 | Mean Mean | 6.0 | N |
| | Conc(μM) | | Inhib (%) | | |
| TYRO3 (RSE) FRET-Z-Lyte SP | Test Conc(μM):ATP | 1:25 | Mean Mean | 9.3 | N |
| | Conc(μM) | | Inhib (%) | | |
| PDGFRB *Mn* FRET-Z-Lyte SP | Test Conc(μM):ATP | 1:100 | Mean Mean | 15.5 | N |
| | Conc(μM) | | Inhib (%) | | |
| PHKG1 FRET-Z-Lyte SP | Test Conc(μM):ATP | 1:75 | Mean Mean | −3.8 | N |
| | Conc(μM) | | Inhib (%) | | |
| PHKG2 FRET-Z-Lyte SP | Test Conc(μM):ATP | 1:10 | Mean Mean | −2.1 | N |
| | Conc(μM) | | Inhib (%) | | |
| PIM1 FRET-Z-Lyte SP | Test Conc(μM):ATP | 1:400 | Mean Mean | 7.8 | N |
| | Conc(μM) | | Inhib (%) | | |
| PIM2 FRET-Z-Lyte SP | Test Conc(μM):ATP | 1:5 | Mean Mean | −0.3 | N |
| | Conc(μM) | | Inhib (%) | | |
| PKN1 (PRK1) FRET-Z-Lyte SP | Test Conc(μM):ATP | 1:50 | Mean Mean | −5.8 | N |
| | Conc(μM) | | Inhib (%) | | |
| PLK1 FRET-Z-Lyte SP | Test Conc(μM):ATP | 1:10 | Mean Mean | 3.0 | N |
| | Conc(μM) | | Inhib (%) | | |
| PLK2 FRET-Z-Lyte SP | Test Conc(μM):ATP | 1:25 | Mean Mean | 1.8 | N |
| | Conc(μM) | | Inhib (%) | | |
| PRKG1 FRET-Z-Lyte SP | Test Conc(μM):ATP | 1:25 | Mean Mean | 5.7 | N |
| | Conc(μM) | | Inhib (%) | | |
| PRKG2 (PKG2) FRET-Z-Lyte SP | Test Conc(μM):ATP | 1:150 | Mean Mean | 2.4 | N |
| | Conc(μM) | | Inhib (%) | | |
| PRKX FRET-Z-Lyte SP | Test Conc(μM):ATP | 1:10 | Mean Mean | −3.2 | N |
| | Conc(μM) | | Inhib (%) | | |
| PTK2 (FAK) FRET-Z-Lyte SP | Test Conc(μM):ATP | 1:50 | Mean Mean | 13.1 | N |
| | Conc(μM) | | Inhib (%) | | |
| RPS6KA5 (MSK1) FRET-Z-Lyte SP | ATP Conc(μM):Test | 1:50 | Mean Mean | 1.6 | N |
| | Conc(μM) | | Inhib (%) | | |
| RPS6KA6 (RSK4) FRET-Z-Lyte SP | Test Conc(μM):ATP | 1:25 | Mean Mean | −9.0 | N |
| | Conc(μM) | | Inhib (%) | | |
| RPS6KB1 (p70S6K) Z-Lyte SP | Test Conc(μM):ATP | 1:10 | Mean Mean | 4.7 | N |
| | Conc(μM) | | Inhib (%) | | |
| SGK (SGK1) FRET-Z-Lyte SP | Test Conc(μM):ATP | 1:25 | Mean Mean | −7.0 | N |
| | Conc(μM) | | Inhib (%) | | |
| SRMS (Srm) FRET-Z-Lyte SP | Test Conc(μM):ATP | 1:150 | Mean Mean | 6.2 | N |
| | Conc(μM) | | Inhib (%) | | |
| SRPK1 FRET-Z-Lyte SP | Test Conc(μM):ATP | 1:25 | Mean Mean | 7.6 | N |
| | Conc(μM) | | Inhib (%) | | |
| SRPK2 FRET-Z-Lyte SP | Test Conc(μM):ATP | 1:25 | Mean Mean | −1.9 | N |
| | Conc(μM) | | Inhib (%) | | |
| STK22B (TSSK2) FRET-Z-Lyte SP | Test Conc(μM):ATP | 1:5 | Mean Mean | 1.2 | N |
| | Conc(μM) | | Inhib (%) | | |
| STK22D (TSSK1) FRET-Z-Lyte SP | Test Conc(μM):ATP | 1:10 | Mean Mean | 18.8 | N |
| | Conc(μM) | | Inhib (%) | | |
| STK23 (MSSK1) FRET-Z-Lyte SP | Test Conc(μM):ATP | 1:75 | Mean Mean | 4.3 | N |
| | Conc(μM) | | Inhib (%) | | |
| STK24 (MST3) FRET-Z-Lyte SP | ATP Conc(μM):Test | 1:50 | Mean Mean | −7.6 | N |
| | Conc(μM) | | Inhib (%) | | |
| SGK2 FRET-Z-Lyte SP | Test Conc(μM):ATP | 1:50 | Mean Mean | 1.2 | N |
| | Conc(μM) | | Inhib (%) | | |
| SGKL (SGK3) FRET-Z-Lyte SP | Test Conc(μM):ATP | 1:10 | Mean Mean | 13.3 | N |
| | Conc(μM) | | Inhib (%) | | |
| SNF1LK2 FRET-Z-Lyte SP | Test Conc(μM):ATP | 1:50 | Mean Mean | 15.9 | N |
| | Conc(μM) | | Inhib (%) | | |
| PTK2B (FAK2) *Mn* Z-Lyte SP | Test Conc(μM):ATP | 1:5 | Mean Mean | 8.5 | N |
| | Conc(μM) | | Inhib (%) | | |
| PTK6 (Brk) *Mn* FRET-Z-Lyte SP | Test Conc(μM):ATP | 1:75 | Mean Mean | 3.5 | N |
| | Conc(μM) | | Inhib (%) | | |
| ROCK1 FRET-Z-Lyte SP | Test Conc(μM):ATP | 1:5 | Mean Mean | 45.9 | N |
| | Conc(μM) | | Inhib (%) | | |
| ROCK2 FRET-Z-Lyte SP | Test Conc(μM):ATP | 1:50 | Mean Mean | −2.1 | N |
| | Conc(μM) | | Inhib (%) | | |
| ROS1 FRET-Z-Lyte SP | Test Conc(μM):ATP | 1:50 | Mean Mean | 10.0 | N |
| | Conc(μM) | | Inhib (%) | | |
| RPS6KA1 (RSK1) FRET-Z-Lyte SP | Test Conc(μM):ATP | 1:5 | Mean Mean | 14.8 | N |
| | Conc(μM) | | Inhib (%) | | |
| RPS6KA2 (RSK3) FRET-Z-Lyte SP | Test Conc(μM):ATP | 1:10 | Mean Mean | 9.0 | N |
| | Conc(μM) | | Inhib (%) | | |
| RPS6KA3 (RSK2) FRET-Z-Lyte SP | Test Conc(μM):ATP | 1:10 | Mean Mean | 0.0 | N |
| | Conc(μM) | | Inhib (%) | | |
| RPS6KA4 (MSK2) FRET-Z-Lyte SP | Test Conc(μM):ATP | 1:10 | Mean Mean | −0.8 | N |
| | Conc(μM) | | Inhib (%) | | |
| ZAP70 *Mn* FRET-Z-Lyte SP | Test Conc(μM):ATP | 1:5 | Mean Mean | 8.1 | N |
| | Conc(μM) | | Inhib (%) | | |
| STK3 (MST2) FRET-Z-Lyte SP | Test Conc(μM):ATP | 1:50 | Mean Mean | 6.9 | N |
| | Conc(μM) | | Inhib (%) | | |
| STK4 (MST1) FRET-Z-Lyte SP | Test Conc(μM):ATP | 1:50 | Mean Mean | −3.1 | N |
| | Conc(μM) | | Inhib (%) | | |
| STK25 (YSK1) FRET-Z-Lyte SP | Test Conc(μM):ATP | 1:75 | Mean Mean | 8.8 | N |
| | Conc(μM) | | Inhib (%) | | |
| YES1 FRET-Z-Lyte SP | Test Conc(μM):ATP | 1:25 | Mean Mean | 5.8 | N |
| | Conc(μM) | | Inhib (%) | | |
| PRKCI (PKC iota) Z-Lyte SP | Test Conc(μM):ATP | 1:25 | Mean Mean | −0.2 | N |
| | Conc(μM) | | Inhib (%) | | |
| PRKCN (PKD3) FRET-Z-Lyte SP | Test Conc(μM):ATP | 1:25 | Mean Mean | 9.0 | N |
| | Conc(μM) | | Inhib (%) | | |
| PRKCQ (PKC theta) Z-Lyte SP | Test Conc(μM):ATP | 1:100 | Mean Mean | 15.6 | N |
| | Conc(μM) | | Inhib (%) | | |
| PRKCD (PKC delta) Z-Lyte SP | Test Conc(μM):ATP | 1:25 | Mean Mean | 15.1 | N |
| | Conc(μM) | | Inhib (%) | | |
| PRKCE (PKC epsilon) Z-Lyte SP | Test Conc(μM):ATP | 1:25 | Mean Mean | 12.2 | N |
| | Conc(μM) | | Inhib (%) | | |
| PRKCG (PKC gamma) Z-Lyte SP | Test Conc(μM):ATP | 1:25 | Mean Mean | 20.4 | N |
| | Conc(μM) | | Inhib (%) | | |
| PAK6 FRET-Z-Lyte SP | Test Conc(μM):ATP | 1:10 | Mean Mean | 15.9 | N |
| | Conc(μM) | | Inhib (%) | | |
| PAK7 (KIAA1264) FRET-Z-Lyte SP | Test Conc(μM):ATP | 1:5 | Mean Mean | 3.9 | N |
| | Conc(μM) | | Inhib (%) | | |
| PASK FRET-Z-Lyte SP | Test Conc(μM):ATP | 1:50 | Mean Mean | 13.7 | N |
| | Conc(μM) | | Inhib (%) | | |
| PRKD1 (PKC mu) Z-Lyte SP | Test Conc(μM):ATP | 1:10 | Mean Mean | 6.5 | N |
| | Conc(μM) | | Inhib (%) | | |
| PRKCZ (PKC zeta) Z-Lyte SP | Test Conc(μM):ATP | 1:5 | Mean Mean | 12.0 | N |
| | Conc(μM) | | Inhib (%) | | |
| PRKD2 (PKD2) Z-Lyte SP | Test Conc(μM):ATP | 1:25 | Mean Mean | 1.5 | N |
| | Conc(μM) | | Inhib (%) | | |
| MKNK1 (MNK1) *Mn* Z-Lyte SP | Test Conc(μM):ATP | 1:100 | Mean Mean | 5.3 | N |
| | Conc(μM) | | Inhib (%) | | |
| MINK1 FRET-Z-Lyte SP | Test Conc(μM):ATP | 1:25 | Mean Mean | 1.6 | N |
| | Conc(μM) | | Inhib (%) | | |
| MST1R (RON) FRET-Z-Lyte SP | Test Conc(μM):ATP | 1:10 | Mean Mean | −8.2 | N |
| | Conc(μM) | | Inhib (%) | | |
| MST4 FRET-Z-Lyte SP | Test Conc(μM):ATP | 1:25 | Mean Mean | 16.7 | N |
| | Conc(μM) | | Inhib (%) | | |
| MUSK *Mn* FRET-Z-Lyte SP | Test Conc(μM):ATP | 1:50 | Mean Mean | 19.5 | N |
| | Conc(μM) | | Inhib (%) | | |
| PLK3 FRET-Z-Lyte SP | Test Conc(μM):ATP | 1:50 | Mean Mean | 5.8 | N |
| | Conc(μM) | | Inhib (%) | | |
| PRKACA (PKA) FRET-Z-Lyte SP | Test Conc(μM):ATP | 1:5 | Mean Mean | 2.2 | N |
| | Conc(μM) | | Inhib (%) | | |
| PRKCA (PKC alpha) Z-Lyte SP | Test Conc(μM):ATP | 1:25 | Mean Mean | 23.3 | N |
| | Conc(μM) | | Inhib (%) | | |
| PRKCB1 (PKC beta I) Z-Lyte SP | Test Conc(μM):ATP | 1:200 | Mean Mean | 27.7 | N |
| | Conc(μM) | | Inhib (%) | | |
| PRKCH (PKC eta) Z-Lyte SP | Test Conc(μM):ATP | 1:25 | Mean Mean | 9.3 | N |
| | Conc(μM) | | Inhib (%) | | |
| EGFR (ErbB1) *Mn* Z-Lyte SP | Test Conc(μM):ATP | 1:10 | Mean Mean | 1.5 | N |
| | Conc(μM) | | Inhib (%) | | |
| ABL1 Hu Phos FRET-Z-Lyte SP | Test Conc(μM):ATP | 1:10 | Mean Mean | 2.8 | N |
| | Conc(μM) | | Inhib (%) | | |
| AMPK A2/B1/G1 FRET-Z-Lyte SP | Test Conc(μM):ATP | 1:150 | Mean Mean | −3.5 | N |
| | Conc(μM) | | Inhib (%) | | |
| bRAF Cascade FRET-Z-Lyte SP | Test Conc(μM):ATP | 1:100 | Mean Mean | 10.3 | N |
| | Conc(μM) | | Inhib (%) | | |
| DDR2 Hu Bind FRET-Lantha SP | Test Conc(μM) | 1 | Mean Mean | 65.5 | N |
| | | | Inhib (%) | | |
| FES (FPS) FRET-Z-Lyte SP | Test Conc(μM):ATP | 1:25 | Mean Mean | 10.0 | N |
| | Conc(μM) | | Inhib (%) | | |
| FGFR3 *Mn* FRET-Z-Lyte SP | Test Conc(μM):ATP | 1:75 | Mean Mean | −2.6 | N |
| | Conc(μM) | | Inhib (%) | | |
| FLT1 (VEGFR1) *Mn* Z-Lyte SP | Test Conc(μM):ATP | 1:150 | Mean Mean | 11.5 | N |
| | Conc(μM) | | Inhib (%) | | |
| FLT3 Hu Phos FRET-Z-Lyte SP | Test Conc(μM):ATP | 1:500 | Mean Mean | −9.3 | N |
| | Conc(μM) | | Inhib (%) | | |
| KIT *Mn* FRET-Z-Lyte SP | Test Conc(μM):ATP | 1:300 | Mean Mean | 0.0 | N |
| | Conc(μM) | | Inhib (%) | | |
| LYN A Hu Phos FRET-Z-Lyte SP | Test Conc(μM):ATP | 1:25 | Mean Mean | 6.3 | N |
| | Conc(μM) | | Inhib (%) | | |
| MET (cMet) FRET-Z-Lyte SP | Test Conc(μM):ATP | 1:50 | Mean Mean | 0.4 | N |
| | Conc(μM) | | Inhib (%) | | |
| PDK1 Cascade FRET-Z-Lyte SP | Test Conc(μM):ATP | 1:100 | Mean Mean | 3.7 | N |
| | Conc(μM) | | Inhib (%) | | |
| RET Hu Phos FRET-Z-Lyte SP | Test Conc(μM):ATP | 1:10 | Mean Mean | 22.8 | N |
| | Conc(μM) | | Inhib (%) | | |
| SRC Hu Phos FRET-Z-Lyte SP | Test Conc(μM):ATP | 1:50 | Mean Mean | 14.3 | N |
| | Conc(μM) | | Inhib (%) | | |
| TGFBR1 (ALK5) FRET-Lantha SP | Test Conc(μM) | 1 | Mean Mean | 5.3 | N |
| | | | Inhib (%) | | |
| MAPK14 (p38a)Cascade Z-Lyte SP | Test Conc(μM):ATP | 1:100 | Mean Mean | 59.0 | N |
| | Conc(μM) | | Inhib (%) | | |
| MAPK14 (p38a) Direct Z-Lyte SP | Test Conc(μM):ATP | 1:500 | Mean Mean | 79.8 | A |
| | Conc(μM) | | Inhib (%) | | |
| PDK1 (Direct) FRET-Z-lyte SP | Test Conc(μM):ATP | 1:25 | Mean Mean | 0.4 | N |
| | Conc(μM) | | Inhib (%) | | |
| ACVRL1 (ALK1) FRET-Lantha SP | Test Conc(μM) | 1 | Mean Mean | 7.2 | N |
| | | | Inhib (%) | | |
| CLK1 FRET-Z-Lyte SP | Test Conc(μM):ATP | 1:25 | Mean Mean | 5.3 | N |
| | Conc(μM) | | Inhib (%) | | |
| CLK4 FRET-Lantha SP | Test Conc(μM) | 1 | Mean Mean | 6.4 | N |
| | | | Inhib (%) | | |
| MAP4K3 (GLK) FRET-Lantha SP | Test Conc(μM) | 1 | Mean Mean | 7.7 | N |
| | | | Inhib (%) | | |
| ACVR1 (ALK2) FRET-Lantha SP | Test Conc(μM) | 1 | Mean Mean | 2.3 | N |
| | | | Inhib (%) | | |
| AMPK A1/B1/G1 FRET-Z-lyte SP | Test Conc(μM):ATP | 1:50 | Mean Mean | 0.3 | N |
| | Conc(μM) | | Inhib (%) | | |
| AMPK (A1/B1/G2) FRET-Lantha SP | Test Conc(μM) | 1 | Mean Mean | 5.6 | N |
| | | | Inhib (%) | | |
| AMPK (A1/B1/G3) FRET-Lantha SP | Test Conc(μM) | 1 | Mean Mean | −10.3 | N |
| | | | Inhib (%) | | |
| AMPK (A1/B2/G1) FRET-Lantha SP | Test Conc(μM) | 1 | Mean Mean | 0.0 | N |
| | | | Inhib (%) | | |
| AMPK (A2/B2/G1) FRET-Lantha SP | Test Conc(μM) | 1 | Mean Mean | −0.8 | N |
| | | | Inhib (%) | | |
| AMPK (A2/B2/G2) FRET-Lantha SP | Test Conc(μM) | 1 | Mean Mean | −1.1 | N |
| | | | Inhib (%) | | |
| bRAF FRET-Lantha SP | Test Conc(μM) | 1 | Mean Mean | 66.8 | N |
| | | | Inhib (%) | | |
| CDK1/cyclin A2 FRET-Lantha SP | Test Conc(μM) | 1 | Mean Mean | −1.0 | N |
| | | | Inhib (%) | | |
| CDK11(Inactive) FRET-Lantha SP | Test Conc(μM) | 1 | Mean Mean | −0.2 | N |
| | | | Inhib (%) | | |
| CDK14(PFTK1)/cyclinY Lantha SP | Test Conc(μM) | 1 | Mean Mean | 3.5 | N |
| | | | Inhib (%) | | |
| CDK2/cyclin A1 FRET-Lantha SP | Test Conc(μM) | 1 | Mean Mean | −1.8 | N |
| | | | Inhib (%) | | |
| CDK3/cyclin E1 FRET-Lantha SP | Test Conc(μM) | 1 | Mean Mean | 6.0 | N |
| | | | Inhib (%) | | |
| CDK5/p25 FRET-Z-Lyte SP | Test Conc(μM):ATP | 1:10 | Mean Mean | 2.0 | N |
| | Conc(μM) | | Inhib (%) | | |
| CDK5/p35 FRET-Z-Lyte SP | Test Conc(μM):ATP | 1:10 | Mean Mean | 0.9 | N |
| | Conc(μM) | | Inhib (%) | | |
| CDK9/cyclin K FRET-Lantha SP | Test Conc(μM) | 1 | Mean Mean | −1.9 | N |
| | | | Inhib (%) | | |
| FYN A FRET-Lantha SP | Test Conc(μM) | 1 | Mean Mean | 4.8 | N |
| | | | Inhib (%) | | |
| JAK2 JH1 JH2 FRET-Z-Lyte SP | ATP Conc(μM):Test | 1:25 | Mean Mean | −0.1 | N |
| | Conc(μM) | | Inhib (%) | | |
| LRRK2 FL FRET-Adapta SP | Test Conc(μM):ATP | 1:50 | Mean Mean | 0.5 | N |
| | Conc(μM) | | Inhib (%) | | |
| LYN B FRET-Z-Lyte SP | Test Conc(μM):ATP | 1:25 | Mean Mean | 16.3 | N |
| | Conc(μM) | | Inhib (%) | | |
| MYLK2 FRET-Z-lyte SP | Test Conc(μM):ATP | 1:300 | Mean Mean | 7.4 | N |
| | Conc(μM) | | Inhib (%) | | |
| PDGFRA *Mn* FRET-Z-Lyte SP | Test Conc(μM):ATP | 1:10 | Mean Mean | 2.4 | N |
| | Conc(μM) | | Inhib (%) | | |
| PRKCB2 (PKC Beta II) Z-Lyte SP | Test Conc(μM):ATP | 1:200 | Mean Mean | −11.1 | N |
| | Conc(μM) | | Inhib (%) | | |
| CDK2/Cyclin E1 FRET-Lantha SP | Test Conc(μM) | 1 | Mean Mean | 13.8 | N |
| | | | Inhib (%) | | |
| CDK2/Cyclin O FRET-Lantha SP | Test Conc(μM) | 1 | Mean Mean | 2.7 | N |
| | | | Inhib (%) | | |
| SIK1 FRET-Lantha SP | Test Conc(μM) | 1 | Mean Mean | −0.5 | N |
| | | | Inhib (%) | | |
| STK38 FRET-Lantha SP | Test Conc(μM) | 1 | Mean Mean | −9.2 | N |
| | | | Inhib (%) | | |
| MYLK3 Hu Bind FRET-Lantha SP | Test Conc(μM) | 1 | Mean Mean | 4.5 | N |
| | | | Inhib (%) | | |
| SLK Hu Bind FRET-Lantha SP | Test Conc(μM) | 1 | Mean Mean | −3.6 | N |
| | | | Inhib (%) | | |
| AMPK A1/B2/G3 FRET-Z-Lyte SP | Test Conc(μM):ATP | 1:10 | Mean Mean | 20.8 | N |
| | Conc(μM) | | Inhib (%) | | |
| AMPK A2/B1/G2 FRET-Z-Lyte SP | Test Conc(μM):ATP | 1:10 | Mean Mean | 11.4 | N |
| | Conc(μM) | | Inhib (%) | | |
| AMPK A2/B1/G3 FRET-Z-Lyte SP | Test Conc(μM):ATP | 1:25 | Mean Mean | 7.8 | N |
| | Conc(μM) | | Inhib (%) | | |
| AMPK A2/B2/G3 FRET-Z-Lyte SP | Test Conc(μM):ATP | 1:50 | Mean Mean | 3.8 | N |
| | Conc(μM) | | Inhib (%) | | |
| MAPK7 (ERK5) FRET-Z-Lyte SP | Test Conc(μM):ATP | 1:10 | Mean Mean | 0.2 | N |
| | Conc(μM) | | Inhib (%) | | |
| CAMK1G FRET-Z-Lyte SP | Test Conc(μM):ATP | 1:300 | Mean Mean | 5.8 | N |
| | Conc(μM) | | Inhib (%) | | |
| CDC42 BPG FRET-Z-Lyte SP | Test Conc(μM):ATP | 1:5 | Mean Mean | 2.2 | N |
| | Conc(μM) | | Inhib (%) | | |
| CDKL5 FRET-Z-Lyte SP | Test Conc(μM):ATP | 1:500 | Mean Mean | 13.9 | N |
| | Conc(μM) | | Inhib (%) | | |
| CSNK1A1L FRET-Z-Lyte SP | Test Conc(μM):ATP | 1:5 | Mean Mean | 2.0 | N |
| | Conc(μM) | | Inhib (%) | | |
| DCAMKL1 (DCLK1) FRET-Z-Lyte SP | Test Conc(μM):ATP | 1:300 | Mean Mean | 31.0 | N |
| | Conc(μM) | | Inhib (%) | | |
| CDK17/cyclin Y FRET-Z-Lyte SP | Test Conc(μM):ATP | 1:50 | Mean Mean | 0.9 | N |
| | Conc(μM) | | Inhib (%) | | |
| CDK18/cyclin Y FRET-Z-Lyte SP | Test Conc(μM):ATP | 1:75 | Mean Mean | 2.7 | N |
| | Conc(μM) | | Inhib (%) | | |
| MAP3K19 (YSK4) FRET-Z-Lyte SP | Test Conc(μM):ATP | 1:5 | Mean Mean | −1.6 | N |
| | Conc(μM) | | Inhib (%) | | |
| NIM1K FRET-Z-Lyte SP | Test Conc(μM):ATP | 1:10 | Mean Mean | −0.9 | N |
| | Conc(μM) | | Inhib (%) | | |
| PIM3 FRET-Z-Lyte SP | Test Conc(μM):ATP | 1:1000 | Mean Mean | −1.4 | N |
| | Conc(μM) | | Inhib (%) | | |
| SBK1 FRET-Z-Lyte SP | Test Conc(μM):ATP | 1:5 | Mean Mean | 3.3 | N |
| | Conc(μM) | | Inhib (%) | | |
| TNK1 FRET-Z-Lyte SP | Test Conc(μM):ATP | 1:200 | Mean Mean | −8.8 | N |
| | Conc(μM) | | Inhib (%) | | |
| PI4K2A *Mn* FRET-Adapta SP | Test Conc(μM):ATP | 1:10 | Mean Mean | −2.4 | N |
| | Conc(μM) | | Inhib (%) | | |
| PI4K2B *Mn* FRET-Adapta SP | Test Conc(μM):ATP | 1:10 | Mean Mean | −15.8 | N |
| | Conc(μM) | | Inhib (%) | | |
| PIK3C2G FRET-Adapta SP | Test Conc(μM):ATP | 1:10 | Mean Mean | 5.6 | N |
| | Conc(μM) | | Inhib (%) | | |
| PIK3CA/PIK3R3 FRET-Adapta SP | Test Conc(μM):ATP | 1:10 | Mean Mean | −3.0 | N |
| | Conc(μM) | | Inhib (%) | | |
| PIK3CB/PIK3R2 FRET-Adapta SP | Test Conc(μM):ATP | 1:10 | Mean Mean | 8.0 | N |
| | Conc(μM) | | Inhib (%) | | |
| AAK1 FRET-Lantha SP | Test Conc(μM) | 1 | Mean Mean | 5.8 | N |
| | | | Inhib (%) | | |
| ADCK3 FRET-Lantha SP | Test Conc(μM) | 1 | Mean Mean | −1.6 | N |
| | | | Inhib (%) | | |
| ERN1 FRET-Lantha SP | Test Conc(μM) | 1 | Mean Mean | −3.4 | N |
| | | | Inhib (%) | | |
| GAK FRET-Lantha SP | Test Conc(μM) | 1 | Mean Mean | 29.0 | N |
| | | | Inhib (%) | | |
| HUNK FRET-Lantha SP | Test Conc(μM) | 1 | Mean Mean | 5.7 | N |
| | | | Inhib (%) | | |
| IRAK3 FRET-Lantha SP | Test Conc(μM) | 1 | Mean Mean | 14.4 | N |
| | | | Inhib (%) | | |
| MAP2K4 (MEK4) FRET-Lantha SP | Test Conc(μM) | 1 | Mean Mean | −9.5 | N |
| | | | Inhib (%) | | |
| MAP2K5 (MEK5) FRET-Lantha SP | Test Conc(μM) | 1 | Mean Mean | −1.6 | N |
| | | | Inhib (%) | | |
| MLK4 FRET-Lantha SP | Test Conc(μM) | 1 | Mean Mean | −1.5 | N |
| | | | Inhib (%) | | |
| MYLK4 FRET-Lantha SP | Test Conc(μM) | 1 | Mean Mean | 5.1 | N |
| | | | Inhib (%) | | |
| MYO3A (MYO3 alpha) Lantha SP | Test Conc(μM) | 1 | Mean Mean | 10.8 | N |
| | | | Inhib (%) | | |
| PKMYT1 FRET-Lantha SP | Test Conc(μM) | 1 | Mean Mean | 9.3 | N |
| | | | Inhib (%) | | |
| TESK1 FRET-Lantha SP | Test Conc(μM) | 1 | Mean Mean | −2.5 | N |
| | | | Inhib (%) | | |
| VRK2 FRET-Lantha SP | Test Conc(μM) | 1 | Mean Mean | −5.7 | N |
| | | | Inhib (%) | | |
| KSR2 FRET-Z-Lyte SP | Test Conc(μM):ATP | 1:10 | Mean Mean | 3.9 | N |
| | Conc(μM) | | Inhib (%) | | |
| PEAK1 FRET-Z-Lyte SP | Test Conc(μM):ATP | 1:50 | Mean Mean | 0.8 | N |
| | Conc(μM) | | Inhib (%) | | |
| RPS6KB2 FRET-Z-Lyte SP | Test Conc(μM):ATP | 1:50 | Mean Mean | 7.8 | N |
| | Conc(μM) | | Inhib (%) | | |
| ERN2 FRET-Lantha SP | Test Conc(μM) | 1 | Mean Mean | −3.5 | N |
| | | | Inhib (%) | | |
| MASTL FRET-Lantha SP | Test Conc(μM) | 1 | Mean Mean | 2.3 | N |
| | | | Inhib (%) | | |
| NEK8 FRET-Lantha SP | Test Conc(μM) | 1 | Mean Mean | −9.5 | N |
| | | | Inhib (%) | | |
| AMPK A1/B2/G2 FRET-Z-Lyte SP | Test Conc(μM):ATP | 1:10 | Mean Mean | 12.2 | N |
| | Conc(μM) | | Inhib (%) | | |
| CDK6 Cyclin D1 FRET-Adapta SP | Test Conc(μM):ATP | 1:10 | Mean Mean | −1.8 | N |
| | Conc(μM) | | Inhib (%) | | |
| PIP4K2A FRET-Adapta SP | Test Conc(μM):ATP | 1:10 | Mean Mean | 2.2 | N |
| | Conc(μM) | | Inhib (%) | | |
| PIP5K1A FRET-Adapta SP | Test Conc(μM):ATP | 1:10 | Mean Mean | −2.9 | N |
| | Conc(μM) | | Inhib (%) | | |
| PIP5K1B FRET-Adapta SP | Test Conc(μM):ATP | 1:10 | Mean Mean | 4.5 | N |
| | Conc(μM) | | Inhib (%) | | |
| PIP5K1C FRET-Adapta SP | Test Conc(μM):ATP | 1:10 | Mean Mean | −4.5 | N |
| | Conc(μM) | | Inhib (%) | | |
| WNK1 FRET-Lantha SP | Test Conc(μM) | 1 | Mean Mean | 2.7 | N |
| | | | Inhib (%) | | |
| CDK4 Cyclin D1 *Mn* Adapta SP | Test Conc(μM):ATP | 1:10 | Mean Mean | 7.8 | N |
| | Conc(μM) | | Inhib (%) | | |
| CDK4 Cyclin D3 *Mn* Adapta SP | Test Conc(μM):ATP | 1:10 | Mean Mean | 5.0 | N |
| | Conc(μM) | | Inhib (%) | | |
| CDK13 Cyclin K FRET-Lantha SP | Test Conc(μM) | 1 | Mean Mean | 5.4 | N |
| | | | Inhib (%) | | |
| CDK11 Cyclin C FRET-Lantha SP | Test Conc(μM) | 1 | Mean Mean | 2.1 | N |
| | | | Inhib (%) | | |

TABLE 3

| | | | Princ. | Princ. | Dist |
|---|---|---|---|---|---|
| | | Split | Result | Result | Act |
| Test Short Name | Split | Value | Type | Value | Flag |
| LRRK2 Hu Phos FRET-Adapta SP | Test Conc(μM):ATP | 1:75 | Mean Mean | 6.1 | N |
| | Conc(μM) | | Inhib (%) | | |
| PI4KB FRET-Adapta SP | Test Conc(μM):ATP | 1:5 | Mean Mean | 0.5 | N |
| | Conc(μM) | | Inhib (%) | | |
| PIK3C2A FRET-Adapta SP | Test Conc(μM):ATP | 1:25 | Mean Mean | 8.7 | N |
| | Conc(μM) | | Inhib (%) | | |
| PIK3C2B FRET-Adapta SP | Test Conc(μM):ATP | 1:10 | Mean Mean | −0.9 | N |
| | Conc(μM) | | Inhib (%) | | |
| PIK3C3 (hVPS34) *Mn* Adapta SP | Test Conc(μM):ATP | 1:10 | Mean Mean | 1.1 | N |
| | Conc(μM) | | Inhib (%) | | |
| PIK3CA/PIK3R1 FRET-Adapta SP | Test Conc(μM):ATP | 1:25 | Mean Mean | 0.0 | N |
| | Conc(μM) | | Inhib (%) | | |
| CAMK1 (CaMK1) FRET-Adapta SP | Test Conc(μM):ATP | 1:10 | Mean Mean | 7.3 | N |
| | Conc(μM) | | Inhib (%) | | |
| CDK7/cyclin H/MNAT1 Adapta SP | Test Conc(μM):ATP | 1:150 | Mean Mean | −2.2 | N |
| | Conc(μM) | | Inhib (%) | | |
| CDK9/cyclin T1 FRET-Adapta SP | Test Conc(μM):ATP | 1:25 | Mean Mean | 6.2 | N |
| | Conc(μM) | | Inhib (%) | | |
| CHUK(IKK alpha) FRET-Adapta SP | Test Conc(μM):ATP | 1:10 | Mean Mean | −6.2 | N |
| | Conc(μM) | | Inhib (%) | | |
| DAPK1 FRET-Adapta SP | Test Conc(μM):ATP | 1:5 | Mean Mean | −2.6 | N |
| | Conc(μM) | | Inhib (%) | | |
| GSG2 (Haspin) FRET-Adapta SP | Test Conc(μM):ATP | 1:25 | Mean Mean | −1.9 | N |
| | Conc(μM) | | Inhib (%) | | |
| IRAK1 FRET-Adapta SP | Test Conc(μM):ATP | 1:25 | Mean Mean | 5.1 | N |
| | Conc(μM) | | Inhib (%) | | |
| NUAK1 (ARK5) FRET-Adapta SP | Test Conc(μM):ATP | 1:25 | Mean Mean | −3.5 | N |
| | Conc(μM) | | Inhib (%) | | |
| PI4KA FRET-Adapta SP | Test Conc(μM):ATP | 1:10 | Mean Mean | −6.0 | N |
| | Conc(μM) | | Inhib (%) | | |
| PIK3CB FRET-Adapta SP | Test Conc(μM):ATP | 1:150 | Mean Mean | −2.3 | N |
| | Conc(μM) | | Inhib (%) | | |
| PIK3CD/PIK3R1 FRET-Adapta SP | Test Conc(μM):ATP | 1:75 | Mean Mean | 23.8 | N |
| | Conc(μM) | | Inhib (%) | | |
| SPHK1 FRET-Adapta SP | Test Conc(μM):ATP | 1:10 | Mean Mean | −0.7 | N |
| | Conc(μM) | | Inhib (%) | | |
| SPHK2 FRET-Adapta SP | Test Conc(μM):ATP | 1:10 | Mean Mean | −1.2 | N |
| | Conc(μM) | | Inhib (%) | | |
| PIK3CG (p110 gamma) Adapta SP | Test Conc(μM):ATP | 1:25 | Mean Mean | 9.1 | N |
| | Conc(μM) | | Inhib (%) | | |
| DDR1 FRET-Lantha SP | Test Conc(μM) | 1 | Mean Mean | 1.7 | N |
| | | | Inhib (%) | | |
| EIF2AK2 (PKR) FRET-Lantha SP | Test Conc(μM) | 1 | Mean Mean | 3.6 | N |
| | | | Inhib (%) | | |
| EPHA3 FRET-Lantha SP | Test Conc(μM) | 1 | Mean Mean | 27.9 | N |
| | | | Inhib (%) | | |
| EPHA6 FRET-Lantha SP | Test Conc(μM) | 1 | Mean Mean | 54.2 | N |
| | | | Inhib (%) | | |
| DMPK FRET-Lantha SP | Test Conc(μM) | 1 | Mean Mean | 0.5 | N |
| | | | Inhib (%) | | |
| DYRK2 FRET-Lantha SP | Test Conc(μM) | 1 | Mean Mean | 9.6 | N |
| | | | Inhib (%) | | |
| CDK5 (Inactive) FRET-Lantha SP | Test Conc(μM) | 1 | Mean Mean | 17.2 | N |
| | | | Inhib (%) | | |
| CDK8/cyclin C FRET-Lantha SP | Test Conc(μM) | 1 | Mean Mean | 5.9 | N |
| | | | Inhib (%) | | |
| CDK9 (Inactive) FRET-Lantha SP | Test Conc(μM) | 1 | Mean Mean | 6.9 | N |
| | | | Inhib (%) | | |
| CDC7/DBF4 FRET-Lantha SP | Test Conc(μM) | 1 | Mean Mean | 0.9 | N |
| | | | Inhib (%) | | |
| CDK16(PCTK1)/cyclinY Lantha SP | Test Conc(μM) | 1 | Mean Mean | 0.2 | N |
| | | | Inhib (%) | | |
| CAMK2G FRET-Lantha SP | Test Conc(μM) | 1 | Mean Mean | 1.3 | N |
| | | | Inhib (%) | | |
| CAMKK1 (Alpha) FRET-Lantha SP | Test Conc(μM) | 1 | Mean Mean | −6.4 | N |
| | | | Inhib (%) | | |
| ACVR2A FRET-Lantha SP | Test Conc(μM) | 1 | Mean Mean | 71.5 | N |
| | | | Inhib (%) | | |
| ACVR2B FRET-Lantha SP | Test Conc(μM) | 1 | Mean Mean | 95.6 | A |
| | | | Inhib (%) | | |
| BMPR1A (ALK3) FRET-Lantha SP | Test Conc(μM) | 1 | Mean Mean | 83.6 | A |
| | | | Inhib (%) | | |
| BMPR1B (ALK6) FRET-Lantha SP | Test Conc(μM) | 1 | Mean Mean | 107.0 | A |
| | | | Inhib (%) | | |
| CAMKK2 (Beta) FRET-Lantha SP | Test Conc(μM) | 1 | Mean Mean | −3.4 | N |
| | | | Inhib (%) | | |
| CASK FRET-Lantha SP | Test Conc(μM) | 1 | Mean Mean | 12.1 | N |
| | | | Inhib (%) | | |
| DAPK2 FRET-Lantha SP | Test Conc(μM) | 1 | Mean Mean | −0.8 | N |
| | | | Inhib (%) | | |
| BMPR2 FRET-Lantha SP | Test Conc(μM) | 1 | Mean Mean | 2.7 | N |
| | | | Inhib (%) | | |
| BRSK2 FRET-Lantha SP | Test Conc(μM) | 1 | Mean Mean | 10.2 | N |
| | | | Inhib (%) | | |
| MAP2K6 (MKK6) FRET-Lantha SP | Test Conc(μM) | 1 | Mean Mean | 2.3 | N |
| | | | Inhib (%) | | |
| LIMK2 FRET-Lantha SP | Test Conc(μM) | 1 | Mean Mean | 22.2 | N |
| | | | Inhib (%) | | |
| MAP2K1 (MEK1) FRET-Lantha SP | Test Conc(μM) | 1 | Mean Mean | −0.5 | N |
| | | | Inhib (%) | | |
| GRK1 FRET-Lantha SP | Test Conc(μM) | 1 | Mean Mean | 3.7 | N |
| | | | Inhib (%) | | |
| ICK FRET-Lantha SP | Test Conc(μM) | 1 | Mean Mean | 6.0 | N |
| | | | Inhib (%) | | |
| EPHA7 FRET-Lantha SP | Test Conc(μM) | 1 | Mean Mean | 10.4 | N |
| | | | Inhib (%) | | |
| LATS2 FRET-Lantha SP | Test Conc(μM) | 1 | Mean Mean | 4.9 | N |
| | | | Inhib (%) | | |
| LIMK1 FRET-Lantha SP | Test Conc(μM) | 1 | Mean Mean | 1.8 | N |
| | | | Inhib (%) | | |
| MAP2K2 (MEK2) FRET-Lantha SP | Test Conc(μM) | 1 | Mean Mean | −0.2 | N |
| | | | Inhib (%) | | |
| LATS1 FRET-Lantha SP | Test Conc(μM) | 1 | Mean Mean | 17.2 | N |
| | | | Inhib (%) | | |
| MAPK10 (JNK3) FRET-Lantha SP | Test Conc(μM) | 1 | Mean Mean | 5.5 | N |
| | | | Inhib (%) | | |
| MAPK15 (ERK7) FRET-Lantha SP | Test Conc(μM) | 1 | Mean Mean | 1.5 | N |
| | | | Inhib (%) | | |
| MAP3K2 (MEKK2) FRET-Lantha | Test Conc(μM) | 1 | Mean Mean | 5.7 | N |
| SP | | | Inhib (%) | | |
| MAP3K3 (MEKK3) FRET-Lantha | Test Conc(μM) | 1 | Mean Mean | 14.8 | N |
| SP | | | Inhib (%) | | |
| MAP3K5 (ASK1) FRET-Lantha SP | Test Conc(μM) | 1 | Mean Mean | 6.1 | N |
| | | | Inhib (%) | | |
| MAP3K11 (MLK3) FRET-Lantha SP | Test Conc(μM) | 1 | Mean Mean | −0.8 | N |
| | | | Inhib (%) | | |
| MAP3K14 (NIK) FRET-Lantha SP | Test Conc(μM) | 1 | Mean Mean | 4.9 | N |
| | | | Inhib (%) | | |
| MAP3K10 (MLK2) FRET-Lantha SP | Test Conc(μM) | 1 | Mean Mean | 2.7 | N |
| | | | Inhib (%) | | |
| MAP4K1 (HPK1) FRET-Lantha SP | Test Conc(μM) | 1 | Mean Mean | 11.3 | N |
| | | | Inhib (%) | | |
| MAPK8 (JNK1) FRET-Lantha SP | Test Conc(μM) | 1 | Mean Mean | 7.0 | N |
| | | | Inhib (%) | | |
| MAPK9 (JNK2) FRET-Lantha SP | Test Conc(μM) | 1 | Mean Mean | 5.2 | N |
| | | | Inhib (%) | | |
| STK16 (PKL12) FRET-Lantha SP | Test Conc(μM) | 1 | Mean Mean | 3.4 | N |
| | | | Inhib (%) | | |
| PRKACG FRET-Lantha SP | Test Conc(μM) | 1 | Mean Mean | 2.2 | N |
| | | | Inhib (%) | | |
| MYLK (MLCK) FRET-Lantha SP | Test Conc(μM) | 1 | Mean Mean | −0.5 | N |
| | | | Inhib (%) | | |
| MYO3B FRET-Lantha SP | Test Conc(μM) | 1 | Mean Mean | 4.1 | N |
| | | | Inhib (%) | | |
| NLK FRET-Lantha SP | Test Conc(μM) | 1 | Mean Mean | 88.8 | A |
| | | | Inhib (%) | | |
| NUAK2 FRET-Lantha SP | Test Conc(μM) | 1 | Mean Mean | 9.4 | N |
| | | | Inhib (%) | | |
| MKNK2 (MNK2) FRET-Lantha SP | Test Conc(μM) | 1 | Mean Mean | 2.7 | N |
| | | | Inhib (%) | | |
| MAP3K7/MAP3K7IP1 Lantha SP | Test Conc(μM) | 1 | Mean Mean | 8.1 | N |
| | | | Inhib (%) | | |
| PLK4 FRET-Lantha SP | Test Conc(μM) | 1 | Mean Mean | 6.1 | N |
| | | | Inhib (%) | | |
| PRKACB FRET-Lantha SP | Test Conc(μM) | 1 | Mean Mean | 10.9 | N |
| | | | Inhib (%) | | |
| RIPK2 FRET-Lantha SP | Test Conc(μM) | 1 | Mean Mean | 64.0 | N |
| | | | Inhib (%) | | |
| RIPK3 FRET-Lantha SP | Test Conc(μM) | 1 | Mean Mean | 76.6 | A |
| | | | Inhib (%) | | |
| SIK3 FRET-Lantha SP | Test Conc(μM) | 1 | Mean Mean | −4.7 | N |
| | | | Inhib (%) | | |
| PKN2 (PRK2) FRET-lantha SP | Test Conc(μM) | 1 | Mean Mean | 5.1 | N |
| | | | Inhib (%) | | |
| STK17A (DRAK1) FRET-Lantha SP | Test Conc(μM) | 1 | Mean Mean | −1.2 | N |
| | | | Inhib (%) | | |
| TNIK FRET-Lantha SP | Test Conc(μM) | 1 | Mean Mean | 58.4 | N |
| | | | Inhib (%) | | |
| STK32C (YANK3) FRET-Lantha SP | Test Conc(μM) | 1 | Mean Mean | 7.8 | N |
| | | | Inhib (%) | | |
| STK33 FRET-Lantha SP | Test Conc(μM) | 1 | Mean Mean | −6.6 | N |
| | | | Inhib (%) | | |
| STK38L (NDR2) FRET-Lantha SP | Test Conc(μM) | 1 | Mean Mean | 5.5 | N |
| | | | Inhib (%) | | |
| STK39 (STLK3) FRET-Lantha SP | Test Conc(μM) | 1 | Mean Mean | 5.1 | N |
| | | | Inhib (%) | | |
| STK32B (YANK2) FRET-Lantha SP | Test Conc(μM) | 1 | Mean Mean | 2.1 | N |
| | | | Inhib (%) | | |
| STK17B (DRAK2) FRET-Lantha SP | Test Conc(μM) | 1 | Mean Mean | 2.0 | N |
| | | | Inhib (%) | | |
| TAOK3 (JIK) FRET-Lantha SP | Test Conc(μM) | 1 | Mean Mean | 1.7 | N |
| | | | Inhib (%) | | |
| TEC FRET-Lantha SP | Test Conc(μM) | 1 | Mean Mean | −1.4 | N |
| | | | Inhib (%) | | |
| TGFBR2 FRET-Lantha SP | Test Conc(μM) | 1 | Mean Mean | 67.0 | N |
| | | | Inhib (%) | | |
| TLK1 FRET-Lantha SP | Test Conc(μM) | 1 | Mean Mean | −4.5 | N |
| | | | Inhib (%) | | |
| TLK2 FRET-Lantha SP | Test Conc(μM) | 1 | Mean Mean | −4.8 | N |
| | | | Inhib (%) | | |
| TNK2 (ACK) FRET-Lantha SP | Test Conc(μM) | 1 | Mean Mean | 6.5 | N |
| | | | Inhib (%) | | |
| TTK FRET-Lantha SP | Test Conc(μM) | 1 | Mean Mean | −2.0 | N |
| | | | Inhib (%) | | |
| ULK3 FRET-Lantha SP | Test Conc(μM) | 1 | Mean Mean | −2.4 | N |
| | | | Inhib (%) | | |
| WEE1 FRET-Lantha SP | Test Conc(μM) | 1 | Mean Mean | 1.2 | N |
| | | | Inhib (%) | | |
| ULK1 FRET-Lantha SP | Test Conc(μM) | 1 | Mean Mean | 0.1 | N |
| | | | Inhib (%) | | |
| ULK2 FRET-Lantha SP | Test Conc(μM) | 1 | Mean Mean | −1.0 | N |
| | | | Inhib (%) | | |
| TAOK1 FRET-Lantha SP | Test Conc(μM) | 1 | Mean Mean | 33.0 | N |
| | | | Inhib (%) | | |
| WNK2 FRET-Lantha SP | Test Conc(μM) | 1 | Mean Mean | 5.8 | N |
| | | | Inhib (%) | | |
| WNK3 FRET-Lantha SP | Test Conc(μM) | 1 | Mean Mean | −2.5 | N |
| | | | Inhib (%) | | |
| ZAK FRET-Lantha SP | Test Conc(μM) | 1 | Mean Mean | 78.3 | A |
| | | | Inhib (%) | | |
| ABL2 (Arg) FRET-Z-Lyte SP | Test Conc(μM):ATP | 1:25 | Mean Mean | 82.6 | A |
| | Conc(μM) | | Inhib (%) | | |
| ACVR1B (ALK4) *Mn* Z-Lyte SP | Test Conc(μM):ATP | 1:5 | Mean Mean | 97.8 | A |
| | Conc(μM) | | Inhib (%) | | |
| EPHB4 FRET-Z-Lyte SP | Test Conc(μM):ATP | 1:100 | Mean Mean | 58.1 | N |
| | Conc(μM) | | Inhib (%) | | |
| GRK6 FRET-Z-Lyte SP | Test Conc(μM):ATP | 1:10 | Mean Mean | 2.1 | N |
| | Conc(μM) | | Inhib (%) | | |
| EPHB3 FRET-Z-Lyte SP | Test Conc(μM):ATP | 1:75 | Mean Mean | 1.6 | N |
| | Conc(μM) | | Inhib (%) | | |
| GRK7 FRET-Z-Lyte SP | Test Conc(μM):ATP | 1:10 | Mean Mean | 8.3 | N |
| | Conc(μM) | | Inhib (%) | | |
| GSK3A FRET-Z-Lyte SP | Test Conc(μM):ATP | 1:10 | Mean Mean | 3.2 | N |
| | Conc(μM) | | Inhib (%) | | |
| GSK3B FRET-Z-Lyte SP | Test Conc(μM):ATP | 1:10 | Mean Mean | 4.9 | N |
| | Conc(μM) | | Inhib (%) | | |
| ITK FRET-Z-Lyte SP | Test Conc(μM):ATP | 1:5 | Mean Mean | −6.9 | N |
| | Conc(μM) | | Inhib (%) | | |
| JAK1 FRET-Z-Lyte SP | Test Conc(μM):ATP | 1:75 | Mean Mean | 4.3 | N |
| | Conc(μM) | | Inhib (%) | | |
| JAK2 FRET-Z-Lyte SP | Test Conc(μM):ATP | 1:25 | Mean Mean | 4.7 | N |
| | Conc(μM) | | Inhib (%) | | |
| JAK3 FRET-Z-Lyte SP | Test Conc(μM):ATP | 1:10 | Mean Mean | −7.8 | N |
| | Conc(μM) | | Inhib (%) | | |
| LCK FRET-Z-Lyte SP | Test Conc(μM):ATP | 1:50 | Mean Mean | 39.7 | N |
| | Conc(μM) | | Inhib (%) | | |
| HCK FRET-Z-Lyte SP | Test Conc(μM):ATP | 1:25 | Mean Mean | 30.9 | N |
| | Conc(μM) | | Inhib (%) | | |
| HIPK1 (Myak) FRET-Z-Lyte SP | Test Conc(μM):ATP | 1:5 | Mean Mean | 1.2 | N |
| | Conc(μM) | | Inhib (%) | | |
| KDR (VEGFR2) FRET-Z-Lyte SP | Test Conc(μM):ATP | 1:75 | Mean Mean | 3.8 | N |
| | Conc(μM) | | Inhib (%) | | |
| HIPK2 FRET-Z-Lyte SP | Test Conc(μM):ATP | 1:10 | Mean Mean | −0.8 | N |
| | Conc(μM) | | Inhib (%) | | |
| HIPK3 (YAK1) FRET-Z-Lyte SP | Test Conc(μM):ATP | 1:25 | Mean Mean | −0.3 | N |
| | Conc(μM) | | Inhib (%) | | |
| HIPK4 FRET-Z-Lyte SP | Test Conc(μM):ATP | 1:50 | Mean Mean | 8.8 | N |
| | Conc(μM) | | Inhib (%) | | |
| IGF1R FRET-Z-Lyte SP | Test Conc(μM):ATP | 1:150 | Mean Mean | 6.5 | N |
| | Conc(μM) | | Inhib (%) | | |
| IKBKB (IKKbeta) FRET-Z-Lyte SP | Test Conc(μM):ATP | 1:5 | Mean Mean | −2.5 | N |
| | Conc(μM) | | Inhib (%) | | |
| IKBKE (IKK epsilon) Z-Lyte SP | Test Conc(μM):ATP | 1:10 | Mean Mean | −2.3 | N |
| | Conc(μM) | | Inhib (%) | | |
| INSR *Mn* FRET-Z-Lyte SP | Test Conc(μM):ATP | 1:25 | Mean Mean | 1.5 | N |
| | Conc(μM) | | Inhib (%) | | |
| INSRR (IRR) *Mn* Z-Lyte SP | Test Conc(μM):ATP | 1:50 | Mean Mean | 5.5 | N |
| | Conc(μM) | | Inhib (%) | | |
| IRAK4 *Mn* FRET-Z-Lyte SP | Test Conc(μM):ATP | 1:25 | Mean Mean | 6.8 | N |
| | Conc(μM) | | Inhib (%) | | |
| CLK2 FRET-Z-Lyte SP | Test Conc(μM):ATP | 1:25 | Mean Mean | 15.7 | N |
| | Conc(μM) | | Inhib (%) | | |
| CLK3 FRET-Z-Lyte SP | Test Conc(μM):ATP | 1:150 | Mean Mean | 5.1 | N |
| | Conc(μM) | | Inhib (%) | | |
| CSF1R (FMS) FRET-Z-Lyte SP | Test Conc(μM):ATP | 1:500 | Mean Mean | 20.0 | N |
| | Conc(μM) | | Inhib (%) | | |
| CSK FRET-Z-Lyte SP | Test Conc(μM):ATP | 1:10 | Mean Mean | 6.4 | N |
| | Conc(μM) | | Inhib (%) | | |
| CSNK1A1 FRET-Z-Lyte SP | Test Conc(μM):ATP | 1:5 | Mean Mean | 4.1 | N |
| | Conc(μM) | | Inhib (%) | | |
| ERBB2 (HER2) *Mn* Z-Lyte SP | Test Conc(μM):ATP | 1:10 | Mean Mean | 10.1 | N |
| | Conc(μM) | | Inhib (%) | | |
| CAMK1D FRET-Z-Lyte SP | Test Conc(μM):ATP | 1:25 | Mean Mean | −7.6 | N |
| | Conc(μM) | | Inhib (%) | | |
| CAMK2A FRET-Z-Lyte SP | Test Conc(μM):ATP | 1:10 | Mean Mean | −3.3 | N |
| | Conc(μM) | | Inhib (%) | | |
| CAMK2B FRET-Z-Lyte SP | Test Conc(μM):ATP | 1:75 | Mean Mean | 11.4 | N |
| | Conc(μM) | | Inhib (%) | | |
| ADRBK1 (GRK2) FRET-Z-Lyte SP | Test Conc(μM):ATP | 1:10 | Mean Mean | 1.0 | N |
| | Conc(μM) | | Inhib (%) | | |
| ADRBK2 (GRK3) FRET-Z-Lyte SP | Test Conc(μM):ATP | 1:10 | Mean Mean | −2.5 | N |
| | Conc(μM) | | Inhib (%) | | |
| AKT1 (PKB alpha) Z-Lyte SP | Test Conc(μM):ATP | 1:75 | Mean Mean | −1.3 | N |
| | Conc(μM) | | Inhib (%) | | |
| AKT2 (PKB beta) FRET-Z-Lyte SP | Test Conc(μM):ATP | 1:200 | Mean Mean | 2.3 | N |
| | Conc(μM) | | Inhib (%) | | |
| AKT3 (PKB gamma) Z-Lyte SP | Test Conc(μM):ATP | 1:100 | Mean Mean | 4.2 | N |
| | Conc(μM) | | Inhib (%) | | |
| ALK FRET-Z-Lyte SP | Test Conc(μM):ATP | 1:25 | Mean Mean | 4.2 | N |
| | Conc(μM) | | Inhib (%) | | |
| AURKA (Aurora A) Z-Lyte SP | Test Conc(μM):ATP | 1:10 | Mean Mean | 7.2 | N |
| | Conc(μM) | | Inhib (%) | | |
| AURKB (Aurora B) Z-Lyte SP | Test Conc(μM):ATP | 1:75 | Mean Mean | 2.7 | N |
| | Conc(μM) | | Inhib (%) | | |
| AURKC (Aurora C) Z-Lyte SP | Test Conc(μM):ATP | 1:25 | Mean Mean | 4.2 | N |
| | Conc(μM) | | Inhib (%) | | |
| AXL FRET-Z-Lyte SP | Test Conc(μM):ATP | 1:75 | Mean Mean | 1.1 | N |
| | Conc(μM) | | Inhib (%) | | |
| CAMK2D FRET-Z-Lyte SP | Test Conc(μM):ATP | 1:5 | Mean Mean | 0.3 | N |
| | Conc(μM) | | Inhib (%) | | |
| CAMK4 (CaMKIV) FRET-Z-Lyte SP | Test Conc(μM):ATP | 1:25 | Mean Mean | −1.8 | N |
| | Conc(μM) | | Inhib (%) | | |
| CDC42 BPA (MRCKA) Z-Lyte SP | Test Conc(μM):ATP | 1:5 | Mean Mean | −2.0 | N |
| | Conc(μM) | | Inhib (%) | | |
| CSNK1D (CK1 delta) Z-Lyte SP | Test Conc(μM):ATP | 1:5 | Mean Mean | 1.9 | N |
| | Conc(μM) | | Inhib (%) | | |
| CSNK1E (CK1 epsilon) Z-Lyte SP | Test Conc(μM):ATP | 1:5 | Mean Mean | 2.8 | N |
| | Conc(μM) | | Inhib (%) | | |
| CSNK1G1 (CK1gamma1) Z-Lyte | Test Conc(μM):ATP | 1:5 | Mean Mean | −1.9 | N |
| SP | Conc(μM) | | Inhib (%) | | |
| CSNK1G2 (CK1gamma2) Z-Lyte | Test Conc(μM):ATP | 1:5 | Mean Mean | 5.9 | N |
| SP | Conc(μM) | | Inhib (%) | | |
| DAPK3 (ZIPK) FRET-Z-Lyte SP | Test Conc(μM):ATP | 1:5 | Mean Mean | −11.8 | N |
| | Conc(μM) | | Inhib (%) | | |
| DCAMKL2 (DCK2) FRET-Z-Lyte SP | Test Conc(μM):ATP | 1:150 | Mean Mean | 4.9 | N |
| | Conc(μM) | | Inhib (%) | | |
| DNA-PK FRET-Z-Lyte SP | Test Conc(μM):ATP | 1:25 | Mean Mean | 13.9 | N |
| | Conc(μM) | | Inhib (%) | | |
| BRSK1 (SAD1) FRET-Z-Lyte SP | Test Conc(μM):ATP | 1:25 | Mean Mean | 4.7 | N |
| | Conc(μM) | | Inhib (%) | | |
| BTK FRET-Z-Lyte SP | Test Conc(μM):ATP | 1:25 | Mean Mean | 29.9 | N |
| | Conc(μM) | | Inhib (%) | | |
| BLK FRET-Z-Lyte SP | Test Conc(μM):ATP | 1:25 | Mean Mean | 9.7 | N |
| | Conc(μM) | | Inhib (%) | | |
| BMX FRET-Z-Lyte SP | Test Conc(μM):ATP | 1:100 | Mean Mean | 18.7 | N |
| | Conc(μM) | | Inhib (%) | | |
| DYRK1A FRET-Z-Lyte SP | Test Conc(μM):ATP | 1:100 | Mean Mean | −1.9 | N |
| | Conc(μM) | | Inhib (%) | | |
| DYRK1B FRET-Z-Lyte SP | Test Conc(μM):ATP | 1:75 | Mean Mean | −5.0 | N |
| | Conc(μM) | | Inhib (%) | | |
| DYRK3 FRET-Z-Lyte SP | ATP Conc(μM):Test | 1:5 | Mean Mean | −1.2 | N |
| | Conc(μM) | | Inhib (%) | | |
| CSNK1G3 (CK1gamma3) Z-Lyte | Test Conc(μM):ATP | 1:5 | Mean Mean | 0.1 | N |
| SP | Conc(μM) | | Inhib (%) | | |
| DYRK4 FRET-Z-Lyte SP | Test Conc(μM):ATP | 1:5 | Mean Mean | −4.9 | N |
| | Conc(μM) | | Inhib (%) | | |
| CSNK2A1 (CK2alpha1) Z-Lyte SP | Test Conc(μM):ATP | 1:5 | Mean Mean | 2.4 | N |
| | Conc(μM) | | Inhib (%) | | |
| CSNK2A2 (CK2alpha2) Z-Lyte SP | Test Conc(μM):ATP | 1:50 | Mean Mean | 9.5 | N |
| | Conc(μM) | | Inhib (%) | | |
| CDC42 BPB (MRCKB) Z-Lyte SP | Test Conc(μM):ATP | 1:5 | Mean Mean | −5.0 | N |
| | Conc(μM) | | Inhib (%) | | |
| CDK1/cyclin B FRET-Z-Lyte SP | Test Conc(μM):ATP | 1:25 | Mean Mean | 3.2 | N |
| | Conc(μM) | | Inhib (%) | | |
| CDK2/cyclin A FRET-Z-Lyte SP | Test Conc(μM):ATP | 1:25 | Mean Mean | 5.0 | N |
| | Conc(μM) | | Inhib (%) | | |
| CHEK1 (CHK1) FRET-Z-Lyte SP | Test Conc(μM):ATP | 1:50 | Mean Mean | 14.8 | N |
| | Conc(μM) | | Inhib (%) | | |
| CHEK2 (CHK2) FRET-Z-Lyte SP | Test Conc(μM):ATP | 1:75 | Mean Mean | 0.4 | N |
| | Conc(μM) | | Inhib (%) | | |
| FGFR2 *Mn* FRET-Z-Lyte SP | Test Conc(μM):ATP | 1:5 | Mean Mean | 6.0 | N |
| | Conc(μM) | | Inhib (%) | | |
| FGFR4 *Mn* FRET-Z-Lyte SP | Test Conc(μM):ATP | 1:150 | Mean Mean | −0.9 | N |
| | Conc(μM) | | Inhib (%) | | |
| FGR FRET-Z-Lyte SP | Test Conc(μM):ATP | 1:10 | Mean Mean | 67.3 | N |
| | Conc(μM) | | Inhib (%) | | |
| EPHA2 FRET-Z-Lyte SP | Test Conc(μM):ATP | 1:75 | Mean Mean | 30.7 | N |
| | Conc(μM) | | Inhib (%) | | |
| EPHA4 FRET-Z-Lyte SP | Test Conc(μM):ATP | 1:100 | Mean Mean | 47.0 | N |
| | Conc(μM) | | Inhib (%) | | |
| EPHA5 FRET-Z-Lyte SP | Test Conc(μM):ATP | 1:150 | Mean Mean | 65.2 | N |
| | Conc(μM) | | Inhib (%) | | |
| EPHA1 FRET-Z-Lyte SP | Test Conc(μM):ATP | 1:25 | Mean Mean | 55.3 | N |
| | Conc(μM) | | Inhib (%) | | |
| EPHB2 FRET-Z-Lyte SP | Test Conc(μM):ATP | 1:75 | Mean Mean | 58.7 | N |
| | Conc(μM) | | Inhib (%) | | |
| EPHA8 FRET-Z-Lyte SP | Test Conc(μM):ATP | 1:100 | Mean Mean | 64.7 | N |
| | Conc(μM) | | Inhib (%) | | |
| EPHB1 FRET-Z-Lyte SP | Test Conc(μM):ATP | 1:50 | Mean Mean | 47.1 | N |
| | Conc(μM) | | Inhib (%) | | |
| EEF2K FRET-Z-Lyte SP | Test Conc(μM):ATP | 1:10 | Mean Mean | 3.7 | N |
| | Conc(μM) | | Inhib (%) | | |
| ERBB4 (HER4) *Mn* Z-Lyte SP | Test Conc(μM):ATP | 1:5 | Mean Mean | 3.4 | N |
| | Conc(μM) | | Inhib (%) | | |
| FER FRET-Z-Lyte SP | Test Conc(μM):ATP | 1:25 | Mean Mean | 5.0 | N |
| | Conc(μM) | | Inhib (%) | | |
| FGFR1 *Mn* FRET-Z-Lyte SP | Test Conc(μM):ATP | 1:25 | Mean Mean | 4.3 | N |
| | Conc(μM) | | Inhib (%) | | |
| FRAP1 (mTOR) *Mn* Z-Lyte SP | Test Conc(μM):ATP | 1:10 | Mean Mean | −8.8 | N |
| | Conc(μM) | | Inhib (%) | | |
| FLT4 (VEGFR3) *Mn* Z-Lyte SP | Test Conc(μM):ATP | 1:5 | Mean Mean | 2.0 | N |
| | Conc(μM) | | Inhib (%) | | |
| FRK (PTK5) FRET-Z-Lyte SP | Test Conc(μM):ATP | 1:50 | Mean Mean | 60.1 | N |
| | Conc(μM) | | Inhib (%) | | |
| GRK4 FRET-Z-Lyte SP | Test Conc(μM):ATP | 1:10 | Mean Mean | 16.3 | N |
| | Conc(μM) | | Inhib (%) | | |
| GRK5 *Mn* FRET-Z-Lyte SP | Test Conc(μM):ATP | 1:5 | Mean Mean | 4.6 | N |
| | Conc(μM) | | Inhib (%) | | |
| FYN FRET-Z-Lyte SP | Test Conc(μM):ATP | 1:75 | Mean Mean | 43.1 | N |
| | Conc(μM) | | Inhib (%) | | |
| NEK1 FRET-Z-Lyte SP | ATP Conc(μM):Test | 1:100 | Mean Mean | 0.2 | N |
| | Conc(μM) | | Inhib (%) | | |
| NEK2 FRET-Z-Lyte SP | Test Conc(μM):ATP | 1:150 | Mean Mean | 12.8 | N |
| | Conc(μM) | | Inhib (%) | | |
| NEK4 FRET-Z-Lyte SP | Test Conc(μM):ATP | 1:50 | Mean Mean | 3.6 | N |
| | Conc(μM) | | Inhib (%) | | |
| NTRK2 (TRKB) *Mn* Z-Lyte SP | Test Conc(μM):ATP | 1:25 | Mean Mean | 0.7 | N |
| | Conc(μM) | | Inhib (%) | | |
| NTRK3 (TRKC) FRET-Z-Lyte SP | Test Conc(μM):ATP | 1:50 | Mean Mean | 2.7 | N |
| | Conc(μM) | | Inhib (%) | | |
| PAK1 FRET-Z-Lyte SP | Test Conc(μM):ATP | 1:50 | Mean Mean | 14.1 | N |
| | Conc(μM) | | Inhib (%) | | |
| PAK2 (PAK65) FRET-Z-Lyte SP | Test Conc(μM):ATP | 1:75 | Mean Mean | 12.5 | N |
| | Conc(μM) | | Inhib (%) | | |
| PAK3 FRET-Z-Lyte SP | Test Conc(μM):ATP | 1:100 | Mean Mean | 23.3 | N |
| | Conc(μM) | | Inhib (%) | | |
| PAK4 FRET-Z-Lyte SP | Test Conc(μM):ATP | 1:5 | Mean Mean | 7.4 | N |
| | Conc(μM) | | Inhib (%) | | |
| NTRK1 (TRKA) FRET-Z-Lyte SP | Test Conc(μM):ATP | 1:400 | Mean Mean | 20.1 | N |
| | Conc(μM) | | Inhib (%) | | |
| MARK2 FRET-Z-Lyte SP | Test Conc(μM):ATP | 1:10 | Mean Mean | 3.8 | N |
| | Conc(μM) | | Inhib (%) | | |
| MARK3 FRET-Z-Lyte SP | Test Conc(μM):ATP | 1:5 | Mean Mean | 4.7 | N |
| | Conc(μM) | | Inhib (%) | | |
| MARK4 FRET-Z-Lyte SP | Test Conc(μM):ATP | 1:10 | Mean Mean | 1.4 | N |
| | Conc(μM) | | Inhib (%) | | |
| MATK (HYL) FRET-Z-Lyte SP | Test Conc(μM):ATP | 0.25 | Mean Mean | 1.8 | N |
| | Conc(μM) | | Inhib (%) | | |
| MELK FRET-Z-Lyte SP | Test Conc(μM):ATP | 1:25 | Mean Mean | 6.0 | N |
| | Conc(μM) | | Inhib (%) | | |
| MERTK (cMER) *Mn* Z-Lyte SP | Test Conc(μM):ATP | 1:10 | Mean Mean | 7.3 | N |
| | Conc(μM) | | Inhib (%) | | |
| NEK6 FRET-Z-Lyte SP | Test Conc(μM):ATP | 1:100 | Mean Mean | 8.2 | N |
| | Conc(μM) | | Inhib (%) | | |
| NEK7 FRET-Z-Lyte SP | Test Conc(μM):ATP | 1:50 | Mean Mean | 6.9 | N |
| | Conc(μM) | | Inhib (%) | | |
| NEK9 *Mn* FRET-Z-Lyte SP | Test Conc(μM):ATP | 1:100 | Mean Mean | −4.1 | N |
| | Conc(μM) | | Inhib (%) | | |
| MAPK13 (p38 delta) Z-Lyte SP | Test Conc(μM):ATP | 1:10 | Mean Mean | 3.7 | N |
| | Conc(μM) | | Inhib (%) | | |
| MAPK3 (ERK1) FRET-Z-Lyte SP | Test Conc(μM):ATP | 1:50 | Mean Mean | 1.1 | N |
| | Conc(μM) | | Inhib (%) | | |
| MAPK8 (JNK1) Cascade Z-Lyte SP | Test Conc(μM):ATP | 1:100 | Mean Mean | 16.9 | N |
| | Conc(μM) | | Inhib (%) | | |
| MAPK9 (JNK2) Cascade Z-Lyte SP | Test Conc(μM):ATP | 1:100 | Mean Mean | 12.1 | N |
| | Conc(μM) | | Inhib (%) | | |
| LTK (TYK1) FRET-Z-Lyte SP | Test Conc(μM):ATP | 1:75 | Mean Mean | −3.9 | N |
| | Conc(μM) | | Inhib (%) | | |
| MAP2K1 (MEK1)Cascade Z-Lyte | Test Conc(μM):ATP | 1:100 | Mean Mean | 17.3 | N |
| SP | Conc(μM) | | Inhib (%) | | |
| MAP2K2 (MEK2)Cascade Z-Lyte | Test Conc(μM):ATP | 1:100 | Mean Mean | 10.4 | N |
| SP | Conc(μM) | | Inhib (%) | | |
| MAP2K6 (MKK6)Cascade Z-Lyte | Test Conc(μM):ATP | 1:100 | Mean Mean | −5.7 | N |
| SP | Conc(μM) | | Inhib (%) | | |
| MAP3K8 (COT) Cascade Z-Lyte SP | Test Conc(μM):ATP | 1:100 | Mean Mean | 21.2 | N |
| | Conc(μM) | | Inhib (%) | | |
| MAP3K9 (MLK1) FRET-Z-Lyte SP | Test Conc(μM):ATP | 1:75 | Mean Mean | 3.4 | N |
| | Conc(μM) | | Inhib (%) | | |
| MAP4K2 (GCK) FRET-Z-Lyte SP | Test Conc(μM):ATP | 1:100 | Mean Mean | 18.4 | N |
| | Conc(μM) | | Inhib (%) | | |
| MAP4K4 (HGK) FRET-Z-Lyte SP | Test Conc(μM):ATP | 1:10 | Mean Mean | 12.0 | N |
| | Conc(μM) | | Inhib (%) | | |
| MAP4K5 (KHS1) FRET-Z-Lyte SP | Test Conc(μM):ATP | 1:50 | Mean Mean | 38.0 | N |
| | Conc(μM) | | Inhib (%) | | |
| MAPK1 (ERK2) FRET-Z-Lyte SP | Test Conc(μM):ATP | 1:100 | Mean Mean | 7.5 | N |
| | Conc(μM) | | Inhib (%) | | |
| MAPKAPK2 FRET-Z-Lyte SP | Test Conc(μM):ATP | 1:5 | Mean Mean | 9.2 | N |
| | Conc(μM) | | Inhib (%) | | |
| MAPKAPK3 FRET-Z-Lyte SP | Test Conc(μM):ATP | 1:200 | Mean Mean | −1.3 | N |
| | Conc(μM) | | Inhib (%) | | |
| MAPKAPK5 (PRAK) FRET-Z-Lyte | Test Conc(μM):ATP | 1:10 | Mean Mean | 1.9 | N |
| SP | Conc(μM) | | Inhib (%) | | |
| MARK1 (MARK) FRET-Z-Lyte SP | Test Conc(μM):ATP | 1:5 | Mean Mean | 5.7 | N |
| | Conc(μM) | | Inhib (%) | | |
| MAPK11 (p38 beta) Z-Lyte SP | Test Conc(μM):ATP | 1:50 | Mean Mean | 21.2 | N |
| | Conc(μM) | | Inhib (%) | | |
| MAPK12 (p38 gamma) Z-Lyte SP | Test Conc(μM):ATP | 1:10 | Mean Mean | 4.1 | N |
| | Conc(μM) | | Inhib (%) | | |
| MAPK10 (JNK3)Cascade Z-Lyte SP | Test Conc(μM):ATP | 1:100 | Mean Mean | 1.1 | N |
| | Conc(μM) | | Inhib (%) | | |
| SYK FRET-Z-Lyte SP | Test Conc(μM):ATP | 1:25 | Mean Mean | 2.5 | N |
| | Conc(μM) | | Inhib (%) | | |
| TAOK2 (TAO1) FRET-Z-Lyte SP | Test Conc(μM):ATP | 0.25 | Mean Mean | 13.7 | N |
| | Conc(μM) | | Inhib (%) | | |
| TBK1 FRET-Z-Lyte SP | Test Conc(μM):ATP | 1:25 | Mean Mean | 4.2 | N |
| | Conc(μM) | | Inhib (%) | | |
| TEK (Tie2) *Mn* FRET-Z-Lyte SP | Test Conc(μM):ATP | 1:10 | Mean Mean | −7.9 | N |
| | Conc(μM) | | Inhib (%) | | |
| TXK FRET-Z-Lyte SP | Test Conc(μM):ATP | 1:100 | Mean Mean | 44.8 | N |
| | Conc(μM) | | Inhib (%) | | |
| TYK2 FRET-Z-Lyte SP | Test Conc(μM):ATP | 1:25 | Mean Mean | 8.4 | N |
| | Conc(μM) | | Inhib (%) | | |
| TYRO3 (RSE) FRET-Z-Lyte SP | Test Conc(μM):ATP | 1:25 | Mean Mean | 1.4 | N |
| | Conc(μM) | | Inhib (%) | | |
| PDGFRB *Mn* FRET-Z-Lyte SP | Test Conc(μM):ATP | 1:100 | Mean Mean | 3.9 | N |
| | Conc(μM) | | Inhib (%) | | |
| PHKG1 FRET-Z-Lyte SP | Test Conc(μM):ATP | 1:75 | Mean Mean | −6.4 | N |
| | Conc(μM) | | Inhib (%) | | |
| PHKG2 FRET-Z-Lyte SP | Test Conc(μM):ATP | 1:10 | Mean Mean | 4.0 | N |
| | Conc(μM) | | Inhib (%) | | |
| PIM1 FRET-Z-Lyte SP | Test Conc(μM):ATP | 1:400 | Mean Mean | 3.8 | N |
| | Conc(μM) | | Inhib (%) | | |
| PIM2 FRET-Z-Lyte SP | Test Conc(μM):ATP | 1:5 | Mean Mean | 3.7 | N |
| | Conc(μM) | | Inhib (%) | | |
| PKN1 (PRK1) FRET-Z-Lyte SP | Test Conc(μM):ATP | 1:50 | Mean Mean | 0.4 | N |
| | Conc(μM) | | Inhib (%) | | |
| PLK1 FRET-Z-Lyte SP | Test Conc(μM):ATP | 1:10 | Mean Mean | 9.6 | N |
| | Conc(μM) | | Inhib (%) | | |
| PLK2 FRET-Z-Lyte SP | Test Conc(μM):ATP | 1:25 | Mean Mean | 14.0 | N |
| | Conc(μM) | | Inhib (%) | | |
| PRKG1 FRET-Z-Lyte SP | Test Conc(μM):ATP | 1:25 | Mean Mean | 0.3 | N |
| | Conc(μM) | | Inhib (%) | | |
| PRKG2 (PKG2) FRET-Z-Lyte SP | Test Conc(μM):ATP | 1:150 | Mean Mean | 0.2 | N |
| | Conc(μM) | | Inhib (%) | | |
| PRKX FRET-Z-Lyte SP | Test Conc(μM):ATP | 1:10 | Mean Mean | 1.8 | N |
| | Conc(μM) | | Inhib (%) | | |
| PTK2 (FAK) FRET-Z-Lyte SP | Test Conc(μM):ATP | 1:50 | Mean Mean | 5.7 | N |
| | Conc(μM) | | Inhib (%) | | |
| RPS6KA5 (MSK1) FRET-Z-Lyte SP | ATP Conc(μM):Test | 1:50 | Mean Mean | 10.0 | N |
| | Conc(μM) | | Inhib (%) | | |
| RPS6KA6 (RSK4) FRET-Z-Lyte SP | Test Conc(μM):ATP | 1:25 | Mean Mean | −0.7 | N |
| | Conc(μM) | | Inhib (%) | | |
| RPS6KB1 (p70S6K) Z-Lyte SP | Test Conc(μM):ATP | 1:10 | Mean Mean | 1.0 | N |
| | Conc(μM) | | Inhib (%) | | |
| SGK (SGK1) FRET-Z-Lyte SP | Test Conc(μM):ATP | 1:25 | Mean Mean | −1.4 | N |
| | Conc(μM) | | Inhib (%) | | |
| SRMS (Srm) FRET-Z-Lyte SP | Test Conc(μM):ATP | 1:150 | Mean Mean | 1.5 | N |
| | Conc(μM) | | Inhib (%) | | |
| SRPK1 FRET-Z-Lyte SP | Test Conc(μM):ATP | 1:25 | Mean Mean | 2.9 | N |
| | Conc(μM) | | Inhib (%) | | |
| SRPK2 FRET-Z-Lyte SP | Test Conc(μM):ATP | 1:25 | Mean Mean | 1.9 | N |
| | Conc(μM) | | Inhib (%) | | |
| STK22B (TSSK2) FRET-Z-Lyte SP | Test Conc(μM):ATP | 1:5 | Mean Mean | −0.9 | N |
| | Conc(μM) | | Inhib (%) | | |
| STK22D (TSSK1) FRET-Z-Lyte SP | Test Conc(μM):ATP | 1:10 | Mean Mean | 12.2 | N |
| | Conc(μM) | | Inhib (%) | | |
| STK23 (MSSK1) FRET-Z-Lyte SP | Test Conc(μM):ATP | 1:75 | Mean Mean | −4.8 | N |
| | Conc(μM) | | Inhib (%) | | |
| STK24 (MST3) FRET-Z-Lyte SP | ATP Conc(μM):Test | 1:50 | Mean Mean | 4.9 | N |
| | Conc(μM) | | Inhib (%) | | |
| SGK2 FRET-Z-Lyte SP | Test Conc(μM):ATP | 1:50 | Mean Mean | −1.3 | N |
| | Conc(μM) | | Inhib (%) | | |
| SGKL (SGK3) FRET-Z-Lyte SP | Test Conc(μM):ATP | 1:10 | Mean Mean | 4.7 | N |
| | Conc(μM) | | Inhib (%) | | |
| SNF1LK2 FRET-Z-Lyte SP | Test Conc(μM):ATP | 1:50 | Mean Mean | 10.7 | N |
| | Conc(μM) | | Inhib (%) | | |
| PTK2B (FAK2) *Mn* Z-Lyte SP | Test Conc(μM):ATP | 1:5 | Mean Mean | −2.5 | N |
| | Conc(μM) | | Inhib (%) | | |
| PTK6 (Brk) *Mn* FRET-Z-Lyte SP | Test Conc(μM):ATP | 1:75 | Mean Mean | 32.3 | N |
| | Conc(μM) | | Inhib (%) | | |
| ROCK1 FRET-Z-Lyte SP | Test Conc(μM):ATP | 1:5 | Mean Mean | 1.6 | N |
| | Conc(μM) | | Inhib (%) | | |
| ROCK2 FRET-Z-Lyte SP | Test Conc(μM):ATP | 1:50 | Mean Mean | 1.6 | N |
| | Conc(μM) | | Inhib (%) | | |
| ROS1 FRET-Z-Lyte SP | Test Conc(μM):ATP | 1:50 | Mean Mean | 8.9 | N |
| | Conc(μM) | | Inhib (%) | | |
| RPS6KA1 (RSK1) FRET-Z-Lyte SP | Test Conc(μM):ATP | 1:5 | Mean Mean | 5.3 | N |
| | Conc(μM) | | Inhib (%) | | |
| RPS6KA2 (RSK3) FRET-Z-Lyte SP | Test Conc(μM):ATP | 1:10 | Mean Mean | 0.2 | N |
| | Conc(μM) | | Inhib (%) | | |
| RPS6KA3 (RSK2) FRET-Z-Lyte SP | Test Conc(μM):ATP | 1:10 | Mean Mean | −3.2 | N |
| | Conc(μM) | | Inhib (%) | | |
| RPS6KA4 (MSK2) FRET-Z-Lyte SP | Test Conc(μM):ATP | 1:10 | Mean Mean | 5.3 | N |
| | Conc(μM) | | Inhib (%) | | |
| ZAP70 *Mn* FRET-Z-Lyte SP | Test Conc(μM):ATP | 1:5 | Mean Mean | 0.0 | N |
| | Conc(μM) | | Inhib (%) | | |
| STK3 (MST2) FRET-Z-Lyte SP | Test Conc(μM):ATP | 1:50 | Mean Mean | 1.1 | N |
| | Conc(μM) | | Inhib (%) | | |
| STK4 (MST1) FRET-Z-Lyte SP | Test Conc(μM):ATP | 1:50 | Mean Mean | 8.7 | N |
| | Conc(μM) | | Inhib (%) | | |
| STK25 (YSK1) FRET-Z-Lyte SP | Test Conc(μM):ATP | 1:75 | Mean Mean | 2.0 | N |
| | Conc(μM) | | Inhib (%) | | |
| YES1 FRET-Z-Lyte SP | Test Conc(μM):ATP | 1:25 | Mean Mean | 56.6 | N |
| | Conc(μM) | | Inhib (%) | | |
| PRKCI (PKC iota) Z-Lyte SP | Test Conc(μM):ATP | 1:25 | Mean Mean | 9.7 | N |
| | Conc(μM) | | Inhib (%) | | |
| PRKCN (PKD3) FRET-Z-Lyte SP | Test Conc(μM):ATP | 1:25 | Mean Mean | 12.4 | N |
| | Conc(μM) | | Inhib (%) | | |
| PRKCQ (PKC theta) Z-Lyte SP | Test Conc(μM):ATP | 1:100 | Mean Mean | 5.9 | N |
| | Conc(μM) | | Inhib (%) | | |
| PRKCD (PKC delta) Z-Lyte SP | Test Conc(μM):ATP | 1:25 | Mean Mean | 1.0 | N |
| | Conc(μM) | | Inhib (%) | | |
| PRKCE (PKC epsilon) Z-Lyte SP | Test Conc(μM):ATP | 1:25 | Mean Mean | 10.0 | N |
| | Conc(μM) | | Inhib (%) | | |
| PRKCG (PKC gamma) Z-Lyte SP | Test Conc(μM):ATP | 1:25 | Mean Mean | −13.8 | N |
| | Conc(μM) | | Inhib (%) | | |
| PAK6 FRET-Z-Lyte SP | Test Conc(μM):ATP | 1:10 | Mean Mean | 16.2 | N |
| | Conc(μM) | | Inhib (%) | | |
| PAK7 (KIAA1264) FRET-Z-Lyte SP | Test Conc(μM):ATP | 1:5 | Mean Mean | 4.7 | N |
| | Conc(μM) | | Inhib (%) | | |
| PASK FRET-Z-Lyte SP | Test Conc(μM):ATP | 1:50 | Mean Mean | 3.8 | N |
| | Conc(μM) | | Inhib (%) | | |
| PRKD1 (PKC mu) Z-Lyte SP | Test Conc(μM):ATP | 1:10 | Mean Mean | 11.3 | N |
| | Conc(μM) | | Inhib (%) | | |
| PRKCZ (PKC zeta) Z-Lyte SP | Test Conc(μM):ATP | 1:5 | Mean Mean | −22.4 | N |
| | Conc(μM) | | Inhib (%) | | |
| PRKD2 (PKD2) Z-Lyte SP | Test Conc(μM):ATP | 1:25 | Mean Mean | 21.2 | N |
| | Conc(μM) | | Inhib (%) | | |
| MKNK1 (MNK1) *Mn* Z-Lyte SP | Test Conc(μM):ATP | 1:100 | Mean Mean | 1.5 | N |
| | Conc(μM) | | Inhib (%) | | |
| MINK1 FRET-Z-Lyte SP | Test Conc(μM):ATP | 1:25 | Mean Mean | 19.9 | N |
| | Conc(μM) | | Inhib (%) | | |
| MST1R (RON) FRET-Z-Lyte SP | Test Conc(μM):ATP | 1:10 | Mean Mean | 5.3 | N |
| | Conc(μM) | | Inhib (%) | | |
| MST4 FRET-Z-Lyte SP | Test Conc(μM):ATP | 1:25 | Mean Mean | −9.9 | N |
| | Conc(μM) | | Inhib (%) | | |
| MUSK *Mn* FRET-Z-Lyte SP | Test Conc(μM):ATP | 1:50 | Mean Mean | 17.2 | N |
| | Conc(μM) | | Inhib (%) | | |
| PLK3 FRET-Z-Lyte SP | Test Conc(μM):ATP | 1:50 | Mean Mean | −4.9 | N |
| | Conc(μM) | | Inhib (%) | | |
| PRKACA (PKA) FRET-Z-Lyte SP | Test Conc(μM):ATP | 1:5 | Mean Mean | −4.7 | N |
| | Conc(μM) | | Inhib (%) | | |
| PRKCA (PKC alpha) Z-Lyte SP | Test Conc(μM):ATP | 1:25 | Mean Mean | −2.0 | N |
| | Conc(μM) | | Inhib (%) | | |
| PRKCB1 (PKC beta I) Z-Lyte SP | Test Conc(μM):ATP | 1:200 | Mean Mean | −7.3 | N |
| | Conc(μM) | | Inhib (%) | | |
| PRKCH (PKC eta) Z-Lyte SP | Test Conc(μM):ATP | 1:25 | Mean Mean | 0.8 | N |
| | Conc(μM) | | Inhib (%) | | |
| EGFR (ErbB1) *Mn* Z-Lyte SP | Test Conc(μM):ATP | 1:10 | Mean Mean | −1.9 | N |
| | Conc(μM) | | Inhib (%) | | |
| ABL1 Hu Phos FRET-Z-Lyte SP | Test Conc(μM):ATP | 1:10 | Mean Mean | 85.2 | A |
| | Conc(μM) | | Inhib (%) | | |
| AMPK A2/B1/G1 FRET-Z-Lyte SP | Test Conc(μM):ATP | 1:150 | Mean Mean | 2.9 | N |
| | Conc(μM) | | Inhib (%) | | |
| bRAF Cascade FRET-Z-Lyte SP | Test Conc(μM):ATP | 1:100 | Mean Mean | 16.7 | N |
| | Conc(μM) | | Inhib (%) | | |
| DDR2 Hu Bind FRET-Lantha SP | Test Conc(μM) | 1 | Mean Mean | 1.6 | N |
| | | | Inhib (%) | | |
| FES (FPS) FRET-Z-Lyte SP | Test Conc(μM):ATP | 1:25 | Mean Mean | 6.3 | N |
| | Conc(μM) | | Inhib (%) | | |
| FGFR3 *Mn* FRET-Z-Lyte SP | Test Conc(μM):ATP | 1:75 | Mean Mean | 7.7 | N |
| | Conc(μM) | | Inhib (%) | | |
| FLT1 (VEGFR1) *Mn* Z-Lyte SP | Test Conc(μM):ATP | 1:150 | Mean Mean | 2.7 | N |
| | Conc(μM) | | Inhib (%) | | |
| FLT3 Hu Phos FRET-Z-Lyte SP | Test Conc(μM):ATP | 1:500 | Mean Mean | 8.9 | N |
| | Conc(μM) | | Inhib (%) | | |
| KIT *Mn* FRET-Z-Lyte SP | Test Conc(μM):ATP | 0.25 | Mean Mean | 8.8 | N |
| | Conc(μM) | | Inhib (%) | | |
| LYN A Hu Phos FRET-Z-Lyte SP | Test Conc(μM):ATP | 1:25 | Mean Mean | 49.5 | N |
| | Conc(μM) | | Inhib (%) | | |
| MET (cMet) FRET-Z-Lyte SP | Test Conc(μM):ATP | 1:50 | Mean Mean | 2.5 | N |
| | Conc(μM) | | Inhib (%) | | |
| PDK1 Cascade FRET-Z-Lyte SP | Test Conc(μM):ATP | 1:100 | Mean Mean | 9.4 | N |
| | Conc(μM) | | Inhib (%) | | |
| RET Hu Phos FRET-Z-Lyte SP | Test Conc(μM):ATP | 1:10 | Mean Mean | 31.0 | N |
| | Conc(μM) | | Inhib (%) | | |
| SRC Hu Phos FRET-Z-Lyte SP | Test Conc(μM):ATP | 1:50 | Mean Mean | 54.4 | N |
| | Conc(μM) | | Inhib (%) | | |
| TGFBR1 (ALK5) FRET-Lantha SP | Test Conc(μM) | 1 | Mean Mean | 99.6 | A |
| | | | Inhib (%) | | |
| MAPK14 (p38a)Cascade Z-Lyte SP | Test Conc(μM):ATP | 1:100 | Mean Mean | 13.5 | N |
| | Conc(μM) | | Inhib (%) | | |
| MAPK14 (p38a) Direct Z-Lyte SP | Test Conc(μM):ATP | 1:500 | Mean Mean | 19.9 | N |
| | Conc(μM) | | Inhib (%) | | |
| PDK1 (Direct) FRET-Z-lyte SP | Test Conc(μM):ATP | 1:25 | Mean Mean | 6.9 | N |
| | Conc(μM) | | Inhib (%) | | |
| ACVRL1 (ALK1) FRET-Lantha SP | Test Conc(μM) | 1 | Mean Mean | 77.2 | A |
| | | | Inhib (%) | | |
| CLK1 FRET-Z-Lyte SP | Test Conc(μM):ATP | 1:25 | Mean Mean | 5.8 | N |
| | Conc(μM) | | Inhib (%) | | |
| CLK4 FRET-Lantha SP | Test Conc(μM) | 1 | Mean Mean | 0.4 | N |
| | | | Inhib (%) | | |
| MAP4K3 (GLK) FRET-Lantha SP | Test Conc(μM) | 1 | Mean Mean | 14.0 | N |
| | | | Inhib (%) | | |
| ACVR1 (ALK2) FRET-Lantha SP | Test Conc(μM) | 1 | Mean Mean | 88.2 | A |
| | | | Inhib (%) | | |
| AMPK A1/B1/G1 FRET-Z-lyte SP | Test Conc(μM):ATP | 1:50 | Mean Mean | −0.6 | N |
| | Conc(μM) | | Inhib (%) | | |
| AMPK (A1/B1/G2) FRET-Lantha SP | Test Conc(μM) | 1 | Mean Mean | 1.3 | N |
| | | | Inhib (%) | | |
| AMPK (A1/B1/G3) FRET-Lantha SP | Test Conc(μM) | 1 | Mean Mean | −1.2 | N |
| | | | Inhib (%) | | |
| AMPK (A1/B2/G1) FRET-Lantha SP | Test Conc(μM) | 1 | Mean Mean | 2.3 | N |
| | | | Inhib (%) | | |
| AMPK (A2/B2/G1) FRET-Lantha SP | Test Conc(μM) | 1 | Mean Mean | −1.2 | N |
| | | | Inhib (%) | | |
| AMPK (A2/B2/G2) FRET-Lantha SP | Test Conc(μM) | 1 | Mean Mean | 1.4 | N |
| | | | Inhib (%) | | |
| bRAF FRET-Lantha SP | Test Conc(μM) | 1 | Mean Mean | 10.4 | N |
| | | | Inhib (%) | | |
| CDK1/cyclin A2 FRET-Lantha SP | Test Conc(μM) | 1 | Mean Mean | 25.4 | N |
| | | | Inhib (%) | | |
| CDK11 (Inactive) FRET-Lantha SP | Test Conc(μM) | 1 | Mean Mean | 1.0 | N |
| | | | Inhib (%) | | |
| CDK14(PFTK1)/cyclinY Lantha SP | Test Conc(μM) | 1 | Mean Mean | 11.7 | N |
| | | | Inhib (%) | | |
| CDK2/cyclin A1 FRET-Lantha SP | Test Conc(μM) | 1 | Mean Mean | 4.9 | N |
| | | | Inhib (%) | | |
| CDK3/cyclin E1 FRET-Lantha SP | Test Conc(μM) | 1 | Mean Mean | 9.6 | N |
| | | | Inhib (%) | | |
| CDK5/p25 FRET-Z-Lyte SP | Test Conc(μM):ATP | 1:10 | Mean Mean | 8.0 | N |
| | Conc(μM) | | Inhib (%) | | |
| CDK5/p35 FRET-Z-Lyte SP | Test Conc(μM):ATP | 1:10 | Mean Mean | 2.5 | N |
| | Conc(μM) | | Inhib (%) | | |
| CDK9/cyclin K FRET-Lantha SP | Test Conc(μM) | 1 | Mean Mean | 2.6 | N |
| | | | Inhib (%) | | |
| FYN A FRET-Lantha SP | Test Conc(μM) | 1 | Mean Mean | 27.5 | N |
| | | | Inhib (%) | | |
| JAK2 JH1 JH2 FRET-Z-Lyte SP | ATP Conc(μM):Test | 1:50 | Mean Mean | −3.9 | N |
| | Conc(μM) | | Inhib (%) | | |
| LRRK2 FL FRET-Adapta SP | Test Conc(μM):ATP | 1:50 | Mean Mean | 3.0 | N |
| | Conc(μM) | | Inhib (%) | | |
| LYN B FRET-Z-Lyte SP | Test Conc(μM):ATP | 1:25 | Mean Mean | 42.0 | N |
| | Conc(μM) | | Inhib (%) | | |
| MYLK2 FRET-Z-lyte SP | Test Conc(μM):ATP | 0.25 | Mean Mean | −7.8 | N |
| | Conc(μM) | | Inhib (%) | | |
| PDGFRA *Mn* FRET-Z-Lyte SP | Test Conc(μM):ATP | 1:10 | Mean Mean | 7.6 | N |
| | Conc(μM) | | Inhib (%) | | |
| PRKCB2 (PKC Beta II) Z-Lyte SP | Test Conc(μM):ATP | 1:200 | Mean Mean | −1.0 | N |
| | Conc(μM) | | Inhib (%) | | |
| CDK2/Cyclin E1 FRET-Lantha SP | Test Conc(μM) | 1 | Mean Mean | 10.6 | N |
| | | | Inhib (%) | | |
| CDK2/Cyclin O FRET-Lantha SP | Test Conc(μM) | 1 | Mean Mean | 3.0 | N |
| | | | Inhib (%) | | |
| SIK1 FRET-Lantha SP | Test Conc(μM) | 1 | Mean Mean | 1.8 | N |
| | | | Inhib (%) | | |
| STK38 FRET-Lantha SP | Test Conc(μM) | 1 | Mean Mean | 3.1 | N |
| | | | Inhib (%) | | |
| MYLK3 Hu Bind FRET-Lantha SP | Test Conc(μM) | 1 | Mean Mean | 0.8 | N |
| | | | Inhib (%) | | |
| SLK Hu Bind FRET-Lantha SP | Test Conc(μM) | 1 | Mean Mean | 3.0 | N |
| | | | Inhib (%) | | |
| AMPK A1/B2/G3 FRET-Z-Lyte SP | Test Conc(μM):ATP | 1:10 | Mean Mean | −2.4 | N |
| | Conc(μM) | | Inhib (%) | | |
| AMPK A2/B1/G2 FRET-Z-Lyte SP | Test Conc(μM):ATP | 1:10 | Mean Mean | −6.7 | N |
| | Conc(μM) | | Inhib (%) | | |
| AMPK A2/B1/G3 FRET-Z-Lyte SP | Test Conc(μM):ATP | 1:25 | Mean Mean | −5.2 | N |
| | Conc(μM) | | Inhib (%) | | |
| AMPK A2/B2/G3 FRET-Z-Lyte SP | Test Conc(μM):ATP | 1:50 | Mean Mean | −4.3 | N |
| | Conc(μM) | | Inhib (%) | | |
| MAPK7 (ERK5) FRET-Z-Lyte SP | Test Conc(μM):ATP | 1:10 | Mean Mean | 6.6 | N |
| | Conc(μM) | | Inhib (%) | | |
| CAMK1G FRET-Z-Lyte SP | Test Conc(μM):ATP | 0.25 | Mean Mean | −3.7 | N |
| | Conc(μM) | | Inhib (%) | | |
| CDC42 BPG FRET-Z-Lyte SP | Test Conc(μM):ATP | 1:5 | Mean Mean | −5.0 | N |
| | Conc(μM) | | Inhib (%) | | |
| CDKL5 FRET-Z-Lyte SP | Test Conc(μM):ATP | 1:500 | Mean Mean | 9.8 | N |
| | Conc(μM) | | Inhib (%) | | |
| CSNK1A1L FRET-Z-Lyte SP | Test Conc(μM):ATP | 1:5 | Mean Mean | 0.5 | N |
| | Conc(μM) | | Inhib (%) | | |
| DCAMKL1 (DCLK1) FRET-Z-Lyte | Test Conc(μM):ATP | 0.25 | Mean Mean | 11.8 | N |
| SP | Conc(μM) | | Inhib (%) | | |
| CDK17/cyclin Y FRET-Z-Lyte SP | Test Conc(μM):ATP | 1:50 | Mean Mean | 9.6 | N |
| | Conc(μM) | | Inhib (%) | | |
| CDK18/cyclin Y FRET-Z-Lyte SP | Test Conc(μM):ATP | 1:75 | Mean Mean | 1.2 | N |
| | Conc(μM) | | Inhib (%) | | |
| MAP3K19 (YSK4) FRET-Z-Lyte SP | Test Conc(μM):ATP | 1:5 | Mean Mean | −3.1 | N |
| | Conc(μM) | | Inhib (%) | | |
| NIM1K FRET-Z-Lyte SP | Test Conc(μM):ATP | 1:10 | Mean Mean | −0.4 | N |
| | Conc(μM) | | Inhib (%) | | |
| PIM3 FRET-Z-Lyte SP | Test Conc(μM):ATP | 1:1000 | Mean Mean | −2.6 | N |
| | Conc(μM) | | Inhib (%) | | |
| SBK1 FRET-Z-Lyte SP | Test Conc(μM):ATP | 1:5 | Mean Mean | −6.2 | N |
| | Conc(μM) | | Inhib (%) | | |
| TNK1 FRET-Z-Lyte SP | Test Conc(μM):ATP | 1:200 | Mean Mean | −4.5 | N |
| | Conc(μM) | | Inhib (%) | | |
| PI4K2A *Mn* FRET-Adapta SP | Test Conc(μM):ATP | 1:10 | Mean Mean | −2.9 | N |
| | Conc(μM) | | Inhib (%) | | |
| PI4K2B *Mn* FRET-Adapta SP | Test Conc(μM):ATP | 1:10 | Mean Mean | 1.8 | N |
| | Conc(μM) | | Inhib (%) | | |
| PIK3C2G FRET-Adapta SP | Test Conc(μM):ATP | 1:10 | Mean Mean | 0.4 | N |
| | Conc(μM) | | Inhib (%) | | |
| PIK3CA/PIK3R3 FRET-Adapta SP | Test Conc(μM):ATP | 1:10 | Mean Mean | 3.9 | N |
| | Conc(μM) | | Inhib (%) | | |
| PIK3CB/PIK3R2 FRET-Adapta SP | Test Conc(μM):ATP | 1:10 | Mean Mean | −4.7 | N |
| | Conc(μM) | | Inhib (%) | | |
| AAK1 FRET-Lantha SP | Test Conc(μM) | 1 | Mean Mean | 2.4 | N |
| | | | Inhib (%) | | |
| ADCK3 FRET-Lantha SP | Test Conc(μM) | 1 | Mean Mean | −3.0 | N |
| | | | Inhib (%) | | |
| ERN1 FRET-Lantha SP | Test Conc(μM) | 1 | Mean Mean | 1.0 | N |
| | | | Inhib (%) | | |
| GAK FRET-Lantha SP | Test Conc(μM) | 1 | Mean Mean | 39.3 | N |
| | | | Inhib (%) | | |
| HUNK FRET-Lantha SP | Test Conc(μM) | 1 | Mean Mean | 11.5 | N |
| | | | Inhib (%) | | |
| IRAK3 FRET-Lantha SP | Test Conc(μM) | 1 | Mean Mean | 5.4 | N |
| | | | Inhib (%) | | |
| MAP2K4 (MEK4) FRET-Lantha SP | Test Conc(μM) | 1 | Mean Mean | 14.1 | N |
| | | | Inhib (%) | | |
| MAP2K5 (MEK5) FRET-Lantha SP | Test Conc(μM) | 1 | Mean Mean | 3.4 | N |
| | | | Inhib (%) | | |
| MLK4 FRET-Lantha SP | Test Conc(μM) | 1 | Mean Mean | 1.9 | N |
| | | | Inhib (%) | | |
| MYLK4 FRET-Lantha SP | Test Conc(μM) | 1 | Mean Mean | 14.6 | N |
| | | | Inhib (%) | | |
| MYO3A (MYO3 alpha) Lantha SP | Test Conc(μM) | 1 | Mean Mean | 1.6 | N |
| | | | Inhib (%) | | |
| PKMYT1 FRET-Lantha SP | Test Conc(μM) | 1 | Mean Mean | −14.5 | N |
| | | | Inhib (%) | | |
| TESK1 FRET-Lantha SP | Test Conc(μM) | 1 | Mean Mean | 15.5 | N |
| | | | Inhib (%) | | |
| VRK2 FRET-Lantha SP | Test Conc(μM) | 1 | Mean Mean | −16.6 | N |
| | | | Inhib (%) | | |
| KSR2 FRET-Z-Lyte SP | Test Conc(μM):ATP | 1:10 | Mean Mean | 1.6 | N |
| | Conc(μM) | | Inhib (%) | | |
| PEAK1 FRET-Z-Lyte SP | Test Conc(μM):ATP | 1:50 | Mean Mean | 55.2 | N |
| | Conc(μM) | | Inhib (%) | | |
| RPS6KB2 FRET-Z-Lyte SP | Test Conc(μM):ATP | 1:50 | Mean Mean | 2.1 | N |
| | Conc(μM) | | Inhib (%) | | |
| ERN2 FRET-Lantha SP | Test Conc(μM) | 1 | Mean Mean | 7.1 | N |
| | | | Inhib (%) | | |
| MASTL FRET-Lantha SP | Test Conc(μM) | 1 | Mean Mean | 13.5 | N |
| | | | Inhib (%) | | |
| NEK8 FRET-Lantha SP | Test Conc(μM) | 1 | Mean Mean | −7.5 | N |
| | | | Inhib (%) | | |
| AMPK A1/B2/G2 FRET-Z-Lyte SP | Test Conc(μM):ATP | 1:10 | Mean Mean | −0.5 | N |
| | Conc(μM) | | Inhib (%) | | |
| CDK6 Cyclin D1 FRET-Adapta SP | Test Conc(μM):ATP | 1:10 | Mean Mean | 6.7 | N |
| | Conc(μM) | | Inhib (%) | | |
| PIP4K2A FRET-Adapta SP | Test Conc(μM):ATP | 1:10 | Mean Mean | −8.6 | N |
| | Conc(μM) | | Inhib (%) | | |
| PIP5K1A FRET-Adapta SP | Test Conc(μM):ATP | 1:10 | Mean Mean | 2.4 | N |
| | Conc(μM) | | Inhib (%) | | |
| PIP5K1B FRET-Adapta SP | Test Conc(μM):ATP | 1:10 | Mean Mean | 2.1 | N |
| | Conc(μM) | | Inhib (%) | | |
| PIP5K1C FRET-Adapta SP | Test Conc(μM):ATP | 1:10 | Mean Mean | 3.2 | N |
| | Conc(μM) | | Inhib (%) | | |
| WNK1 FRET-Lantha SP | Test Conc(μM) | 1 | Mean Mean | 5.3 | N |
| | | | Inhib (%) | | |
| CDK4 Cyclin D1 *Mn* Adapta SP | Test Conc(μM):ATP | 1:10 | Mean Mean | 2.5 | N |
| | Conc(μM) | | Inhib (%) | | |
| CDK4 Cyclin D3 *Mn* Adapta SP | Test Conc(μM):ATP | 1:10 | Mean Mean | −3.9 | N |
| | Conc(μM) | | Inhib (%) | | |
| CDK13 Cyclin K FRET-Lantha SP | Test Conc(μM) | 1 | Mean Mean | 4.1 | N |
| | | | Inhib (%) | | |
| CDK11 Cyclin C FRET-Lantha SP | Test Conc(μM) | 1 | Mean Mean | 3.8 | N |
| | | | Inhib (%) | | |

### REFERENCES

Agabiti, S. S., Li, J., and Wiemer, A. J. (2017). Geranylgeranyl diphosphate synthase inhibition induces apoptosis that is dependent upon GGPP depletion, ERK phosphorylation and caspase activation. Cell death & disease 8, e2678.
Ashida, S., Kawada, C., and Inoue, K. (2017). Stromal regulation of prostate cancer cell growth by mevalonate pathway enzymes HMGCS1 and HMGCR. Oncology letters 14, 6533-6542.
Bassat, E., Mutlak, Y. E., Genzelinakh, A., Shadrin, I. Y., Baruch Umansky, K., Yifa, O., Kain, D., Rajchman, D., Leach, J., Riabov Bassat, D., et al. (2017). The extracellular matrix protein agrin promotes heart regeneration in mice. Nature 547, 179-184.
Brown, D. N., Caffa, I., Cirmena, G., Piras, D., Garuti, A., Gallo, M., Alberti, S., Nencioni, A., Ballestrero, A., and Zoppoli, G. (2016). Squalene epoxidase is a bona fide oncogene by amplification with clinical relevance in breast cancer. Scientific reports 6, 19435.
Bruderer, R., Bernhardt, O. M., Gandhi, T., Miladinovic, S. M., Cheng, L. Y., Messner, S., Ehrenberger, T., Zanotelli, V., Butscheid, Y., Escher, C., et al. (2015). Extending the limits of quantitative proteome profiling with data-independent acquisition and application to acetaminophen-treated three-dimensional liver microtissues. Molecular & cellular proteomics: MCP 14, 1400-1410.
Bruderer, R., Bernhardt, O. M., Gandhi, T., Xuan, Y., Sondermann, J., Schmidt, M., Gomez-Varela, D., and Reiter, L. (2017). Optimization of Experimental Parameters in Data-Independent Mass Spectrometry Significantly Increases Depth and Reproducibility of Results. Molecular & cellular proteomics: MCP 16, 2296-2309.
Clendening, J. W., Pandyra, A., Boutros, P. C., El Ghamrasni, S., Khosravi, F., Trentin, G. A., Martirosyan, A., Hakem, A., Hakem, R., Jurisica, I., et al. (2010). Dysregulation of the mevalonate pathway promotes transformation. Proceedings of the National Academy of Sciences of the United States of America 107, 15051-15056.
Cook, D., Brown, D., Alexander, R., March, R., Morgan, P., Satterthwaite, G., and Pangalos, M. N. (2014). Lessons learned from the fate of AstraZeneca's drug pipeline: a five-dimensional framework. Nat Rev Drug Discov 13, 419-431.
Cox, J., and Mann, M. (2008). MaxQuant enables high peptide identification rates, individualized p.p.b.-range mass accuracies and proteome-wide protein quantification. Nature biotechnology 26, 1367-1372.
D'Uva, G., Aharonov, A., Lauriola, M., Kain, D., Yahalom-Ronen, Y., Carvalho, S., Weisinger, K., Bassat, E., Rajchman, D., Yifa, O., et al. (2015). ERBB2 triggers mammalian heart regeneration by promoting cardiomyocyte dedifferentiation and proliferation. Nature cell biology 17, 627-638.
Engel, F. B., Hsieh, P. C., Lee, R. T., and Keating, M. T. (2006). FGF1/p38 MAP kinase inhibitor therapy induces cardiomyocyte mitosis, reduces scarring, and rescues function after myocardial infarction. Proceedings of the National Academy of Sciences of the United States of America 103, 15546-15551.
Engel, F. B., Schebesta, M., Duong, M. T., Lu, G., Ren, S., Madwed, J. B., Jiang, H., Wang, Y., and Keating, M. T. (2005). p38 MAP kinase inhibition enables proliferation of adult mammalian cardiomyocytes. Genes & development 19, 1175-1187.
Eulalio, A., Mano, M., Dal Ferro, M., Zentilin, L., Sinagra, G., Zacchigna, S., and Giacca, M. (2012). Functional screening identifies miRNAs inducing cardiac regeneration. Nature 492, 376-381.
Fan, F., He, Z., Kong, L. L., Chen, Q., Yuan, Q., Zhang, S., Ye, J., Liu, H., Sun, X., Geng, J., et al. (2016). Pharmacological targeting of kinases MST1 and MST2 augments tissue repair and regeneration. Science translational medicine 8, 352ra108.
Fazakerley, D. J., Chaudhuri, R., Yang, P., Maghzal, G. J., Thomas, K. C., Krycer, J. R., Humphrey, S. J., Parker, B. L., Fisher-Wellman, K. H., Meoli, C. C., et al. (2018). Mitochondrial CoQ deficiency is a common driver of mitochondrial oxidants and insulin resistance. eLife 7, e32111.
Fordyce, C. B., Roe, M. T., Ahmad, T. Libby, P., Borer, J. S., Hiatt, W. R., Bristow, M. R., Packer, M., Wasserman, S. M., Braunstein, N., et al. (2015). Cardiovascular drug development: is it dead or just hibernating? J Am Coll Cardiol 65, 1567-1582.
Gagnon-Bartsch, J. A., Jacob, L., and Speed, T. P. (2013). Removing unwanted variation from high dimensional data with negative controls. Berkeley: Tech Reports from Dep Stat Univ California, 1-112.
Giacomelli, E., Bellin, M., Sala, L., van Meer, B. J., Tertoolen, L. G., Orlova, V. V., and Mummery, C. L. (2017). Three-dimensional cardiac microtissues composed of cardiomyocytes and endothelial cells co-differentiated from human pluripotent stem cells. Development (Cambridge, England) 144, 1008-1017.
Goldberg, F. W., Ward, R. A., Powell, S. J., Debreczeni, J. E., Norman, R. A., Roberts, N. J., Dishington, A. P., Gingell, H. J., Wickson, K. F., and Roberts, A. L. (2009). Rapid generation of a high quality lead for transforming growth factor-beta (TGF-beta) type I receptor (ALK5). Journal of medicinal chemistry 52, 7901-7905.
Hargrove, T. Y., Friggeri, L., Wawrzak, Z., Sivakumaran, S., Yazlovitskaya, E. M., Hiebert, S. W., Guengerich, F. P., Waterman, M. R., and Lepesheva, G. I. (2016). Human sterol 14alpha-demethylase as a target for anticancer chemotherapy: towards structure-aided drug design. Journal of lipid research 57, 1552-1563.
Heallen, T., Zhang, M., Wang, J., Bonilla-Claudio, M., Klysik, E., Johnson, R. L., and Martin, J. F. (2011). Hippo pathway inhibits Wnt signaling to restrain cardiomyocyte proliferation and heart size. Science (New York, N.Y.) 332, 458-461.
Hekimian, A., Duan, L., Doctorian, T., and Lee, M.-S. (2018). Statin exposure during the first trimester of pregnancy is associated with fetal ventricular septal defects. Journal of the American College of Cardiology 71, A571.
Horvath, P., Aulner, N., Bickle, M., Davies, A. M., Nery, E. D., Ebner, D., Montoya, M. C., Ostling, P., Pietiainen, V., Price, L. S., et al. (2016). Screening out irrelevant cell-based models of disease. Nat Rev Drug Discov 15, 751-769.
Huang da, W., Sherman, B. T., and Lempicki, R. A. (2009). Systematic and integrative analysis of large gene lists using DAVID bioinformatics resources. Nature protocols 4, 44-57.
Hudson, J., Titmarsh, D., Hidalgo, A., Wolvetang, E., and Cooper-White, J. (2012). Primitive cardiac cells from human embryonic stem cells. Stem cells and development 21, 1513-1523.
Hudson, J. E., Brooke, G., Blair, C., Wolvetang, E., and Cooper-White, J. J. (2011). Development of myocardial constructs using modulus-matched acrylated polypropylene glycol triol substrate and different nonmyocyte cell populations. Tissue engineering Part A 17, 2279-2289.
Hughes, C. S., Foehr, S., Garfield, D. A., Furlong, E. E., Steinmetz, L. M., and Krijgsveld, J. (2014). Ultrasensitive proteome analysis using paramagnetic bead technology. Molecular systems biology 10, 757.
Jabs, J., Zickgraf, F. M., Park, J., Wagner, S., Jiang, X., Jechow, K., Kleinheinz, K., Toprak, U. H., Schneider, M. A., Meister, M., et al. (2017). Screening drug effects in patient-derived cancer cells links organoid responses to genome alterations. Molecular systems biology 13, 955.
Keber, R., Motaln, H., Wagner, K. D., Debeljak, N., Rassoulzadegan, M., Acimovic, J., Rozman, D., and Horvat, S. (2011). Mouse knockout of the cholesterogenic cytochrome P450 lanosterol 14alpha-demethylase (Cyp51) resembles Antley-Bixler syndrome. The Journal of biological chemistry 286, 29086-29097.
Kodo, K., Ong, S.-G., Jahanbani, F., Termglinchan, V., Hirono, K., InanlooRahatloo, K., Ebert, A. D., Shukla, P., Abilez, O. J., Churko, J. M., et al. (2016). iPSC-derived cardiomyocytes reveal abnormal TGF-β signalling in left ventricular non-compaction cardiomyopathy. Nature cell biology 18, 1031-1042.
Kuppusamy, K. T., Jones, D. C., Sperber, H., Madan, A., Fischer, K. A., Rodriguez, M. L., Pabon, L., Zhu, W. Z., Tulloch, N. L., Yang, X., et al. (2015). Let-7 family of microRNA is required for maturation and adult-like metabolism in stem cell-derived cardiomyocytes. Proc Natl Acad Sci USA 112, E2785-2794.
Leach, J. P., Heallen, T., Zhang, M., Rahmani, M., Morikawa, Y., Hill, M. C., Segura, A., Willerson, J. T., and Martin, J. F. (2017). Hippo pathway deficiency reverses systolic heart failure after infarction. Nature 550, 260-264.
Lin, Z., von Gise, A., Zhou, P., Gu, F., Ma, Q., Jiang, J., Yau, A. L., Buck, J. N., Gouin, K. A., van Gorp, P. R. R., et al. (2014). Cardiac-specific YAP activation improves cardiac function and survival in an experimental murine MI model. Circulation research 115, 354-363.
Love, M. I., Huber, W., and Anders, S. (2014). Moderated estimation of fold change and dispersion for RNA-seq data with DESeq2. Genome biology 15, 550.
Mannhardt, I., Breckwoldt, K., Letuffe-Brenière, D., Schaaf, S., Schulz, H., Neuber, C., Benzin, A., Werner, T., Eder, A., Schulze, T., et al. (2016). Human Engineered Heart Tissue: Analysis of Contractile Force. Stem Cell Reports 7, 29-42.
Matsa, E., Burridge, P. W., and Wu, J. C. (2014). Human stem cells for modeling heart disease and for drug discovery. Sci Transl Med 6, 239ps236.
Miettinen, T. P., and Bjorklund, M. (2015). Mevalonate Pathway Regulates Cell Size Homeostasis and Proteostasis through Autophagy. Cell reports 13, 2610-2620.
Mills, R. J., Titmarsh, D. M., Koenig, X., Parker, B. L., Ryall, J. G., Quaife-Ryan, G. A., Voges, H. K., Hodson, M. P., Ferguson, C., Drowley, L., et al. (2017a). Functional screening in human cardiac organoids reveals a metabolic mechanism for cardiomyocyte cell cycle arrest. Proceedings of the National Academy of Sciences of the United States of America 114, E8372-E8381.
Mills, R. J., Voges, H. K., Porrello, E. R., and Hudson, J. E. (2017b). Cryoinjury Model for Tissue Injury and Repair in Bioengineered Human Striated Muscle. Methods in molecular biology (Clifton, N.J.) 1668, 209-224.
Mills, R. J., Voges, H. K., Porrello, E. R., and Hudson, J. E. (2018). Disease modeling and functional screening using engineered heart tissue. Current Opinion in Physiology 1, 80-88.
Moffat, J. G., Vincent, F., Lee, J. A., Eder, J., and Prunotto, M. (2017). Opportunities and challenges in phenotypic drug discovery: an industry perspective. Nat Rev Drug Discov 16, 531-543.
Mohamed, T. M. A., Ang, Y. S., Radzinsky, E., Zhou, P., Huang, Y., Elfenbein, A., Foley, A., Magnitsky, S., and Srivastava, D. (2018). Regulation of Cell Cycle to Stimulate Adult Cardiomyocyte Proliferation and Cardiac Regeneration. Cell 173, 104-116.e112.
Morikawa, Y., Zhang, M., Heallen, T., Leach, J., Tao, G., Xiao, Y., Bai, Y., Li, W., Willerson, J. T., and Martin, J. F. (2015). Actin cytoskeletal remodeling with protrusion formation is essential for heart regeneration in Hippo-deficient mice. Science signaling 8, ra41.
Nakada, Y Canseco, D. C., Thet, S., Abdisalaam, S., Asaithamby, A., Santos, C. X Shah, A. M., Zhang, H., Faber, J. E., Kinter, M. T., et al. (2017). Hypoxia induces heart regeneration in adult mice. Nature 541, 222-227.
Pool, F., Currie, R., Sweby, P. K., Salazar, J. D., and Tindall, M. J. (2018). A mathematical model of the mevalonate cholesterol biosynthesis pathway. Journal of theoretical biology 443, 157-176.
Porrello, E. R., Mahmoud, A. I., Simpson, E., Hill, J. A., Richardson, J. A., Olson, E. N., and Sadek, H. A. (2011). Transient regenerative potential of the neonatal mouse heart. Science (New York, N.Y.) 331, 1078-1080.
Porrello, E. R., Mahmoud, A. I., Simpson, E., Johnson, B. A., Grinsfelder, D., Canseco, D., Mammen, P. P., Rothermel, B. A., Olson, E. N., and Sadek, H. A. (2013). Regulation of neonatal and adult mammalian heart regeneration by the miR-15 family. Proceedings of the National Academy of Sciences of the United States of America 110, 187-192.
Quaife-Ryan, G. A., Sim, C. B., Ziemann, M., Kaspi, A., Rafehi, H., Ramialison, M., El-Osta, A., Hudson, J. E., and Porrello, E. R. (2017). Multicellular Transcriptional Analysis of Mammalian Heart Regeneration. Circulation 136, 1123-1139.
Rosenbluh, J., Nijhawan, D., Cox, A. G., Li, X., Neal, J. T., Schafer, E. J., Zack, T. I., Wang, X., Tsherniak, A., Schinzel, A. C., et al. (2012). beta-Catenin-driven cancers require a YAP1 transcriptional complex for survival and tumorigenesis. Cell 151, 1457-1473.
Salami, J. A., Warraich, H., Valero-Elizondo, J., Spatz, E. S., Desai, N. R., Rana, J. S., Virani, S. S., Blankstein, R., Khera, A., Blaha, M. J., et al. (2017). National Trends in Statin Use and Expenditures in the US Adult Population From 2002 to 2013: Insights From the Medical Expenditure Panel Survey. JAMA cardiology 2, 56-65.
Shadrin, I. Y., Allen, B. W., Qian, Y., Jackman, C. P., Carlson, A. L., Juhas, M. E., and Bursac, N. (2017). Cardiopatch platform enables maturation and scale-up of human pluripotent stem cell-derived engineered heart tissues. Nature communications 8, 1825.
Sorrentino, G., Ruggeri, N., Specchia, V., Cordenonsi, M., Mano, M., Dupont, S., Manfrin, A., Ingallina, E., Sommaggio, R., Piazza, S., et al. (2014). Metabolic control of YAP and TAZ by the mevalonate pathway. Nature cell biology 16, 357-366.
Stoekenbroek, R. M., Hartgers, M. L., Rutte, R., de Wijer, D. D., Stroes, E. S. G., and Hovingh, G. K. (2018). PCSK9 inhibitors in clinical practice: Delivering on the promise? Atherosclerosis 270, 205-210.
Sui, Z., Zhou, J., Cheng, Z., and Lu, P. (2015). Squalene epoxidase (SQLE) promotes the growth and migration of the hepatocellular carcinoma cells. Tumour biology: the journal of the International Society for Oncodevelopmental Biology and Medicine 36, 6173-6179.
Tao, G., Kahr, P. C., Morikawa, Y., Zhang, M., Rahmani, M., Heallen, T. R., Li, L., Sun, Z., Olson, E. N., Amendt, B. A., et al. (2016). Pitx2 promotes heart repair by activating the antioxidant response after cardiac injury. Nature 534, 119-123.
Team, R. C. (2015). R: A language and environment for statistical computing. R Foundation for Statistical Computing Vienna, Austria, http://www.R-project.org/.
Tiburcy, M., Hudson, J. E., Balfanz, P., Schlick, S., Meyer, T., Chang Liao, M. L., Levent, E., Raad, F., Zeidler, S., Wingender, E., et al. (2017). Defined Engineered Human Myocardium With Advanced Maturation for Applications in Heart Failure Modeling and Repair. Circulation 135, 1832-1847.
Titmarsh, D. M., Glass, N. R., Mills, R. J Hidalgo, A., Wolvetang, E. J., Porrello, E. R., Hudson, J. E., and Cooper-White, J. J. (2016). Induction of Human iPSC-Derived Cardiomyocyte Proliferation Revealed by Combinatorial Screening in High Density Microbioreactor Arrays. Scientific reports 6, 24637.
Ulmer, B. M., Stoehr, A., Schulze, M. L., Patel, S., Gucek, M., Mannhardt, I., Funcke, S., Murphy, E., Eschenhagen, T., and Hansen, A. (2018). Contractile Work Contributes to Maturation of Energy Metabolism in hiPSC-Derived Cardiomyocytes. Stem Cell Reports 10, 834-847.
Vander Heiden, M. G., Cantley, L. C., and Thompson, C. B. (2009). Understanding the Warburg Effect: The Metabolic Requirements of Cell Proliferation. Science (New York, N.Y.) 324, 1029-1033.
Vlachogiannis, G., Hedayat, S., Vatsiou, A., Jamin, Y., Fernandez-Mateos, J., Khan, K., Lampis, A., Eason, K., Huntingford, I., Burke, R., et al. (2018). Patient-derived organoids model treatment response of metastatic gastrointestinal cancers. Science (New York, N.Y.) 359, 920-926.
Voges, H. K., Mills, R. J., Elliott, D. A., Parton, R. G., Porrello, E. R., and Hudson, J. E. (2017). Development of a human cardiac organoid injury model reveals innate regenerative potential. Development (Cambridge, England) 144, 1118-1127.
Yang, X., Pabon, L., and Murry, C. E. (2014). Engineering adolescence: maturation of human pluripotent stem cell-derived cardiomyocytes. Circulation research 114, 511-523.
Ye, L., D'Agostino, G., Loo, Si., Wang, C. X., Su, L. P., Tan, S. H., Tee, G. Z., Pua, C. J., Pena, E. M., Cheng, R. B., et al. (2018). Early Regenerative Capacity in the Porcine Heart. Circulation.
Young, M. D., Wakefield, M. J., Smyth, G. K., and Oshlack, A. (2010). Gene ontology analysis for RNA-seq: accounting for selection bias. Genome biology 11, R14.
Yu, G., Li, F., Qin, Y. Bo, X., Wu, Y., and Wang, S. (2010). GOSemSim: an R package for measuring semantic similarity among GO terms and gene products. Bioinformatics (Oxford, England) 26, 976-978.
Zanconato, F., Forcato, M., Battilana, G., Azzolin, L., Quaranta, E., Bodega, B., Rosato, A., Bicciato, S., Cordenonsi, M., and Piccolo, S. (2015). Genome-wide association between YAP/TAZ/TEAD and AP-1 at enhancers drives oncogenic growth. Nature cell biology 17, 1218-1227.
Zhou, J., Ahmad, F., Parikh, S., Hoffman, N. E., Rajan, S., Verma, V. K., Song, J., Yuan, A., Shanmughapriya, S., Guo, Y., et al. (2016). Loss of Adult Cardiac Myocyte GSK-3 Leads to Mitotic Catastrophe Resulting in Fatal Dilated Cardiomyopathy. Circulation research 118, 1208-1222.
Zhu, W., Zhang, E., Zhao, M., Chong, Z., Fan, C., Tang, Y., Hunter, J. D., Borovjagin, A. V., Walcott, G. P Chen, J. Y., et al. (2018). Regenerative Potential of Neonatal Porcine Hearts. Circulation.
Al Adhami, H., Evano, B., Le Digarcher, A., Gueydan, C., Dubois, E., Parrinello, H., Dantec, C., Bouschet, T., Varrault, A., and Journot, L. (2015). A systems-level approach to parental genomic imprinting: the imprinted gene network includes extracellular matrix genes and regulates cell cycle exit and differentiation. Genome research 25, 353-367.
Charron, G., Li, M. M., MacDonald, M. R., and Hang, H. C. (2013). Prenylome profiling reveals S-farnesylation is crucial for membrane targeting and antiviral activity of ZAP long-isoform. Proceedings of the National Academy of Sciences of the United States of America 110, 11085-11090.
Gillet, L. C., Navarro, P., Tate, S., Rost, H., Selevsek, N., Reiter, L., Bonner, R., and Aebersold, R. (2012). Targeted data extraction of the MS/MS spectra generated by data-independent acquisition: a new concept for consistent and accurate proteome analysis. Molecular & cellular proteomics: MCP 11, O111 016717.
Kho, Y., Kim, S. C., Jiang, C., Barma, D., Kwon, S. W., Cheng, J., Jaunbergs, J., Weinbaum, C., Tamanoi, F., Falck, J., et al. (2004). A tagging-via-substrate technology for detection and proteomics of farnesylated proteins. Proceedings of the National Academy of Sciences of the United States of America 101, 12479-12484.
Qiao, H., Li, Y., Feng, C., Duo, S., Ji, F., and Jiao, J. (2018). Nap111 Controls Embryonic Neural Progenitor Cell Proliferation and Differentiation in the Developing Brain. Cell reports 22, 2279-2293.
Rodriguez, P., Pelletier, J., Price, G. B., and Zannis-Hadjopoulos, M. (2000). NAP-2: histone chaperone function and phosphorylation state through the cell cycle. Journal of molecular biology 298, 225-238.
Schimmack, S., Taylor, A., Lawrence, B., Alaimo, D., Schmitz-Winnenthal, H., Buehler, M. W., Modlin, I. M., and Kidd, M. (2014). A mechanistic role for the chromatin modulator, NAP1L1, in pancreatic neuroendocrine neoplasm proliferation and metastases. Epigenetics & Chromatin 7, 15-15.
Yan, Y., Yin, P., Gong, H., Xue, Y., Zhang, G., Fang, B., Chen, Z., Li, Y., Yang, C., Huang, Z., et al. (2016). Nucleosome Assembly Protein 1-Like 1 (Nap111) Regulates the Proliferation of Murine Induced Pluripotent Stem Cells. Cellular physiology and biochemistry: international journal of experimental cellular physiology, biochemistry, and pharmacology 38, 340-350.
MacLean, B., Tomazela, D. M., Shulman, N., Chambers, M., Finney, G. L., Frewen, B., Kern, R., Tabb, D. L., Liebler, D. C., and MacCoss, M. J. (2010). Skyline: an open source document editor for creating and analyzing targeted proteomics experiments. Bioinformatics (Oxford, England) 26, 966-968.

1. A method of inducing cardiomyocyte proliferation in vitro, the method including the step of contacting one or a plurality of cardiomyocytes with an effective amount of an agent capable of at least partly activating sterol biosynthesis therein to thereby induce cardiomyocyte proliferation.

2. A method of inducing cardiomyocyte proliferation in a subject, the method including the step of administering to the subject an effective amount of an agent capable of at least partly activating sterol biosynthesis in a cardiomyocyte to thereby induce cardiomyocyte proliferation in the subject.

3. A method of regenerating a cardiac tissue in a subject in need thereof, the method including the step of administering to the subject a therapeutically effective amount of an agent capable of at least partly activating sterol biosynthesis in a cardiomyocyte to thereby treat or repair the cardiac damage in the subject.

4. The method of claim 3, wherein the agent is capable of promoting or inducing cardiomyocyte proliferation in the subject.

5. The method of claim 3 or claim 4, wherein the subject has or is at risk of developing a cardiac disease, disorder or condition selected from the group consisting of a myocardial infarction, a congestive heart failure, tachyarrhythmia, familial hypertrophic cardiomyopathy, ischemic heart disease, idiopathic dilated cardiomyopathy, congenital heart disease and myocarditis.

6. The method of any one of claims 2 to 5, wherein administering the agent comprises oral administration, intravenous injection, topical administration, myocardial injection, an implantable device and any combination thereof.

7. The method of any one of the preceding claims, wherein the agent is or comprises a p38α inhibitor, a MST1 inhibitor, a TGF-beta receptor inhibitor and/or a BMP receptor inhibitor.

8. The method of any one of the preceding claims, wherein activating sterol biosynthesis comprises, at least in part, increasing the expression and/or activity of one or more proteins and/or enzymes of, or associated with, sterol biosynthesis.

9. The method of claim 8, wherein the one or more proteins and/or enzymes are selected from the group consisting of squalene monooxygenase (SQLE), Hydroxymethylglutaryl(HMG)-CoA synthase (HMGCS1), Lanosterol 14 alpha-demethylase (CYP51A1), HMG-CoA reductase (HMGCR), Hydroxymethylglutaryl(HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase, a farnesyltransferase, sterol regulatory element binding protein 1 (SREBP1), sterol regulatory element binding protein 2 (SREBP2) and any combination thereof.

10. The method of any one of the preceding claims, wherein the agent is further capable of at least partly modulating the expression and/or activity of a cell cycle protein.

11. The method of claim 10, wherein the cell cycle protein is selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

12. The method or composition of any one of the preceding claims, wherein the agent maintains, at least in part, contractile function of proliferated cardiomyocytes.

13. A composition for use in regenerating a cardiac tissue in a subject, the composition comprising a therapeutically effective amount of an agent capable of activating sterol biosynthesis and optionally a pharmaceutically acceptable carrier, diluent or excipient.

14. The composition of claim 13, for use in the method of any one of claims 1 to 12.

15. A method of screening, designing, engineering or otherwise producing an agent for inducing cardiomyocyte proliferation, said method including steps of:
(a) contacting one or a plurality of cardiomyocytes with a candidate molecule; and
(b) determining whether the candidate molecule is capable of at least partly activating sterol biosynthesis to thereby induce cardiomyocyte proliferation.

16. The method of claim 15, wherein step (b) comprises determining whether the candidate molecule activates and/or increases the expression of one or more proteins and/or enzymes of, or associated with, sterol biosynthesis.

17. The method of claim 16, wherein the one or more proteins and/or enzymes are selected from the group of squalene monooxygenase (SQLE), Hydroxymethylglutaryl(HMG)-CoA synthase (HMGCS1), Lanosterol 14 alpha-demethylase (CYP51A1), HMG-CoA reductase (HMGCR), Hydroxymethylglutaryl(HMG)-CoA synthase 2 (mitochondrial; HMGCS2), Isopentenyl pyrophosphate isomerase (IPP isomerase; IDI1), pyrophosphomevalonate decarboxylase (MVD), 24-Dehydrocholesterol reductase (DHCR24), NAD(P)H steroid dehydrogenase-like protein (NSDHL), farnesyl diphosphate synthase (FDPS), farnesyl-diphosphate farnesyltransferase 1 (FDFT1), methylsterol monooxygenase 1 (MSMO1), Mevalonate kinase (MVK), a geranylgeranyltransferase, a farnesyltransferase, Sterol regulatory element-binding protein 1 (SREBP1), Sterol regulatory element-binding protein 2 (SREBP2) and any combination thereof.

18. The method of any one of claims 15 to 17, including the further step of determining whether the candidate molecule is capable of at least partly modulating the expression and/or activity of a cell cycle protein.

19. The method of claim 18, wherein the cell cycle protein is selected from the group consisting of polo-like kinase 1 (PLK-1), Cyclin B2 (CCNB2), Cyclin D1 (CCND1), Cyclin A2 (CCNA2), Forkhead box protein M1 (FOXM1), Cyclin-dependent kinase 4 inhibitor B (CDKN2B), Aurora B kinase (AURKB) and any combination thereof.

20. The method of any one of claims 15 to 19, wherein the one or plurality of cardiomyocytes are or comprise a cardiac organoid.

21. An agent for inducing cardiomyocyte proliferation screened, designed, engineered or otherwise produced according to the method of any one of claims 15 to 20.

22. The agent for inducing cardiomyocyte proliferation of claim 21, for use according to the method of any one of claims 1 to 12.

23. The method of any one of claims 1 to 12 and 15 to 20, the composition of claim 13 or claim 14 or the agent of claim 21 or 22, wherein activating sterol biosynthesis comprises activating mevalonate biosynthesis and/or isoprenoid biosynthesis.
